(19) United States
(12) Patent Application Publication
(10) Pub. No.: US 2024/0165097 A1
(43) Pub. Date: May 23, 2024

(54) PHARMACEUTICAL COMPOSITION FOR THE TREATMENT OF ALZHEIMER'S DISEASE OR DEMENTIA

(71) Applicant: Heptares Therapeutics Limited, Cambridge (GB)

(72) Inventor: Tim Tasker, Cambridge (GB)

(21) Appl. No.: 18/280,611

(22) PCT Filed: Mar. 7, 2022
(86) PCT No.: PCT/EP2022/055775

§ 371 Date: Sep. 6, 2023

(30) Foreign Application Priority Data
Mar. 8, 2021 (GB) 2103211.5

(51) Int. Cl. A61K 31/46; A61K 31/445; A61P 25/28

(52) U.S. Cl. CPC A61K 31/46; A61K 31/445; A61P 25/28

(57) ABSTRACT

This invention relates to the use of compounds which are agonists of the muscarinic M₁ receptor and M₄ receptor, their salts and pharmaceutical compositions containing them in therapy of the human body. In particular, the invention is directed to compounds, which are useful in the treatment of diseases mediated by the muscarinic M₁/M₄ receptors, including neurodegenerative disorders (i.e. Alzheimer's disease) and neuropsychiatric disorders (Schizophrenia).

This invention relates to the use of compounds, their salts, pharmaceutical compositions containing them in therapy of the human body. In particular, the invention is directed to compounds, which are agonists of the muscarinic M₁ receptor and M₄ receptor, and hence are useful in the treatment of diseases mediated by the muscarinic M₁/M₄ receptors, including neurodegenerative disorders (i.e. Alzheimer's disease) and neuropsychiatric disorders (Schizophrenia).

### BACKGROUND OF THE INVENTION

Muscarinic acetylcholine receptors (mAChRs) are members of the G protein-coupled receptor superfamily which mediate the actions of the neurotransmitter acetylcholine in both the central and peripheral nervous system. Five mAChR subtypes have been cloned, M₁ to M₅. The M₁ mAChR is predominantly expressed post-synaptically in the cortex, hippocampus, striatum and thalamus; M₂ mAChRs are located predominantly in the brainstem and thalamus, though also in the cortex, hippocampus and striatum where they reside on cholinergic synaptic terminals (Langmead et al., 2008 Br J Pharmacol). However, M₂ mAChRs are also expressed peripherally on cardiac tissue (where they mediate the vagal innervation of the heart) and in smooth muscle and exocrine glands. M₃ mAChRs are expressed at relatively low level in the CNS but are widely expressed in smooth muscle and glandular tissues such as sweat and salivary glands (Langmead et al., 2008 Br J Pharmacol).

Muscarinic receptors in the central nervous system, especially the M₁ mAChR, play a critical role in mediating higher cognitive processing. Diseases associated with cognitive impairments, such as Alzheimer's disease, are accompanied by loss of cholinergic neurons in the basal forebrain (Whitehouse et al., 1982 Science). In schizophrenia, which is also characterised by cognitive impairments, mAChR expression is reduced in the dorsolateral pre-frontal cortex, hippocampus and caudate putamen in post-mortem tissues of patients diagnosed with schizophrenia (Dean et al., 2002 Mol Psychiatry). Furthermore, in animal models, blockade or lesion of central cholinergic pathways results in profound cognitive deficits. Non-selective mAChR antagonists have also been shown to induce cognitive deficits and psychotomimetic effects in healthy volunteers, and aggravate behavioural and cognitive symptoms in patients diagnosed with a psychotic disorder. Cholinergic replacement therapy has largely been based on the use of acetylcholinesterase inhibitors to prevent the breakdown of endogenous acetylcholine. These compounds have shown efficacy on symptomatic cognitive decline in the clinic, but give rise to dose-limiting side effects resulting from stimulation of peripheral M₂ and M₃ mAChRs including disturbed gastrointestinal motility, bradycardia, nausea and vomiting (http://www.drugs.com/pro/donepezil.html; http://www.drugs.com/pro/rivastidmine.html). Further discovery efforts have targeted the identification of direct M₁ mAChR agonists to improve cognitive function. Such efforts resulted in the identification of a range of agonists, exemplified by compounds such as xanomeline, AF267B, sabcomeline, milameline and cevimeline. Many of these compounds have been shown to be highly effective in pre-clinical models of cognition in both rodents and/or non-human primates. Milameline has shown efficacy recovering scopolamine-induced deficits in working and spatial memory in rodents; sabcomeline displayed efficacy in a visual object discrimination task in marmosets and xanomeline reversed mAChR antagonist-induced deficits in cognitive performance in a passive avoidance paradigm.

Alzheimer's disease (AD) is the most common neurodegenerative disorder (26.6 million people worldwide in 2006) that affects the elderly, resulting in profound cognitive dysfunction, with executive functioning, and learning and memory processes being most affected. The aetiology of the disease is complex but is characterised by two hallmark brain sequelae: aggregates of amyloid plaques, largely composed of amyloid-p peptide (Aβ), and neurofibrillary tangles, formed by hyperphosphorylated tau proteins. The accumulation of Aβ is thought to be the central feature in the progression of AD and, as such, many putative therapies for the treatment of AD are currently targeting inhibition of Aβ production. Aβ is derived from proteolytic cleavage of the membrane bound amyloid precursor protein (APP). APP is processed by two routes, non-amyloidgenic and amyloidgenic. Cleavage of APP by γ-secretase is common to both pathways, but in the former APP is cleaved by an α-secretase to yield soluble APPα. The cleavage site is within the Aβ sequence, thereby precluding its formation. However, in the amyloidgenic route, APP is cleaved by β-secretase to yield soluble APPβ and also Aβ. In vitro studies have shown that mAChR agonists can promote the processing of APP toward the soluble, non-amyloidogenic pathway. In vivo studies showed that the mAChR agonist, AF267B, altered disease-like pathology in the 3xTgAD transgenic mouse, a model of the different components of Alzheimer's disease (Caccamo et al., 2006 Neuron). Finally, the mAChR agonist cevimeline has been shown to give a small, but significant, reduction in cerebrospinal fluid levels of Aβ in Alzheimer's patients, thus demonstrating potential disease modifying efficacy (Nitsch et al., 2000 Neurol).

35 Furthermore, preclinical studies have suggested that mAChR agonists display an atypical antipsychotic-like profile in a range of pre-clinical paradigms. The mAChR agonist, xanomeline, reverses a number of dopamine driven behaviours, including amphetamine induced locomotion in rats, apomorphine induced climbing in mice, dopamine agonist driven turning in unilateral 6-OH-DA lesioned rats and amphetamine induced motor unrest in monkeys (without EPS liability). It also has been shown to inhibit A10, but not A9, dopamine cell firing and conditioned avoidance and induces c-fos expression in prefrontal cortex and nucleus accumbens, but not in other striatal areas in the rat. These data are all suggestive of an atypical antipsychotic-like profile (Mirza et al., 1999 CNS Drug Rev). Muscarinic receptors have also been implicated in the neurobiology of addiction. The reinforcing effects of cocaine and other addictive substances are mediated by the mesolimbic dopamine system where behavioural and neurochemical studies have shown that the cholinergic muscarinic receptor subtypes play important roles in regulation of dopaminergic neurotransmission. For example M(4) (−/−) mice demonstrated significantly enhanced reward driven behaviour as result of exposure to cocaine (Schmidt et al Psychopharmacology (2011) August; 216(3):367-78). Furthermore, xanomeline has been demonstrated to block the effects of cocaine in these models.

Muscarinic receptors are also involved in the control of movement and potentially represent novel treatments for movement disorders such as Parkinson's disease, ADHD, Huntingdon's disease, Tourette's syndrome and other syndromes associated with dopaminergic dysfunction as an underlying pathogenetic factor driving disease.

Xanomeline, sabcomeline, milameline and cevimeline have all progressed into various stages of clinical development for the treatment of Alzheimer's disease and/or schizophrenia. Phase II clinical studies with xanomeline demonstrated its efficacy on cognitive symptom domains, and additionally showed improvements on behavioural disturbances, including delusional ideation, agitation and hallucinations associated with Alzheimer's disease (Bodick et al., 1997 Arch Neurol). This compound was also assessed in a small Phase II study in patients with chronic Schizophrenia and gave a significant reduction in positive and negative symptoms when compared to placebo control, and improved scores on episodic memory (Shekhar et al., 2008 Am J Psych). However, in all clinical studies xanomeline and other related mAChR agonists have displayed an unacceptable safety margin with respect to cholinergic side effects, including nausea, gastrointestinal pain, diarrhoea, diaphoresis (excessive sweating), hypersalivation (excessive salivation), syncope and bradycardia.

Muscarinic receptors are involved in central and peripheral pain. Pain can be divided into three different types: acute, inflammatory, and neuropathic. Acute pain serves an important protective function in keeping the organism safe from stimuli that may produce tissue damage however management of post-surgical pain is required. Inflammatory pain may occur for many reasons including tissue damage, autoimmune response, and pathogen invasion and is triggered by the action of inflammatory mediators such as neuropeptides and prostaglandins which result in neuronal inflammation and pain. Neuropathic pain is associated with abnormal painful sensations to non-painful stimuli. Neuropathic pain is associated with a number of different diseases/traumas such as spinal cord injury, multiple sclerosis, diabetes (diabetic neuropathy), viral infection (such as HIV or Herpes). It is also common in cancer both as a result of the disease or a side effect of chemotherapy. Activation of muscarinic receptors has been shown to be analgesic across a number of pain states through the activation of receptors in the spinal cord and higher pain centres in the brain. Increasing endogenous levels of acetylcholine through acetylcholinesterase inhibitors, direct activation of muscarinic receptors with agonists or allosteric modulators has been shown to have analgesic activity. In contrast blockade of muscarinic receptors with antagonists or using knockout mice increases pain sensitivity. Evidence for the role of the M₁ receptor in pain is reviewed by D. F. Fiorino and M. Garcia-Guzman, 2012.

More recently, a small number of compounds have been identified which display improved selectivity for the M₁ mAChR subtype over the peripherally expressed mAChR subtypes (Bridges et al., 2008 Bioorg Med Chem Lett; Johnson et al., 2010 Bioorg Med Chem Lett; Budzik et al., 2010 ACS Med Chem Lett). Despite increased levels of selectivity versus the M₃ mAChR subtype, some of these compounds retain significant agonist activity at both this subtype and the M₂ mAChR subtype. Herein we describe a series of compounds which unexpectedly display high levels of selectivity for the M₁ and/or M₄ mAChR over the M₂ and M₃ receptor subtypes.

WO2015/140559 discloses spirocyclic compounds as muscarinic receptor agonists.

### THE INVENTION

The present invention provides a pharmaceutical composition comprising the compound ethyl 3-(3-oxo-2,8-diazaspiro[4.5]dec-8-yl)-8-azabicyclo[3.2.1]octane-8-carboxylate, or a pharmaceutically acceptable salt thereof, for use in the treatment of Alzheimer's Disease or dementia;

wherein the load dose of the compound or pharmaceutically acceptable salt thereof is between 5 to 25 mg.

The compound ethyl 3-(3-oxo-2,8-diazaspiro[4.5]dec-8-yl)-8-azabicyclo[3.2.1]octane-8-carboxylate is:

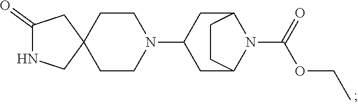 [SMILES: CCOC(=O)N1C2CCC1CC(N1CCC3(CC1)CNC(=O)C3)C2]

or a pharmaceutically acceptable salt thereof.

The compound may be:

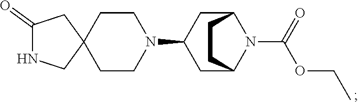

or a pharmaceutically acceptable salt thereof.

The compound may be:

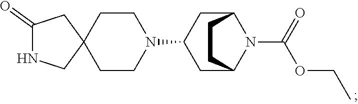

or a pharmaceutically acceptable salt thereof.

The compound may be ethyl (1R,3s,5S)-3-(3-oxo-2,8-diazaspiro[4.5]decan-8-yl)-8-azabicyclo[3.2.1]octane-8-carboxylate, or a pharmaceutically acceptable salt thereof.

The compound may be ethyl (1R,3r,5S)-3-(3-oxo-2,8-diazaspiro[4.5]decan-8-yl)-8-azabicyclo[3.2.1]octane-8-carboxylate, or a pharmaceutically acceptable salt thereof.

The composition of the invention may comprise any ratio of the diastereomers described above. For example the composition may comprise a racemic mixture of ethyl 3-(3-oxo-2,8-diazaspiro[4.5]dec-8-yl)-8-azabicyclo[3.2.1]octane-8-carboxylate. Alternatively, ethyl (1R,3s,5S)-3-(3-oxo-2,8-diazaspiro[4.5]clecan-8-yl)-8-azabicyclo[3.2.1]octane-8-carboxylate may be present in greater quantities than ethyl (1R,3r,5S)-3-(3-oxo-2,8-diazaspiro[4.5]decan-8-yl)-8-azabicyclo[3.2.1]octane-8-carboxylate. Likewise ethyl (1 R,3r,5S)-3-(3-oxo-2,8-diazaspiro[4.5]clecan-8-yl)-8-azabicyclo[3.2.1]octane-8-carboxylate may be present in greater quantities than ethyl (1R,3s,5S)-3-(3-oxo-2,8-diazaspiro[4.5]decan-8-yl)-8-azabicyclo[3.2.1]octane-8-carboxylate. Alternatively the composition may comprise exclusively ethyl (1 R,3s,5S)-3-(3-oxo-2,8-diazaspiro[4.5]clecan-8-yl)-8-azabicyclo[3.2.1]octane-8-carboxylate, or a pharmaceutically acceptable salt thereof. The composition may comprise exclusively ethyl (1R,3r,5S)-3-(3-oxo-2,8-diazaspiro[4.5]decan-8-yl)-8-azabicyclo[3.2.1]octane-8-carboxylate, or a pharmaceutically acceptable salt thereof.

The compound may be a pharmaceutically acceptable salt of ethyl 3-(3-oxo-2,8-diazaspiro[4.5]dec-8-yl)-8-azabicyclo[3.2.1]octane-8-carboxylate. The compound may be a pharmaceutically acceptable salt of ethyl (1R,3s,5S)-3-(3-oxo-2,8-diazaspiro[4.5]decan-8-yl)-8-azabicyclo[3.2.1]octane-8-carboxylate. The compound may be a pharmaceutically acceptable salt of ethyl (1R,3r,5S)-3-(3-oxo-2,8-diazaspiro[4.5]decan-8-yl)-8-azabicyclo[3.2.1]octane-8-carboxylate.

The compound may be:

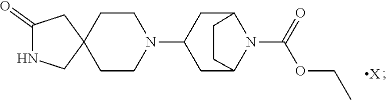

wherein X represents a pharmaceutically acceptable salt.

The compound may be:

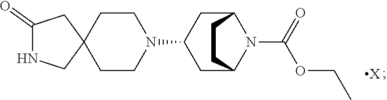

wherein X represents a pharmaceutically acceptable salt.

The compound may be:

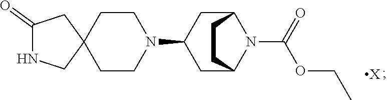

wherein X represents a pharmaceutically acceptable salt.

X represents a pharmaceutically acceptable salt. X may represent an acid addition salt. X may represent a hydrochloride salt. X may represent a monohydrochloride salt. X can be hydrochloride. X can be monohydrochloride. X can be HCl. X may represent a hydrobromide salt. X may represent a monohydrobromide salt. X may represent a maleate salt. X may represent a dihydrogenphosphate salt. X may represent a succinate salt. X may represent a tartrate salt. X can be hydrobromide. X can be monohydrobromide. H can be HBr. X can be maleate. X can be dihydrogenphosphate. X can be H₃PO₄. X can be succinate. X can be tartrate.

The compound may be:

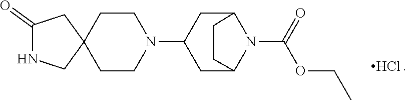 [SMILES: CCOC(=O)N1C2CCC1CC(N1CCC3(CC1)CNC(=O)C3)C2 ; Cl]

The compound may be:

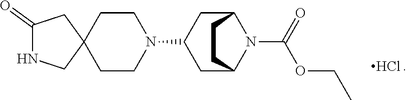

The compound may be:

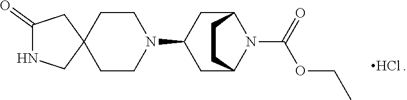

In the composition for use of the present invention the load dose of the compound ethyl 3-(3-oxo-2,8-diazaspiro[4.5]dec-8-yl)-8-azabicyclo[3.2.1]octane-8-carboxylate or pharmaceutically acceptable salt thereof is between 5 to 25 mg.

The load dose of the compound may be 5 to 20 mg. The load dose of the compound may be 5 to 15 mg. The load dose of the compound may be 5 to 10 mg. The load dose of the compound may be 10 to 25 mg. The load dose of the compound may be 10 to 20 mg. The load dose of the compound may be 10 to 15 mg. The load dose of the compound may be 15 to 25 mg. The load dose of the compound may be 15 to 20 mg. The load dose of the compound may be 20 to 25 mg. The load dose of the compound may be 5 mg. The load dose of the compound may be 10 mg. The load dose of the compound may be 15 mg. The load dose of the compound may be 20 mg. The load dose of the compound may be 25 mg.

In the composition for use of the present invention the compound ethyl 3-(3-oxo-2,8-diazaspiro[4.5]dec-8-yl)-8-azabicyclo[3.2.1]octane-8-carboxylate may be co-administered with a standard of care cholinesterase inhibitor (ChEI). In particular, the standard of care cholinesterase inhibitor may be Donepezil (2-[(1-benzylpiperidin-4-yl)methyl]-5,6-dimethoxy-2,3-dihydro-1 H-inden-1-one):

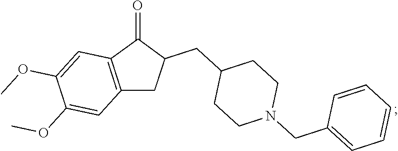 [SMILES: COc1cc2c(cc1OC)C(=O)C(CC1CCN(Cc3ccccc3)CC1)C2]

or a pharmaceutically acceptable salt thereof.

In the composition for use of the present invention the compound ethyl 3-(3-oxo-2,8-diazaspiro[4.5]dec-8-yl)-8-azabicyclo[3.2.1]octane-8-carboxylate may be co-administered with Donepezil. The compound ethyl 3-(3-oxo-2,8-diazaspiro[4.5]dec-8-yl)-8-azabicyclo[3.2.1]octane-8-carboxylate may be co-administered with Donepezil at a load dose of 5 to 25 mg. The compound ethyl 3-(3-oxo-2,8-diazaspiro[4.5]dec-8-yl)-8-azabicyclo[3.2.1]octane-8-carboxylate may be co-administered with Donepezil at a load dose of 10 mg.

Other cholinesterase inhibitors (ChEIs) that may be suitable for co-adminstration include but are not limited to:

Rivastigmine (3-[(1S)-1-(dimethylamino)ethyl]phenyl ethyyl(methyl)carbamate):

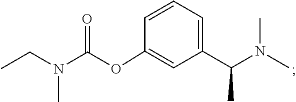 [SMILES: CCN(C)C(=O)Oc1cccc([C@H](C)N(C)C)c1]

Galantamine ((4aS)-3α-galanthamine):

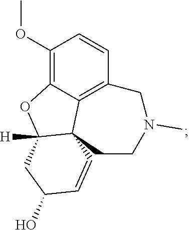 [SMILES: COc1ccc2c3c1O[C@H]1C[C@@H](O)C=C[C@@]31CCN(C)C2]

Tacrine (1,2,3,4-tetrahydroacridin-9-amine):

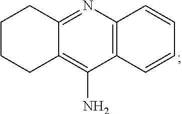 [SMILES: Nc1c2c(nc3ccccc13)CCCC2]

and pharmaceutically acceptable salts thereof.
the composition for use of the present invention the compound ethyl 3-(3-oxo-2,8-diazaspiro[4.5]dec-8-yl)-8-azabicyclo[3.2.1]octane-8-carboxylate may also be co-administered with other therapeutic agents useful in the treatment of dementia or Alzheimer's disease, including memantine:

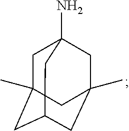

and pharmaceutically acceptable salts thereof.

### Definitions

In this application, the following definitions apply, unless indicated otherwise.

The term “treatment”, in relation to the uses of the compounds described herein is used to describe any form of intervention where a compound is administered to a subject suffering from, or at risk of suffering from, or potentially at risk of suffering from the disease or disorder in question. Thus, the term “treatment” covers both preventative (prophylactic) treatment and treatment where measurable or detectable symptoms of the disease or disorder are being displayed.

### Salts

Compounds described herein can exist in the form of salts, for example acid addition salts or, in certain cases salts of organic and inorganic bases such as carboxylate, sulfonate and phosphate salts. All such salts are within the scope of this invention, and references to compounds include the salt forms of the compounds as defined herein.

The salts are typically acid addition salts.

The salts for use in the present invention can be synthesized from the parent compound that contains a basic or acidic moiety by conventional chemical methods such as methods described in Pharmaceutical Salts: Properties, Selection, and Use, P. Heinrich Stahl (Editor), Camille G. Wermuth (Editor), ISBN: 3-90639-026-8, Hardcover, 388 pages, August 2002. Generally, such salts can be prepared by reacting the free acid or base forms of these compounds with the appropriate base or acid in water or in an organic solvent, or in a mixture of the two; generally, nonaqueous media such as ether, ethyl acetate, ethanol, isopropanol, or acetonitrile are used.

Acid addition salts may be formed with a wide variety of acids, both inorganic and organic. Examples of acid addition salts falling within the scope of the invention include mono- or di-salts formed with an acid selected from the group consisting of acetic, 2,2-dichloroacetic, adipic, alginic, ascorbic (e.g. L-ascorbic), L-aspartic, benzenesulfonic, benzoic, 4-acetamidobenzoic, butanoic, (+) camphoric, camphor-sulfonic, (+)-(1S)-camphor-10-sulfonic, capric, caproic, caprylic, cinnamic, citric, cyclamic, dodecylsulfuric, ethane-1,2-disulfonic, ethanesulfonic, 2-hydroxyethanesulfonic, formic, fumaric, galactaric, gentisic, glucoheptonic, D-gluconic, glucuronic (e.g. D-glucuronic), glutamic (e.g. L-glutamic), α-oxoglutaric, glycolic, hippuric, hydrohalic acids (e.g. hydrobromic, hydrochloric, hydriodic), isethionic, lactic (e.g. 10 (+)-L-lactic, (±)-DL-lactic), lactobionic, maleic, malic, (−)-L-malic, malonic, (±)-DL-mandelic, methanesulfonic, naphthalene-2-sulfonic, naphthalene-1,5-disulfonic, 1-hydroxy-2-naphthoic, nicotinic, nitric, oleic, orotic, oxalic, palmitic, pamoic, phosphoric, propionic, pyruvic, L-pyroglutamic, salicylic, 4-amino-salicylic, sebacic, stearic, succinic, sulfuric, tannic, (+)-L-tartaric, thiocyanic, p-toluenesulfonic, undecylenic and valeric acids, as well as acylated amino acids and cation exchange resins.

Amine functions in the compounds described herein may form quaternary ammonium salts, for example by reaction with an alkylating agent according to methods well known to the skilled person. Such quaternary ammonium compounds are within the scope of the invention.

The compounds of the invention may exist as mono- or di-salts depending upon the pKa of the acid from which the salt is formed.

The salt forms of the compounds of the invention are typically pharmaceutically acceptable salts, and examples of pharmaceutically acceptable salts are discussed in Berge et al., 1977, “Pharmaceutically Acceptable Salts,” J. Pharm. Sci., Vol. 66, pp. 1-19. However, salts that are not pharmaceutically acceptable may also be prepared as intermediate forms which may then be converted into pharmaceutically acceptable salts. Such non-pharmaceutically acceptable salts forms, which may be useful, for example, in the purification or separation of the compounds of the invention, also form part of the invention.

### Optical Isomers References to the compounds include all optical isomeric forms thereof (e.g. enantiomers, epimers and diastereoisomers), either as individual optical isomers, or mixtures (e.g.

racemic mixtures) or two or more optical isomers, unless the context requires otherwise.

The optical isomers may be characterised and identified by their optical activity (i.e. as + and − isomers, or d and l isomers) or they may be characterised in terms of their absolute stereochemistry using the “R and S” nomenclature developed by Cahn, Ingold and Prelog, see Advanced Organic Chemistry by Jerry March, 4^{th} Edition, John Wiley & Sons, New York, 992, pages 109-114, and see also Cahn, Ingold & Prelog, Angew. Chem. Int. Ed. Engl., 1966, 5, 385-415. Optical isomers can be separated by a number of techniques including chiral chromatography (chromatography on a chiral support) and such techniques are well known to the person skilled in the art. As an alternative to chiral chromatography, optical isomers can be separated by forming diastereoisomeric salts with chiral acids such as (+)-tartaric acid, (−)-pyroglutamic acid, (−)-di-toluoyl-L-tartaric acid, (+)-mandelic acid, (−)-malic acid, and (−)-camphorsulphonic, separating the diastereoisomers by preferential crystallisation, and then dissociating the salts to give the individual enantiomer of the free base.

Where compounds exist as two or more optical isomeric forms, one enantiomer in a pair of enantiomers may exhibit advantages over the other enantiomer, for example, in terms of biological activity. Thus, in certain circumstances, it may be desirable to use as a therapeutic agent only one of a pair of enantiomers, or only one of a plurality of diastereoisomers. Accordingly, the invention includes compositions containing a compound having one or more chiral centres, wherein at least 55% (e.g. at least 60%, 65%, 70%, 75%, 80%, 85%, 90% or 95%) of the compound is present as a single optical isomer (e.g. enantiomer or diastereoisomer).

In one general embodiment, 99% or more (e.g. substantially all) of the total amount of the compound is present as a single optical isomer.

For example, in one embodiment the compound is present as a single diastereoisomer.

The invention also provides mixtures of optical isomers, which may be racemic or non-racemic. Thus, the invention includes:
Compounds which are in the form of a racemic mixture of optical isomers.
Compounds which are in the form of a non-racemic mixture of optical isomers.

### Isotopes

The compounds may contain one or more isotopic substitutions, and a reference to a particular element includes within its scope all isotopes of the element. For example, a reference to hydrogen includes within its scope ¹H, ²H (D), and ³H (T). Similarly, references to carbon and oxygen include within their scope respectively ¹²C, ¹³C and ¹⁴C and ¹⁶O and ¹⁸O.

In an analogous manner, a reference to a particular functional group also includes within its scope isotopic variations, unless the context indicates otherwise. For example, a reference to an alkyl group such as an ethyl group also covers variations in which one or more of the hydrogen atoms in the group is in the form of a deuterium or tritium isotope, e.g. as in an ethyl group in which all five hydrogen atoms are in the deuterium isotopic form (a perdeuteroethyl group).

The isotopes may be radioactive or non-radioactive. The compounds may contain no radioactive isotopes. Such compounds are preferred for therapeutic use. However, the compound may contain one or more radioisotopes.

### Solvates

The compounds may form solvates. Preferred solvates are solvates formed by the incorporation into the solid-state structure (e.g. crystal structure) of the compounds of molecules of a non-toxic pharmaceutically acceptable solvent (referred to below as the solvating solvent). Examples of such solvents include water, alcohols (such as ethanol, isopropanol and butanol) and dimethylsulfoxide. Solvates can be prepared by recrystalising the compounds of the invention with a solvent or mixture of solvents containing the solvating solvent. Whether or not a solvate has been formed in any given instance can be determined by subjecting crystals of the compound to analysis using well known and standard techniques such as thermogravimetric analysis (TGE), differential scanning calorimetry (DSC) and X-ray crystallography. The solvates can be stoichiometric or non-stoichiometric solvates. Particularly preferred solvates are hydrates, and examples of hydrates include hemihydrates, monohydrates and dihydrates.

Accordingly, the invention includes:
Compounds in the form of a solvate.
Compounds wherein the solvate is a hydrate.

For a more detailed discussion of solvates and the methods used to make and characterise them, see Bryn et al., Solid-State Chemistry of Drugs, Second Edition, published by SSCI, Inc of West Lafayette, IN, USA, 1999, ISBN 0-967-06710-3.

Alternatively, rather than existing as a hydrate, the compound of the invention may be anhydrous. Therefore, the invention provides a compound of the invention in an anhydrous form (e.g. anhydrous crystalline form).

### Crystalline and Amorphous Forms

The compounds may exist in a crystalline or non-crystalline (e.g. amorphous) state. Whether or not a compound exists in a crystalline state can readily be determined by standard techniques such as X-ray powder diffraction (XRPD). Crystals and their crystal structures can be characterised using a number of techniques including single crystal X-ray crystallography, X-ray powder diffraction (XRPD), differential scanning calorimetry (DSC) and infra-red spectroscopy, e.g. Fourier Transform infra-red spectroscopy (FTIR). The behaviour of the crystals under conditions of varying humidity can be analysed by gravimetric vapour sorption studies and also by XRPD. Determination of the crystal structure of a compound can be performed by X-ray crystallography which can be carried out according to conventional methods such as those described herein and as described in Fundamentals of Crystallography, C. Giacovazzo, H. L. Monaco, D. Viterbo, F. Scordari, G. Gilli, G. Zanotti and M. Catti, (International Union of Crystallography/Oxford University Press, 1992 ISBN 0-19-855578-4 (p/b), 0-19-85579-2 (h/b)). This technique involves the analysis and interpretation of the X-ray diffraction of single crystal. In an amorphous solid, the three dimensional structure that normally exists in a crystalline form does not exist and the positions of the molecules relative to one another in the amorphous form are essentially random, see for example Hancock et al. J. Pharm. Sci. (1997), 86, 1).

Accordingly, the invention includes:
Compounds in a crystalline form.
A compound which is:
(a) from 50% to 100% crystalline, and more particularly is at least 50% crystalline, or at least 60% crystalline, or at least 70% crystalline, or at least 80% crystalline, or at least 90% crystalline, or at least 95% crystalline, or at least 98% crystalline, or at least 99% crystalline, or at least 99.5% crystalline, or at least 99.9% crystalline, for example 100% crystalline.

A compound which is in an amorphous form.

### Complexes and Clathrates

Also encompassed are complexes (e.g. inclusion complexes or clathrates with compounds such as cyclodextrins, or complexes with metals) of the compounds>

Accordingly, the invention includes compounds in the form of a complex or clathrate.

### Cholinesterase Inhibitors (ChEIs)

Cholinesterase inhibitors (ChEIs), also known as anti-cholinesterase, are compounds that prevent the breakdown of the neurotransmitter acetylcholine or butyrylcholine. This increases the amount of the acetylcholine or butyrylcholine in the synaptic cleft that can bind to muscarinic receptors, nicotinic receptors and others. ChEIs may be used in the treatment of disorders such as Alzheimer's disease and dementia. ChEIs are the standard of care treatment for cognitive and behavioural disturbances in Alzheimer's disease and dementia, but often give insufficient relief of symptoms particularly as the disease progresses. In the invention described herein ChEIs may be co-administered or used alongside a course of treatment with pharmaceutical compositions comprising the compound ethyl 3-(3-oxo-2,8-diazaspiro[4.5]dec-8-yl)-8-azabicyclo[3.2.1]octane-8-carboxylate, or a pharmaceutically acceptable salt thereof. The combined use of such agents may lead to greater improved therapeutic outcomes compared to administration of a ChEI compound or ethyl 3-(3-oxo-2,8-diazaspiro[4.5]dec-8-yl)-8-azabicyclo[3.2.1]octane-8-carboxylate alone.

### Biological Activity and Therapeutic Uses

Compounds described herein have activity as muscarinic M₁ and M₄ receptor agonists. Muscarinic activity can be determined using the Phospho-ERK1/2 assay described in Example A below.

A significant advantage of compounds herein is that they are highly selective for the M₁ and M₄ receptor relative to the M₂ and M₃ receptor subtypes. Compounds herein are neither agonists nor antagonists of the M₂ and M₃ receptor subtypes. For example, whereas compounds of the type described herein have pEC₅₀ values of at least 6 and Eₘₐₓ values of greater than 80 against the M₁ receptor in the functional assay described in Example A, they have pEC₅₀ values of less than 5 and Eₘₐₓ values of less than 20% when tested against the M₂ and M₃ subtypes in the functional assay of Example A.

By virtue of their muscarinic M₁ and M₄ receptor agonist activity, compounds described herein can be used in the treatment of Alzheimer's disease, dementia with Lewy bodies schizophrenia and other psychotic disorders, cognitive disorders and other diseases mediated by the muscarinic M₁ and/or M₄ receptor, and can also be used in the treatment of various types of pain.

Accordingly, further provided is:

A pharmaceutical composition comprising the compound ethyl 3-(3-oxo-2,8-diazaspiro[4.5]dec-8-yl)-8-azabicyclo[3.2.1]octane-8-carboxylate, or a pharmaceutically acceptable salt thereof, for use in the treatment of a cognitive disorder or psychotic disorder. A pharmaceutical composition comprising the compound ethyl 3-(3-oxo-2,8-diazaspiro[4.5]dec-8-yl)-8-azabicyclo[3.2.1]octane-8-carboxylate, or a pharmaceutically acceptable salt thereof, for use in the treatment of a cognitive disorder or psychotic disorder, wherein the cognitive disorder or psychotic disorder comprises, arises from or is associated with a condition selected from cognitive impairment, Mild Cognitive Impairment (MCI), (including amnestic MCI and nonamnestic MCI, and including mild cognitive impairment due to Alzheimer's disease and/or prodromal Alzheimer's disease), frontotemporal dementia, vascular dementia, dementia with Lewy bodies, presenile dementia, senile dementia, Friederich's ataxia, Down's syndrome, Huntington's chorea, hyperkinesia, mania, Tourette's syndrome, Alzheimer's disease (including prodromal Alzheimer's disease and stages 1, 2, and 3 early Alzheimer's disease as defined by the US Food and Drug Administration's “Early Alzheimer's disease: Developing Drugs for Treatment” available at fda.gov/downloads/Drugs/GuidanceComplianceRegulatoryInformation/Guidances/UCM5967 28.pdf), progressive supranuclear palsy, impairment of cognitive functions including attention, orientation, learning disorders, memory (i.e. memory disorders, amnesia, amnesic disorders, transient global amnesia syndrome and age-associated memory impairment) and language function; cognitive impairment as a result of stroke, Huntington's disease, Pick disease, AIDS-related dementia or other dementia states such as multi-infarct dementia, alcoholic dementia, hypothyroidism-related dementia, and dementia associated to other degenerative disorders such as cerebellar atrophy and amyotrophic lateral sclerosis; other acute or sub-acute conditions that may cause cognitive decline such as delirium or depression (pseudodementia states) trauma, head trauma, age related cognitive decline, stroke, neurodegeneration, drug-induced states, neurotoxic agents, age related cognitive impairment, autism related cognitive impairment, Down's syndrome, cognitive deficit related to psychosis, and post-electroconvulsive treatment related cognitive disorders; cognitive disorders due to drug abuse or drug withdrawal including nicotine, cannabis, amphetamine, cocaine, Attention Deficit Hyperactivity Disorder (ADHD) and dyskinetic disorders such as Parkinson's disease, neuroleptic-induced parkinsonism, and tardive dyskinesias, schizophrenia, schizophreniform diseases, psychotic depression, mania, acute mania, paranoid, hallucinogenic and delusional disorders, personality disorders, obsessive compulsive disorders, schizotypal disorders, delusional disorders, psychosis due to malignancy, metabolic disorder, endocrine disease or narcolepsy, psychosis due to drug abuse or drug withdrawal, bipolar disorders and schizo-affective disorder.

A pharmaceutical composition comprising the compound ethyl 3-(3-oxo-2,8-diazaspiro[4.5]dec-8-yl)-8-azabicyclo[3.2.1]octane-8-carbontlate, or a pharmaceutically acceptable salt thereof, for use in the treatment of Alzheimer's disease or dementia.

A pharmaceutical composition comprising the compound ethyl 3-(3-oxo-2,8-diazaspiro[4.5]dec-8-yl)-8-azabicyclo[3.2.1]octane-8-carbontlate, or a pharmaceutically acceptable salt thereof, for use in the treatment of Alzheimer's disease.

A pharmaceutical composition comprising the compound ethyl 3-(3-oxo-2,8-diazaspiro[4.5]dec-8-yl)-8-azabicyclo[3.2.1]octane-8-carbontlate, or a pharmaceutically acceptable salt thereof, for use in the treatment of dementia.

A pharmaceutical composition comprising the compound ethyl 3-(3-oxo-2,8-diazaspiro[4.5]dec-8-yl)-8-azabicyclo[3.2.1]octane-8-carbontlate, or a pharmaceutically acceptable salt thereof, for use in the treatment of dementia with Lewy bodies.

A pharmaceutical composition comprising the compound ethyl 3-(3-oxo-2,8-diazaspiro[4.5]dec-8-yl)-8-azabicyclo[3.2.1]octane-8-carbontlate, or a pharmaceutically acceptable salt thereof, for use in the treatment of schizophrenia.

A pharmaceutical composition comprising the compound ethyl 3-(3-oxo-2,8-diazaspiro[4.5]dec-8-yl)-8-azabicyclo[3.2.1]octane-8-carbontlate, or a pharmaceutically acceptable salt thereof, for use in the treatment of Alzheimer's disease or dementia; wherein the load dose of the compound or pharmaceutically acceptable salt thereof is between 5 to 25 mg.

A pharmaceutical composition comprising the compound ethyl 3-(3-oxo-2,8-diazaspiro[4.5]dec-8-yl)-8-azabicyclo[3.2.1]octane-8-carbontlate, or a pharmaceutically acceptable salt thereof, for use in the treatment of Alzheimer's disease; wherein the load dose of the compound or pharmaceutically acceptable salt thereof is between 5 to 25 mg.

A pharmaceutical composition comprising the compound ethyl 3-(3-oxo-2,8-diazaspiro[4.5]dec-8-yl)-8-azabicyclo[3.2.1]octane-8-carboxylate, or a pharmaceutically acceptable salt thereof, for use in the treatment of dementia; wherein the load dose of the compound or pharmaceutically acceptable salt thereof is between 5 to 25 mg.

A pharmaceutical composition comprising the compound ethyl 3-(3-oxo-2,8-diazaspiro[4.5]dec-8-yl)-8-azabicyclo[3.2.1]octane-8-carboxylate, or a pharmaceutically acceptable salt thereof, for use in the treatment of dementia with Lewy bodies; wherein the load dose of the compound or pharmaceutically acceptable salt thereof is between 5 to 25 mg.

A pharmaceutical composition comprising the compound ethyl 3-(3-oxo-2,8-diazaspiro[4.5]dec-8-yl)-8-azabicyclo[3.2.1]octane-8-carboxylate, or a pharmaceutically acceptable salt thereof, for use in the treatment of schizophrenia; wherein the load dose of the compound or pharmaceutically acceptable salt thereof is between 5 to 25 mg.

A method of treatment of a cognitive disorder in a subject (e.g. a mammalian patient such as a human, e.g. a human in need of such treatment), which method comprises the administration of 5 to 25 mg of ethyl 3-(3-oxo-2,8-diazaspiro[4.5]dec-8-yl)-8-azabicyclo[3.2.1]octane-8-carboxylate, or a pharmaceutically acceptable salt thereof.

A method of treatment of a cognitive disorder in a subject (e.g. a mammalian patient such as a human, e.g. a human in need of such treatment), which method comprises the administration of 5 to 25 mg of ethyl 3-(3-oxo-2,8-diazaspiro[4.5]dec-8-yl)-8-azabicyclo[3.2.1]octane-8-carboxylate, or a pharmaceutically acceptable salt thereof, wherein the cognitive disorder comprises, arises from or is associated with a condition as defined in above.

A method of treatment of a cognitive disorder in a subject (e.g. a mammalian patient such as a human, e.g. a human in need of such treatment), which method comprises the administration of 5 to 25 mg of ethyl 3-(3-oxo-2,8-diazaspiro[4.5]dec-8-yl)-8-azabicyclo[3.2.1]octane-8-carboxylate, or a pharmaceutically acceptable salt thereof, wherein the cognitive disorder arises from or is associated with Alzheimer's disease.

A method of treatment of a cognitive disorder in a subject (e.g. a mammalian patient such as a human, e.g. a human in need of such treatment), which method comprises the administration of 5 to 25 mg of ethyl 3-(3-oxo-2,8-diazaspiro[4.5]dec-8-yl)-8-azabicyclo[3.2.1]octane-8-carboxylate, or a pharmaceutically acceptable salt thereof, wherein the cognitive disorder arises from or is associated with dementia.

A method of treatment of a cognitive disorder in a subject (e.g. a mammalian patient such as a human, e.g. a human in need of such treatment), which method comprises the administration of 5 to 25 mg of ethyl 3-(3-oxo-2,8-diazaspiro[4.5]dec-8-yl)-8-azabicyclo[3.2.1]octane-8-carboxylate, or a pharmaceutically acceptable salt thereof, wherein the cognitive disorder is dementia with Lewy bodies.

A method of treatment of a cognitive disorder in a subject (e.g. a mammalian patient such as a human, e.g. a human in need of such treatment), which method comprises the administration of 5 to 25 mg of ethyl 3-(3-oxo-2,8-diazaspiro[4.5]dec-8-yl)-8-azabicyclo[3.2.1]octane-8-carboxylate, or a pharmaceutically acceptable salt thereof, wherein the cognitive disorder is schizophrenia.

The use of ethyl 3-(3-oxo-2,8-diazaspiro[4.5]dec-8-yl)-8-azabicyclo[3.2.1]octane-8-carboxylate, or a pharmaceutically acceptable salt thereof, for the manufacture of a medicament for the treatment of a cognitive disorder.

The use of ethyl 3-(3-oxo-2,8-diazaspiro[4.5]dec-8-yl)-8-azabicyclo[3.2.1]octane-8-carboxylate, or a pharmaceutically acceptable salt thereof, for the manufacture of a medicament for the treatment of a cognitive disorder, wherein the cognitive disorder comprises, arises from or is associated with a condition as defined in above.

The use of ethyl 3-(3-oxo-2,8-diazaspiro[4.5]dec-8-yl)-8-azabicyclo[3.2.1]octane-8-carboxylate, or a pharmaceutically acceptable salt thereof, for the manufacture of a medicament for the treatment of a cognitive disorder, wherein the cognitive disorder comprises, arises from or is associated with Alzheimer's disease.

The use of ethyl 3-(3-oxo-2,8-diazaspiro[4.5]dec-8-yl)-8-azabicyclo[3.2.1]octane-8-carboxylate, or a pharmaceutically acceptable salt thereof, for the manufacture of a medicament for the treatment of a cognitive disorder, wherein the cognitive disorder comprises, arises from or is associated with dementia.

The use of ethyl 3-(3-oxo-2,8-diazaspiro[4.5]dec-8-yl)-8-azabicyclo[3.2.1]octane-8-carboxylate, or a pharmaceutically acceptable salt thereof, for the manufacture of a medicament for the treatment of a cognitive disorder, wherein the cognitive disorder comprises, arises from or is associated with dementia with Lewy bodies.

The use of ethyl 3-(3-oxo-2,8-diazaspiro[4.5]dec-8-yl)-8-azabicyclo[3.2.1]octane-8-carboxylate, or a pharmaceutically acceptable salt thereof, for the manufacture of a medicament for the treatment of a cognitive disorder, wherein the cognitive disorder comprises, arises from or is associated with schizophrenia.

The pharmaceutical composition comprising the compound ethyl 3-(3-oxo-2,8-diazaspiro[4.5]dec-8-yl)-8-azabicyclo[3.2.1]octane-8-carboxylate, or a pharmaceutically acceptable salt thereof, may be for use in the treatment or lessening the severity of acute, chronic, neuropathic, or inflammatory pain, arthritis, migraine, cluster headaches, trigeminal neuralgia, herpetic neuralgia, general neuralgias, visceral pain, osteoarthritis pain, postherpetic neuralgia, diabetic neuropathy, radicular pain, sciatica, back pain, head or neck pain, severe or intractable pain, nociceptive pain, breakthrough pain, postsurgical pain, or cancer pain.

A method of treatment or lessening the severity of acute, chronic, neuropathic, or inflammatory pain, arthritis, migraine, cluster headaches, trigeminal neuralgia, herpetic neuralgia, general neuralgias, visceral pain, osteoarthritis pain, postherpetic neuralgia, diabetic neuropathy, radicular pain, sciatica, back pain, head or neck pain, severe or intractable pain, nociceptive pain, breakthrough pain, postsurgical pain, or cancer pain, which method comprises the administration of 5 to 25 mg of ethyl 3-(3-oxo-2,8-diazaspiro[4.5]dec-8-yl)-8-azabicyclo[3.2.1]octane-8-carboxylate, or a pharmaceutically acceptable salt thereof.

The pharmaceutical composition comprising the compound ethyl 3-(3-oxo-2,8-diazaspiro[4.5]dec-8-yl)-8-azabicyclo[3.2.1]octane-8-carboxylate, or a pharmaceutically acceptable salt thereof, may be for the treatment of peripheral disorders such as reduction of intra ocular pressure in Glaucoma and treatment of dry eyes and dry mouth including Sjogren's Syndrome.

A method of treatment of peripheral disorders such as reduction of intra ocular pressure in Glaucoma and treatment of dry eyes and dry mouth including Sjogren's Syndrome, which method comprises the administration of 5 to 25 mg of ethyl 3-(3-oxo-2,8-diazaspiro[4.5]dec-8-yl)-8-azabicyclo[3.2.1]octane-8-carboxylate, or a pharmaceutically acceptable salt thereof. The use of ethyl 3-(3-oxo-2,8-diazaspiro[4.5]dec-8-yl)-8-azabicyclo[3.2.1]octane-8-carboxylate, or a pharmaceutically acceptable salt thereof, for the manufacture of a medicament for the treatment or lessening the severity of acute, chronic, neuropathic, or inflammatory pain, arthritis, migraine, cluster headaches, trigeminal neuralgia, herpetic neuralgia, general neuralgias, visceral pain, osteoarthritis pain, postherpetic neuralgia, diabetic neuropathy, radicular pain, sciatica, back pain, head or neck pain, severe or intractable pain, nociceptive pain, breakthrough pain, postsurgical pain, or cancer pain or for the treatment of peripheral disorders such as reduction of intra ocular pressure in Glaucoma and treatment of dry eyes and dry mouth including Sjogren's Syndrome.

The use of ethyl 3-(3-oxo-2,8-diazaspiro[4.5]dec-8-yl)-8-azabicyclo[3.2.1]octane-8-carboxylate, or a pharmaceutically acceptable salt thereof, for the treatment of skin lesions for example due to pemphigus vulgaris, dermatitis herpetiformis, pemphigoid and other blistering skin conditions.

The use of ethyl 3-(3-oxo-2,8-diazaspiro[4.5]dec-8-yl)-8-azabicyclo[3.2.1]octane-8-carboxylate, or a pharmaceutically acceptable salt thereof, for treating, preventing, ameliorating or reversing conditions associated with altered gastro-intestinal function and motility such as functional dyspepsia, irritable bowel syndrome, gastroesophageal acid reflux (GER) and esophageal dysmotility, symptoms of gastroparesis and chronic diarrhea.

The use of ethyl 3-(3-oxo-2,8-diazaspiro[4.5]dec-8-yl)-8-azabicyclo[3.2.1]octane-8-carboxylate, or a pharmaceutically acceptable salt thereof, for the treatment of olfactory dysfunction such as Bosma-Henkin-Christiansen syndrome, chemical poisoning (e.g. selenium and silver), hypopituitarism, Kallmann Syndrome, skull fractures, tumour therapy and underactive thyroid gland.

The use of ethyl 3-(3-oxo-2,8-diazaspiro[4.5]dec-8-yl)-8-azabicyclo[3.2.1]octane-8-carboxylate, or a pharmaceutically acceptable salt thereof, for the treatment of addiction. The use of ethyl 3-(3-oxo-2,8-diazaspiro[4.5]dec-8-yl)-8-azabicyclo[3.2.1]octane-8-carboxylate, or a pharmaceutically acceptable salt thereof, for the treatment of movement disorders such as Parkinson's disease, ADHD, Huntingdon's disease, Tourette's syndrome and other syndromes associated with dopaminergic dysfunction as an underlying pathogenetic factor driving disease.

The use of ethyl 3-(3-oxo-2,8-diazaspiro[4.5]dec-8-yl)-8-azabicyclo[3.2.1]octane-8-carboxylate, or a pharmaceutically acceptable salt thereof, for the treatment of behavioural and psychological symptoms of dementia (BPSD; including agitation, verbal aggressiveness, physical aggressiveness, depression, anxiety, abnormal motor behaviour, elated mood, irritability, apathy, disinhibition, impulsivity. delusions, hallucinations, sleep changes, and appetite changes).

All uses and methods described above relate to pharmaceutical compositions wherein the load dose of ethyl 3-(3-oxo-2,8-diazaspiro[4.5]dec-8-yl)-8-azabicyclo[3.2.1]octane-8-carboxylate, or a pharmaceutically acceptable salt thereof is between 5 to 25 mg. The uses and methods described above also relate to pharmaceutical compositions wherein the composition is co-administered with a standard of care cholinesterase inhibitor, such as Donepezil, which may be administered at a load dose of 10 mg.

### Pharmaceutical Compositions

In the uses and methods described herein, the compound ethyl 3-(3-oxo-2,8-diazaspiro[4.5]dec-8-yl)-8-azabicyclo[3.2.1]octane-8-carboxylate, or a pharmaceutically acceptable salt thereof, is administered as a pharmaceutical composition (e.g. formulation). The composition may comprise one or more pharmaceutically acceptable carriers or excipients.

The composition may be suitable for oral administration. The composition may be a tablet composition. The composition may be a capsule composition.

The pharmaceutically acceptable excipient(s) can be selected from, for example, carriers (e.g. a solid, liquid or semi-solid carrier), adjuvants, diluents (e.g solid diluents such as fillers or bulking agents; and liquid diluents such as solvents and co-solvents), granulating agents, binders, flow aids, coating agents, release-controlling agents (e.g. release retarding or delaying polymers or waxes), binding agents, disintegrants, buffering agents, lubricants, preservatives, anti-fungal and antibacterial agents, antioxidants, buffering agents, tonicity-adjusting agents, thickening agents, flavouring agents, sweeteners, pigments, plasticizers, taste masking agents, stabilisers or any other excipients conventionally used in pharmaceutical compositions.

The term “pharmaceutically acceptable” as used herein means compounds, materials, compositions, and/or dosage forms which are, within the scope of sound medical judgment, suitable for use in contact with the tissues of a subject (e.g. a human subject) without excessive toxicity, irritation, allergic response, or other problem or complication, commensurate with a reasonable benefit/risk ratio. Each excipient must also be “acceptable” in the sense of being compatible with the other ingredients of the formulation.

Pharmaceutical compositions can be formulated in accordance with known techniques, see for example, Remington's Pharmaceutical Sciences, Mack Publishing Company, Easton, PA, USA.

The pharmaceutical compositions can be in any form suitable for oral, parenteral, topical, intranasal, intrabronchial, sublingual, ophthalmic, optic, rectal, intra-vaginal, or transdermal administration.

Pharmaceutical dosage forms suitable for oral administration include tablets (coated or uncoated), capsules (hard or soft shell), caplets, pills, lozenges, syrups, solutions, powders, granules, elixirs and suspensions, sublingual tablets, wafers or patches such as buccal patches.

Tablet compositions can contain a unit dosage of active compound together with an inert diluent or carrier such as a sugar or sugar alcohol, e.g.; lactose, sucrose, sorbitol or mannitol; and/or a non-sugar derived diluent such as sodium carbonate, calcium phosphate, calcium carbonate, or a cellulose or derivative thereof such as microcrystalline cellulose (MCC), methyl cellulose, ethyl cellulose, hydroxypropyl methyl cellulose, and starches such as corn starch. Tablets may also contain such standard ingredients as binding and granulating agents such as polyvinylpyrrolidone, disintegrants (e.g. swellable crosslinked polymers such as crosslinked carboxymethylcellulose), lubricating agents (e.g. stearates), preservatives (e.g. parabens), antioxidants (e.g. BHT), buffering agents (for example phosphate or citrate buffers), and effervescent agents such as citrate/bicarbonate mixtures. Such excipients are well known and do not need to be discussed in detail here.

Tablets may be designed to release the drug either upon contact with stomach fluids (immediate release tablets) or to release in a controlled manner (controlled release tablets) over a prolonged period of time or with a specific region of the GI tract.

The pharmaceutical compositions typically comprise from approximately 1% (w/w) to approximately 95%, preferably% (w/w) active ingredient and from 99% (w/w) to 5% (w/w) of a pharmaceutically acceptable excipient (for example as defined above) or combination of such excipients. Preferably, the compositions comprise from approximately 20% (w/w) to approximately 90% (w/w) active ingredient and from 80% (w/w) to 10% of a pharmaceutically excipient or combination of excipients. The pharmaceutical compositions comprise from approximately 1% to approximately 95%, preferably from approximately 20% to approximately 90%, active ingredient. Pharmaceutical compositions according to the invention may be, for example, in unit dose form, such as in the form of ampoules, vials, suppositories, pre-filled syringes, dragees, powders, tablets or capsules.

Tablets and capsules may contain, for example, 0-20% disintegrants, 0-5% lubricants, 0-5% flow aids and/or 0-99% (w/w) fillers/or bulking agents (depending on drug dose). They may also contain 0-10% (w/w) polymer binders, 0-5% (w/w) antioxidants, 0-5% (w/w) pigments. Slow release tablets would in addition typically contain 0-99% (w/w) release-controlling (e.g. delaying) polymers (depending on dose). The film coats of the tablet or capsule typically contain 0-10% (w/w) polymers, 0-3% (w/w) pigments, and/or 0-2% (w/w) plasticizers.

Parenteral formulations typically contain 0-20% (w/w) buffers, 0-50% (w/w) cosolvents, and/or 0-99% (w/w) Water for Injection (WFI) (depending on dose and if freeze dried). Formulations for intramuscular depots may also contain 0-99% (w/w) oils.

The pharmaceutical formulations may be presented to a patient in “patient packs” containing an entire course of treatment in a single package, usually a blister pack.

The compositions will generally be presented in unit dosage form and, as such, will typically contain sufficient compound to provide a desired level of biological activity. For example, a compositions may contain 5 to 25 mg of ethyl 3-(3-oxo-2,8-diazaspiro[4.5]dec-8-yl)-8-azabicyclo[3.2.1]octane-8-carboxylate, or a pharmaceutically acceptable salt thereof.

The active compound will be administered to a patient in need thereof (for example a human or animal patient) in an amount sufficient to achieve the desired therapeutic effect (effective amount). The precise amounts of compound administered may be determined by a supervising physician in accordance with standard procedures.

### Dosage Regimes

The compounds and compositions described herein may be administered using any dosage regimen suitable to produce the desired effect in a patient. For example, the compounds and compositions may be administered at a frequency and dosage consistent with the methods described in Example C or Example D.

The compounds and compositions may be administered once or more daily. The compounds and compositions may be administered once daily. The compounds and compositions may be administered once daily at a dose of 5-25 mg. The compounds and compositions may be administered once daily at a dose of 5 mg. The compounds and compositions may be administered once daily at a dose of 15 mg. The compounds and compositions may be administered once daily at a dose of 25 mg. The compounds and compositions may be administered at the same time as a course of treatment with a standard of care cholinesterase inhibitor, for example Donepezil. Administration of the compounds and compositions may begin prior to, during or after a course of treatment with a standard of care cholinesterase inhibitor, for example Donepezil. Administration of a standard of care cholinesterase inhibitor, for example Donepezil, may begin prior to, during or after a course of treatment with the compounds and compositions of the invention.

Throughout a course of treatment the compounds and compositions may be administered at an initial daily dose, and then at a second daily dose, and then the treatment may be continued with a third standard daily dose. The initial and second daily dose periods may continue for any number of days, and the third standard daily dose may continue indefinitely or for as long as is desired.

### General Procedures

Commercial reagents were utilized without further purification. Room temperature (rt) refers to approximately 20-27° C. ¹H NMR spectra were recorded at 300 or 400 MHz on either a Bruker or Varian instrument. Chemical shift values are expressed in parts per million (ppm), i.e. (δ)-values. The following abbreviations are used for the multiplicity of the NMR signals: s=singlet, br=broad, d=doublet, t=triplet, q=quartet, quint=quintet, td=triplet of doublets, tt=triplet of triplets, qd=quartet of doublets, ddd=doublet of doublet of doublets, ddt=doublet of doublet of triplets, m=multiplet. Coupling constants are listed as J values, measured in Hz. NMR and mass spectroscopy results were corrected to account for background peaks. Chromatography refers to column chromatography performed using 60-120 mesh silica gel and executed under nitrogen pressure (flash chromatography) conditions.

LCMS experiments were carried out using electrospray conditions under the following conditions:

### LCMS Method

Instruments: Waters Alliance 2795, Waters 2996 PDA detector, Micromass ZQ; Column: Waters X-Bridge C-18, 2.5 micron, 2.1×20 mm or Phenomenex Gemini-NX C-18, 3 micron, 2.0×30 mm; Gradient [time (min)/solvent D in C (%)]: 0.00/2, 0.10/2, 8.40/95, 9.40/95, 9.50/2, 10.00/2; Solvents: solvent C=2.5 L H₂O+2.5 mL ammonia solution; solvent D=2.5 L MeCN+135 mL H₂O+2.5 mL ammonia solution); Injection volume 3 μL; UV detection 230 to 400 nM; column temperature 45° C.; Flow rate 1.5 mL/min.

### Abbreviations

d=day(s)
DCM=dichloromethane
ES=electro spray ionisation
h=hour(s)
HCl=hydrochloric acid
HPLC=high performance liquid chromatography
LC=liquid chromatography
MeOH=methanol
min=minute(s)
MS=mass spectrometry
NMR=nuclear magnetic resonance
rt=room temperature
sat.=saturated
sol.=solution
TLC=thin-layer chromatography
   Prefixes n-, s-, i-, t- and tert- have their usual meanings: normal, secondary, iso, and tertiary.

### Preparation of ethyl 3-(3-oxo-2,8-diazaspiro[4.5]dec-8-yl)-8-azabicyclo[3.2.1]octane-8-carboxylate (Example 2-2)

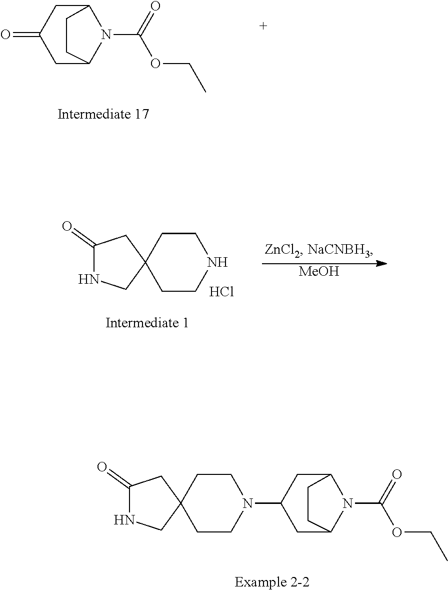 [SMILES: "MeOH" ; CCOC(=O)N1C2CCC1CC(=O)C2 ; O=C1CC2(CCNCC2)CN1 ; Cl ; CCOC(=O)N1C2CCC1CC(N1CCC3(CC1)CNC(=O)C3)C2]

2,8-Diazaspiro[4.5]decan-3-one.HCl (Intermediate 1, CAS: 945892-88-6) and ethyl 3-oxo-8-azabicyclo[3.2.1]octane-8-carboxylate (Intermediate 17, CAS: 32499-64-2) were obtained commercially. 2,8-Diazaspiro[4.5]decan-3-one.HCl (Intermediate 1) (0.40 g, 1.78 mmol) was dissolved in MeOH (3 mL) and treated with K₂CO₃ (0.49 g, 3.55 mmol) in a minimum of water to de-salt. The mixture was concentrated in vacuo. The residue and ethyl 3-oxo-8-azabicyclo[3.2.1]octane-8-carboxylate (Intermediate 17) (0.35 g, 1.78 mmol) were dissolved in MeOH (8 mL) and zinc chloride (0.73 g, 5.33 mmol) was added. The reaction mixture was stirred at 50° C., under a nitrogen atmosphere, for 2 h then cooled to rt and NaCNBH₃ (0.23 g, 3.55 mmol) was added. The reaction mixture was stirred at 50° C. under nitrogen for 16 h. The reaction mixture was cooled to rt and treated with sat. NaHCO₃ sol., the organic solvent was removed in vacuo and the aqueous layer was extracted with DCM (2×10 mL) the organic layers were combined and washed with brine (10 mL) and dried by passing through a Biotage Phase Separator cartridge. The solvents were removed in vacuo, and the residue was purified by column chromatography (normal phase, [Interchim cartridge Puriflash column 15 silica HP-silica 15μ 40 G, 30 mL per min, gradient 0% to 10% MeOH in DCM]) to give ethyl 3-(3-oxo-2,8-diazaspiro[4.5]dec-8-yl)-8-azabicyclo[3.2.1]octane-8-carboxylate Example 2-2 Isomer 1 (16 mg, 2.5%) as an off-white solid and ethyl 3-(3-oxo-2,8-diazaspiro[4.5]dec-8-yl)-8-azabicyclo[3.2.1]octane-8-carboxylate Example 2-2 Isomer 2 (10 mg, 1.7%) as an off-white solid.

### Example 2-2—Isomer 1: ethyl 3-(3-oxo-2,8-d iazaspiro[4.5]dec-8-yl)-8-azabicyclo[3.2.1 ]octane-8-carboxylate

¹H NMR (400 MHz, DMSO-d₆) δ: 1.14 (t, J=7.0 Hz, 3 H), 1.42 - 1.59 (m, 4 H), 1.67-1.92 (m, 9 H), 1.96 (s, 2 H), 2.10-2.45 (m, 4 H), 2.97 (s, 2 H), 3.90 - 4.09 (m, 4 H), 7.49 (br. s., 1 H);

LCMS m/z 336 (M+H)⁺ (ES+), at 2.26 min, UV inactive.

### Example 2-2—Isomer 2: ethyl 3-(3-oxo-2,8-diazaspiro[4.5]dec-8-yl)-8-azabicyclo[3.2.1]octane-8-carboxylate

¹H NMR (400 MHz, DMSO-d₆) δ: 1.15 (t, J=7.0 Hz, 3 H), 1.31-1.56 (m, 6 H), 1.56-1.72 (m, 4 H), 1.72-1.91 (m, 2 H), 1.94 (s, 2 H), 2.20-2.44 (m, 4 H), 2.70-2.75 (m, 1 H), 2.95 (s, 2 H), 4.01 (q, J=7.0 Hz, 2 H), 4.08-4.17 (m, 2 H), 7.44 (s, 1 H);

LCMS m/z 336 (M+H)⁺ (ES+), at 2.14 min, UV inactive.

Individual isolated isomers of Example 2-2 are referred to as Isomer 1 and Isomer 2. Isomer 1 and 2 represent distinct diastereomers of Example 2-2 that have been isolated, characterized and tested individually. While the stereochemical identity of the isomers present is known, stereochemistry is not assigned to either of the two individual isolated species.

Thus, one of Isomer 1 and Isomer 2 is:

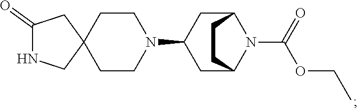

and the other is:

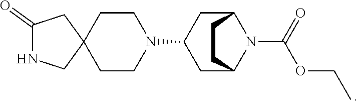

Alternatively, the following synthetic route may be used to prepare Example 2-2 (HTL0018318), and the HCl salt thereof:

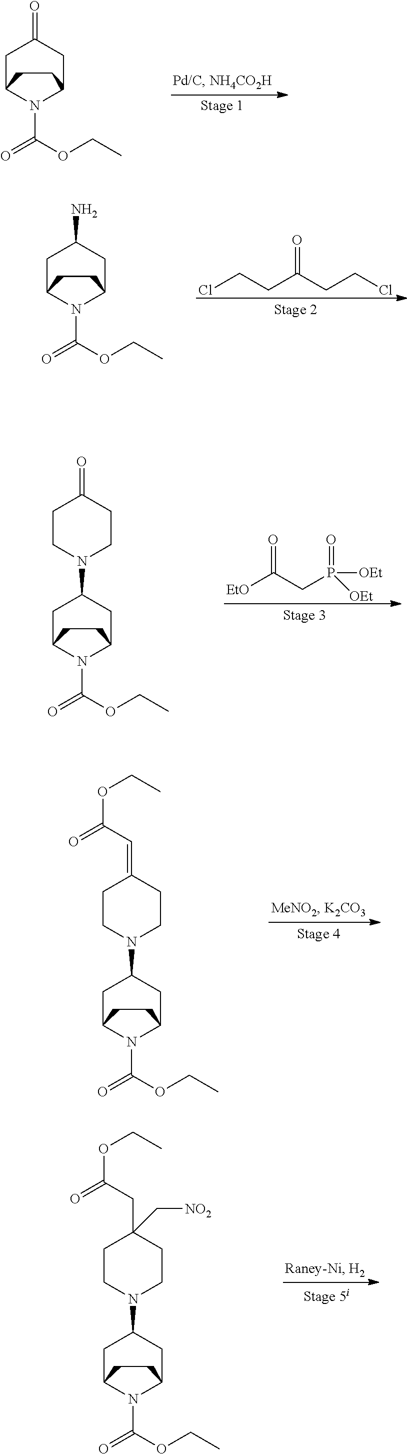

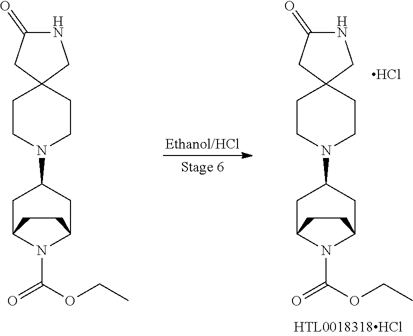 [SMILES: CCOC(=O)N1[C@@H]2CC[C@H]1C[C@@H](N1CCC3(CC1)CNC(=O)C3)C2 ; CCOC(=O)N1[C@@H]2CC[C@H]1C[C@@H](N1CCC3(CC1)CNC(=O)C3)C2 ; Cl]

### Stage 1

Dissolve N-(ethoxycarbonyl) nortropinone (1.0 wt, 1 eq) in methanol (9.0 vol). In a separate vessel dissolve ammonium formate in water (1.0 vol). Add the ammonium formate solution to the starting material solution. Degas the reaction mixture via vacuum/nitrogen cycles. Under nitrogen charge 10% Pd/C (0.2 wt including water) to the reaction. Rapid gas evolution will be observed for the initial stage of the reaction. Maintain the reaction at 15 to 25° C. throughout. Monitor the reaction progress by ¹H NMR², expected reaction time 36 to 48 h. Target <3 mol % N-(ethoxycarbonyl) nortropinone remaining. Dilute the reaction mixture with methanol/water (9:1, 5 vol) and mix for at least 20 minutes. Filter the reaction mixture (glass microfibre) and wash the filter cake with methanol/water (9:1, 2×5 vol). Concentrate the combined filtrates at up to 45° C. on a rotary evaporator until no more methanol is removed leaving an aqueous solution. Charge dichloromethane (10 vol) and 1M HCl (10 vol) to the residue. Ensure the pH is 3 and add more 1M HCl if required. Mix for at least 10 minutes at 15 to 25° C. and separate the phases. Return the organic phase to the flask and extract with 1M HCl (5 vol), mixing for at least 10 minutes then separate the phases. Combine the two acidic aqueous extracts and charge dichloromethane (10 vol) followed by 4M sodium hydroxide (4 vol) whilst maintaining the temperature at 15 to 25° C. Check the pH is 12 and add additional 4M sodium hydroxide if required. Stir the mixture for at least 10 minutes and separate the phases. Return the aqueous phase to the flask and charge dichloromethane (10 vol). Mix the phases for at least 10 minutes at 15 to 25° C. and separate the phases. Combine the two organic extracts and charge water (5 vol). Mix the phases for at least 10 minutes and separate the phases. Return the organic phase to the flask and charge saturated brine (5 vol). Mix for at least 10 minutes at 15 to 25° C. and separate the phases. To the organic phase charge sodium sulfate (1 wt) and mix for at least 10 minutes at 15 to 25° C. Filter the slurry and wash the filter cake with dichloromethane (2 vol). Combine the filtrates and concentrate at up to 40° C. on a rotary evaporator to obtain the product as an off white solid (80 to 95% th).

### Stage 2

Charge stage 1 (1.0 wt), sodium carbonate (0.65 wt, 1.22 eq), ethanol (26.5 vol) and water (13.5 vol) to a flask and heat to reflux (ca. 80° C.). Dissolve 1,5-dichloropentanone (0.82 wt, corrected for assay) in ethanol (4 vol). Add the 1,5-dichloropentanone solution to the refluxing reaction mixture at a steady rate over at least 2 h followed by a line rinse with ethanol (0.5 vol). Monitor the reaction by TLC analysis. The reaction is considered complete when the starting material is absent. Cool the reaction mixture to ≤45° C. and transfer to a rotary evaporator for concentration at up to 45° C. Continue the concentration until all the ethanol is removed leaving an aqueous solution of the product. Charge dichloromethane (10 vol) and the product solution to a flask and mix at 15 to 25° C. Add 1M hydrochloric acid (10 vol), (care possible foaming/gas evolution) whilst maintaining the temperature at 15 to 25° C. If the pH is >3 add additional 1M hydrochloric acid as required. Mix for at least 10 minutes at 15 to 25° C. and separate the phases. Return the organic phase to the flask and charge 1M hydrochloric acid (5 vol) and mix for at least 10 minutes at 15 to 25° C. then separate the phases. Combine the acidic aqueous extracts and add dichloromethane (10 vol) and mix for at least 10 minutes at 15 to 25° C. then separate the phases. Perform a second wash with dichloromethane (10 vol) and mix for at least 10 minutes at 15 to 25° C. then separate the phases. Charge dichloromethane (10 vol) and add 4M sodium hydroxide (4 vol) maintaining the temperature at ≤25° C. Check the pH is ≥12 and if required add additional 4M sodium hydroxide. Stir the mixture for at least 10 minutes and separate the phases. Return the aqueous phase to the flask and charge dichloromethane (10 vol) mix the phases at 15 to 25° C. for at least 10 minutes and separate the phases. Combine the organic extracts and add water (5 vol) and mix for at least 10 minutes at 15 to 25° C. and separate the phases. Charge saturated brine (5 vol) to the flask and wash the organic phase at 15 to 25° C. for at least 10 minutes and separate the phases. Charge sodium sulfate (1 wt) to the flask and mix for at least 10 minutes, filter and wash the filter cake with dichloromethane (2 vol).

Concentrate the filtrates at up to 35° C. on a rotary evaporator to obtain the product as an orange oil in a yield of 80 to 100% th.

### Stage 3

To a suitably sized vessel under nitrogen, charge sodium hydride, 60% dispersion in mineral oil (0.15 wt) and tetrahydrofuran (10 vol). Cool the suspension to 0 to 5° C. To a separate vessel charge triethyl phosphonoacetate (0.85 vol) and tetrahydrofuran (4 vol) and mix at 15 to 25° C. to form a solution. Charge the triethyl phosphonoacetate/THF solution to the sodium hydride suspension over at least 1 hour maintaining the temperature at 0 to 5° C. (Care: hydrogen gas evolution). Perform a line rinse with tetrahydrofuran (1 vol). Stir the resulting solution at 0 to 5° C. for at least 30 minutes. To a separate vessel charge HTL0018318 piperidone (1.0 wt) and tetrahydrofuran (4 vol) and mix at 15 to 25° C. to form a solution. Charge the HTL0018318 piperidone solution to the reaction over at least 1 h maintaining the reaction at 0 to 5° C. Perform a line rinse with tetrahydrofuran (0.5 vol). Continue to mix the reaction at 0 to 5° C. for at least 1 h then warm to 15 to 25° C. (note: during this stir period a gel forms which requires good mixing). Age the reaction for at least 1 h at 15 to 25° C. and then perform an IPC by 1H NMR. Once the reaction meets the pass criterion of ≤1.0 mol % HTL0018318 piperidone remaining charge purified water (2 vol) to the reaction and concentrate the resulting solution on a rotary evaporator at up to 45° C., To the residue, charge iso-propyl acetate (10 vol) and adjust the temperature to 15 to 25° C. Add 1M HCl (10 vol) maintaining the temperature at 15 to 25° C. If the pH is >3.0, add additional 1M HCl as required then mix for at least 20 minutes and then separate the phases. Mix the organic phase with 1M HCl (5 vol) for at least 20 minutes at 15 to 25° C. and then separate the phases. Combine the acidic aqueous extracts and add iso-propyl acetate (10 vol), mix the phases for at least 15 minutes at 15 to 25° C. and then separate the phases. Mix the acidic aqueous phase with iso-propyl acetate (10 vol) and cool the flask contents to 0 to 10° C. Add 4M sodium hydroxide solution (4 vol) at ≤20° C. then check the pH is ≥12 and if required further adjust the pH to 12 to 14 with 4M sodium hydroxide solution. Adjust the temperature of the mixture to 15 to 25° C. and age for at least 20 minutes then separate the phases. Return the aqueous phase to the vessel and charge iso-propyl acetate (10 vol). Check the pH is >12 and adjust to pH 12 to 14 with 4M sodium hydroxide solution if required. Mix for at least 20 minutes at 15 to 25° C. then separate the phases. Combine the organic extracts and charge purified water (5 vol) and mix for at least 15 minutes at 15 to 25° C. then separate the phases. Charge saturated brine solution (5 vol) and mix for at least 15 minutes at 15 to 25° C. then separate the phases. Dry the organic phase over sodium sulphate (1 wt) mixing for at least 15 minutes at 15 to 25° C. Filter the slurry and wash the filter cake with iso-propyl acetate (3 vol). Concentrate the combined filtrates at up to 45° C. on a rotary evaporator until a residue is obtained. Charge n-heptane (5 vol) and re-concentrate at up to 45° C. Analyse the residue to determine the iso-propyl acetate content by 1H NMR analysis. If the iso-propyl acetate content is >1.0% w/w , perform additional n-heptane azeotropes as required. The product will be obtained as a solid in a yield of 75 to 100% th, 94 to 125% w/w yield.

### Stage 4

To a suitable vessel charge HTL0018318 unsaturated ester (1.0wt) and DMSO (4.5 vol). Heat the resulting mixture to 35 to 45° C. Charge nitromethane (0.46 vol) followed by a line rinse with DMSO (0.5 vol) whilst maintaining the temperature at 35 to 45° C. Charge potassium carbonate (0.2 wt) to the reaction mixture and then heat the flask contents to 80 to 90° C. Continue to stir the reaction mixture at 80 to 90° C., remove a sample after at least 2 hours and at appropriate intervals thereafter if required (until deemed complete) and analyse by 1H NMR analysis (expected reaction time 2 to 4 h; pass criterion: ≥99.0% conversion). Cool the reaction mixture to 15 to 20° C. Charge dichloromethane (5 vol) followed by 13% w/w sodium chloride solution (5 vol) whilst maintaining the temperature at ≤30° C. Stir the resulting biphasic mixture for at least 20 minutes at 15 to 25° C. and then separate the phases. Extract the aqueous phase with dichloromethane (3×10 vol) mixing each wash for at least 20 minutes at 15 to 25° C. before performing the phase separations. Combine the organic extracts and wash with purified water (10 vol) for at least 20 minutes at 15 to 25° C. then separate the phases. Wash the organic phase with saturated brine solution (10 vol) for at least 20 minutes at 15 to 25° C. and separate the phases. To the organic phase charge sodium sulfate (1 wt) and mix for at least 20 minutes at 15 to 25° C., filter and wash the filter cake with dichloromethane (3 vol). Concentrate the combined filtrates at up to 35° C. on a rotary evaporator.

### Purification

To a suitably sized sintered funnel, charge silica gel (RM0011, 6 wt) and wash with ethyl acetate (10 vol). Dissolve the crude product in ethyl acetate (1 vol) and load onto the silica. Elute the product from the silica using ethyl acetate (10 vol) fractions as required. Assess the fractions for product content by TLC analysis. Combine all the product containing fractions and concentrate at up to 40° C. on a rotary evaporator until the ethyl acetate content is s 10% w/w as analysed by 1H NMR to give the product as an oil in a yield of 80 to 100% th.

### Stage 5

To a suitable vessel charge HTL0018318 nitro ester (1.0 wt) and absolute ethanol (10 vol) and mix to form a solution at 15 to 25° C. Add 2M ammonia in ethanol (2 vol) to the resulting solution. To a suitable pressure vessel charge a suspension of Raney nickel 2800 slurry in water (0.2 wt). Under a nitrogen atmosphere add the ethanol solution to the pressure vessel. Perform a line rinse with absolute ethanol (2 vol). Degas the vessel contents using nitrogen/vacuum cycles. Pressurise the vessel to 1 to 1.5 barg with hydrogen and evacuate the vessel three times. Increase the hydrogen pressure to 3 barg and heat the contents to 45 to 55° C. (Target 50° C.). Once stable increase the pressure to 5barg. Mix the reaction at 45 to 55° C. for at least 24 hrs and then sample for IPC analysis by 1H NMR. Once the reaction meets the pass criteria of ≤0.5% stage 4 and ≤0.5% intermediate remove the contents from the pressure vessel and rinse out with absolute ethanol (2×2 vol). Filter the reaction mixture under nitrogen and wash the filter cake with absolute ethanol (4 vol). Concentrate the filtrates at up to 40° C. under vacuum on a rotary evaporator to a residue. To the residue, charge dichloromethane (10 vol) and 1M HCl (10 vol) then check the pH is ≤3 and adjust as necessary with additional 1M hydrochloric acid. Adjust the temperature to 15 to 25° C. and mix for at least 10 minutes, and separate the phases. Mix the organic phase with 1M HCl (5 vol), check the pH is ≤3 and adjust as necessary with additional 1M hydrochloric acid then adjust the temperature to 15 to 25° C. and mix for at least 10 minutes, then allow to separate for at least 10 minutes at 15 to 25° C. and then separate the phases. Combine the acidic aqueous extracts and add dichloromethane (10 vol), mix the phases for at least 10 minutes at 15 to 25° C. and then separate the phases. Mix the acidic aqueous phase with dichloromethane (10 vol) and add 4M sodium hydroxide solution (4 vol) at ≤25° C. Check the pH is ≥12 and if required further adjust the pH to s 12 to 14 with 4M sodium hydroxide solution. Adjust the temperature of the mixture to 15 to 25° C. and age for at least 10 minutes, and separate the phases. Return the aqueous phase to the vessel and charge dichloromethane (5 vol) and mix at 15 to 25° C. for at least 10 minutes, then separate the phases. Combine the organic extracts and charge purified water (5 vol) and mix for at least 10 minutes at 15 to 25° C. then separate the phases. Charge saturated brine solution (5 vol) and mix for at least 10 minutes at 15 to 25° C. then separate the phases. Dry the organic phase over sodium sulphate (1 wt) mixing for at least 10 minutes at 15 to 25° C. Filter the slurry and wash the filter cake with dichloromethane (2 vol). Charge activated charcoal (RM0192, 0.1 wt) to the combined filtrates. Adjust the temperature to 15 to 25° C. and stir for at least 3 h. Filter the reaction mixture and wash the filter cake with dichloromethane (4 vol). Concentrate the filtrates at up to 35° C. on a rotary evaporator to a residue until the dichloromethane content is s 1.0% w/w by 1H NMR. The product will be obtained as a solid in a yield of 70 to 100% th, 57 to 82% w/w yield.

### Stage 6

Prepare a 1.8M solution of hydrogen chloride in ethanol by bubbling hydrogen chloride gas (0.2 wt) through absolute ethanol (RM0067, 3 vol) and filter to clarify. Dissolve HTL0018318 free base (1.0 wt) in absolute ethanol (10 vol) at up to 40° C., clarify the resulting solution and perform a filter wash with absolute ethanol (0.5 vol). Add 1.8M hydrogen chloride in ethanol (2 vol) to the HTL0018318 free base solution whilst maintaining the temperature at ≤30° C. Age the suspension formed for at least 30 minutes and then cool to 0 to 5° C. Age the suspension for 2 to 4 h at 0 to 5° C. and then filter the suspension. Wash the filter cake with clarified absolute ethanol at 0 to 5° C. (2×1 vol). Pull the filter cake dry on the filter for at least 4 h under nitrogen. Return the filter cake to a clean flask and charge clarified absolute ethanol (13 vol). Heat the slurry to reflux (75 to 85° C.) and maintain for up to 30 minutes to obtain solution. If solution is not obtained, charge clarified absolute ethanol (up to 4 vol) in portions until dissolution is obtained. Cool the resulting solution to 15 to 25° C. over at least 12 h. Age the slurry at 15 to 25° C. for 2 to 4 h. Filter the slurry under nitrogen and wash the filter cake with clarified absolute ethanol (2×1 vol) at 15 to 25° C. Pull the filter cake dry on the filter under nitrogen until the ethanol content is <0.2% w/w by 1H NMR analysis. Sample the filter cake and submit for chemical purity testing. Sieve the product through a 1.4 mm mesh sieve. A second crystallisation may be required following the same procedure as described above. The product will be obtained as a white to off-white solid in a yield of 70 to 90% th and 78 to 100% w/w. following salt formation and a single crystallization. If a second crystallisation is performed the product is expected to be obtained as a white to off-white solid in a yield of 60 to 85% th and 67 to 94% w/w .

### BIOLOGICAL ACTIVITY

### Example A

### Phospho-ERK1/2 Assays

Functional assays were performed using the Alphascreen Surefire phospho-ERK1/2 assay (Crouch & Osmond, Comb. Chem. High Throughput Screen, 2008). ERK1/2 phosphorylation is a downstream consequence of both Gq/11 and Gi/o protein coupled receptor activation, making it highly suitable for the assessment of M₁, M₃ (Gq/11 coupled) and M₂, M₄ receptors (Gi/o coupled), rather than using different assay formats for different receptor subtypes. CHO cells stably expressing the human muscarinic M₁, M₂, M₃ or M₄ receptor were plated (25K/well) onto 96-well tissue culture plates in MEM-alpha+10% dialysed FBS. Once adhered, cells were serum-starved overnight. Agonist stimulation was performed by the addition of 5 μL agonist to the cells for 5 min (37° C.). Media was removed and 50 μL of lysis buffer added. After 15 min, a 4 μL sample was transferred to 384-well plate and 7 μL of detection mixture added. Plates were incubated for 2 h with gentle agitation in the dark and then read on a PHERAstar plate reader. pEC₅₀ and Eₘₐₓ figures were calculated from the resulting data for each receptor subtype. The results are set out in Table 1 below.

TABLE 1

| | Muscarinic Activity | | | | | | | |
|---|---|---|---|---|---|---|---|---|
| | pEC₅₀ M₁ | | pEC₅₀ M₂ | | pEC₅₀ M₃ | | pEC₅₀ M₄ | |
| | (% Emax | | (% Emax | | (% Emax | | (% Emax | |
| Ex. No. | cf. ACh) | | cf. ACh) | | cf. ACh) | | cf. ACh) | |
| ACh | 8.3 | (102) | 7.8 | (105) | 8.1 | (115) | 8.1 | (110) |
| 2-2 Isomer 1 | 7.1 | (99) | <4.7 | (0) | <4.7 | (17) | 6.7 | (53) |
| 2-2 Isomer 2 | 7.9 | (86) | <4.7 | (0) | <4.7 | (0) | 7.6 | (31) |

### Example B

### Passive Avoidance

Studies were carried out as described previously by Foley et al., (2004) Neuropsychopharmacology. In the passive avoidance task scopolamine administration (1 mg/kg, i.p.) at 6 hours following training rendered animals amnesic of the paradigm. A dose range of 3, 10, and 30 mg/kg (po) free base, administered 90 minutes prior to the training period via oral gavage, was examined.

Example 2-2 Isomer 1 was found to reverse scopolamine-induced amnesia of the paradigm in a dose-dependent manner, with an approximate ED₅₀ of ca. 10 mg/kg (po). The effect of 30 mg/kg was similar to that produced by the cholinesterase inhibitor donepezil (0.1 mg/kg, ip) which served as a positive control (FIG. 1).

### In-Human Studies

In the studies of Example C and Example D, compound 2-2 is referred to as HTL0018318.

### Example C

### Trial Design and Subjects

This was a randomized, fixed-sequence, double-blind, placebo-controlled trial investigating multiple doses of 15 mg (12 subjects; 8 active and 4 placebo) and 25 mg (24 subjects; 16 active and 8 placebo) HTL0018318 given with and without donepezil at steady state in healthy elderly subjects. Open label donepezil (taken in the evening) was up titrated to steady state plasma concentrations by administering 5 mg donepezil for 5 consecutive days once daily, followed by 10 mg donepezil (therapeutic dose level) for 15 consecutive days once daily. Subsequently the donepezil treatment was combined with HTL0018318 or placebo dosed daily for 5 consecutive days (taken in the morning). This was followed by a wash-out period of 20 days and subsequent administration of HTL0018318 or placebo alone, daily for 5 consecutive days, was given at the same dose as previously administered in combination (FIG. 2). As it was expected that some subjects would withdraw from study participation due to side effects of donepezil during the donepezil run-in period, this treatment sequence prevented unnecessary exposure to HTL0018318 in subjects who had not previously completed the donepezil run-in phase. During the 2 periods in which HTL0018318 or placebo were administered, safety and PK measurements were performed daily. The study was run in three cohorts to allow within study modification of the dose of HTL0018318 in the event that an unexpected interaction occurred.

Elderly subjects aged 65-80 years (inclusive) participated in the study. Subjects were eligible if in good health, with a maximum resting blood pressure of up to 150/90 mm Hg and a heart rate between 45-100 bpm at screening. Main exclusion criteria were current or past history of any illness interfering with the study objectives, the use of antihypertensive drugs, products that influence CYP3A4 or CYP2D6 and clinically relevant abnormalities on a 24-hour Holter ECG.

### Materials

HTL0018318 was administered orally as an aqueous solution in 100 ml. Water was used as placebo. To mask the difference in taste between HTL0018318 and placebo, a peppermint strip (Listerine) was administered one minute before and after the administration of the oral solution. In humans, the time to the maximum observed plasma HTL0018318 concentration (Tₘₐₓ) was 1-2 hours and a half-life of approximately 16 hours, which permits once daily dosing (Bakker C, Tasker T, Liptrot J, Hart E P, Klaassen E. S, Doll R. J, et al. Alzheimer's Research & Therapy. 2020; (submitted); Bakker C, Tasker T, Liptrot J, Hart E P, Klaassen E S, Prins S, et al. Br J Clin Pharmacol. 2020; (submitted)). Steady state was reached after 2 or 3 daily doses (Bakker C, Tasker T, Liptrot J, Hart E P, Klaassen E. S, Doll R. J, et al. Alzheimer's Research & Therapy. 2020; (submitted)). Donepezil (manufactured by Aliud Pharma GmbH, Laichingen, Germany) was administered as 5 mg tablets. Donepezil has a Tₘₐₓ of 3-4 hours and a half-life of approximately 70 hours.

### Safety and Tolerability Assessments

A detailed overview of the timing of all measurements is provided in Table 7. AEs were summarised per treatment (i.e. 15 mg HTL0018318, 25 mg HTL0018318 or placebo) and per study phase (i.e. donepezil alone, HTL0018318/placebo in combination with donepezil and HTL0018318/placebo alone). The AEs that were reported when donepezil was administered alone were summarised per treatment given after this phase (e.g. AEs reported when donepezil was administered alone by subjects that were to receive 15 mg HTL0018318 later on during the study). A subset of AEs was created that have a possible relation to increased cholinergic stimulation, being: hyperhidrosis, salivary hypersecretion, hypertension, tachycardia, bradycardia, nausea, diarrhoea, vomiting, constipation, insomnia, dizziness, muscle spasms, hot flush and cold sweat.

Systolic and diastolic blood pressure and pulse rate, all measured in supine and standing position, safety laboratory, electrocardiogram (ECG), and 24-hour Holter ECG were performed.

Saliva production was assessed by measuring the change in weight of three Salivette® dental rolls put into the oral cavity for 3 minutes. Pulmonary function was measured using the Spirostik (Accuramed), a PC-based open spirometry system. Subjective feelings were assessed using the visual analogue scale (VAS) according to Bond & Lader (Bond A, Lader M. British Journal of Medical Psychology. 1974; 47(3):211-8.) and a VAS for nausea (0-100 mm). The Leeds Sleep Evaluation Questionnaire (LSEQ) was used to monitor changes in ease of initiating sleep, quality of sleep, ease of waking, and behaviour following wakefulness (Parrott A C, Hindmarch I. Psychopharmacology (Berl). 1980; 71(2):173-9).

### Pharmacokinetic Assessments

To determine plasma donepezil concentrations, blood samples were collected after the 5th donepezil administration and as shown in FIG. 3. The time point 15 hours post donepezil dose corresponds with the time point immediately prior to HTL0018318 dosing.

To determine plasma HTL0018318 concentrations, blood samples were frequently taken on days when the first and fifth dose of HTL0018318 in combination with and without donepezil was administered. On the days between, only pre-dose PK samples were taken. The last PK blood sample was taken between 7-14 days after the last HTL0018318 dose (Table 7).

To estimate HTL0018318 urine concentrations, all urine was collected within 24 hours after the first dose, and within 72 hours after the last dose of HTL0018318 in combination with and without donepezil.

PK parameters included in the analysis were the maximum observed plasma concentration (Cₘₐₓ), Tₘₐₓ, plasma concentration 24 hr post-dose (Cₘᵢₙ), area under the plasma-concentration-time curve (AUC) from zero to 24 hr post dose (AUC₀₋₂₄), from zero to the end of the dose interval (AUC₀₋ₜₐᵤ), from zero to infinity (AUC_{0-inf}), apparent elimination half-life (t½), apparent oral clearance (CL/F), renal clearance (CLr) and percentage of dose excreted renally as unchanged drug (Ae %), and coefficient of variation (% CV).

### Statistical Analysis

A sample size was chosen typical of drug-drug interaction studies (Dai D, Yang H, Nabhan S, Liu H, Hickman D, Liu G, et al. European journal of clinical pharmacology. 2019; 75(8):1099-108; Sun L, McDonnell D, Yu M, Kumar V, von Moltke L. Clinical drug investigation. 2019; 39(5):477-84; Maekawa Y, Furuie H, Kato M, Myobatake Y, Kamiyama E, Watanabe A, et al. Clinical drug investigation. 2019; 39(10):967-78), the study was not statistically powered. The safety and tolerability assessments of saliva measurement, pulmonary function test, VAS Bond&Lader, VAS nausea, LSEQ and vital signs measured during the periods that HTL0018318 or placebo were administered in combination with and without donepezil were subjected to exploratory analysis. To this end a mixed model analysis of variance was used with treatment, period, time, treatment by period, period by time, treatment by time and treatment by period by time as fixed factors. Subject, subject by period and subject by time were random factors and the pre-HTL0018318 baseline measurement per period was a covariate. In these analysis models, all means are estimated (least square means, LSM). Statistical analysis was conducted with SAS 9.4 for Windows (SAS Institute Inc., Cary, NC, USA). The following contrasts were calculated: HTL0018318 alone vs placebo alone, HTL0018318+donepezil vs placebo+donepezil, HTL0018318+donepezil vs HTL0018318 alone. Analyses were performed for 15 mg and 25 mg HTL0018318 dose levels separately.

The effect of HTL0018318 on the PK of donepezil was analysed by comparing the plasma donepezil concentrations sampled pre-dose, 4 h and 15 h after the 20^{th} donepezil dose (i.e. prior to HTL0018318 or placebo) with the plasma donepezil concentrations at the same times of the 21^{st} and 24^{th} donepezil dose. The 21^{st} and 24^{th} donepezil dose were administered after the first and fourth HTL0018318 administration, respectively.

The effects of donepezil on the PK of HTL0018318 were assessed by comparing the HTL0018318 Cₘₐₓ, Tₘₐₓ and AUC₀₋₂₄ after the first dose of HTL0018318 in combination with donepezil with the same parameters when HTL0018318 was administered without donepezil. Also, the HTL0018318 Cₘₐₓ, AUC₀₋ₜₐᵤ, Tₘₐₓ and Cₘᵢₙ after the last dose of HTL0018318 in combination with donepezil was compared with the same parameters when HTL0018318 was administered without donepezil. For these calculations, data of 15 mg and 25 mg HTL0018318 were grouped together.

The ratio of each above-mentioned parameter with and without donepezil co-dosing was calculated and the 90% CI of the geometric mean was assessed.

The degree of accumulation of exposure to HTL0018318 over the study period was assessed by calculating the ratio of AUC₀₋ₜₐᵤ following repeat dosing to the AUC₀₋ₜₐᵤ following the first dose. To assess the effect of donepezil co-administration on accumulation, these ratios calculated during the treatment period with co-administration of donepezil and without co-administration of donepezil were compared.

All PK analyses were performed in Phoenix 64 build 6.4.0.768 using WinNonlin 6.4. Statistical analysis was performed in R version 3.3.1 (2016-06-21) Copyright (C) 2016 The R Foundation for Statistical Computing (Platform: x86_64-w64-mingw32/x64 64-bit).

### Results

### Subjects

In total 42 subjects enrolled in this study, of whom three subjects withdrew due to side effects of donepezil and three subjects were withdrawn upon re-evaluation of eligibility, all prior to co-administration of HTL0018318. The remaining 36 subjects were randomized to placebo (n=12), 15 mg HTL0018318 (n=8), or 25 mg HTL0018318 (n=16) (Table 2).

TABLE 2
Demographics

| | Placebo | | HTL0018318 | | HTL0018318 | |
|---|---|---|---|---|---|---|
| | (n = 12) | | 15 mg (n = 8) | | 25 mg (n = 16) | |
| Age (years) | | | | | | |
| Mean (range) | 68.5 | (65-71) | 70.4 | (67-73) | 68.3 | (65-75) |
| Weight (kg) | | | | | | |
| Mean (range) | 70.7 | (55, 89.1) | 73.9 | (64.05, 81.8) | 74.6 | (60.2, 86.5) |
| BMI (kg/m²) | | | | | | |
| Mean | 25.2 | (20.7, 31.2) | 25.8 | (22, 27.6) | 24.8 | (19.9, 33.6) |
| (Min, Max) | | | | | | |
| Sex | | | | | | |
| Female | 6 | (50%) | 4 | (50%) | 6 | (38%) |
| Male | 6 | (50%) | 4 | (50%) | 10 | (63%) |

| | | | | | |
|---|---|---|---|---|---|
| CYP2D6 | | | | | |
| predicted | | | | | |
| phenotype | | | | | |
| IM | Genotyping | 1 | (12%) | 2 | (13%) |
| EM | not | 7 | (88%) | 13 | (81%) |

| | | | | |
|---|---|---|---|---|
| PM | performed | 0 | 1 | (6%) |

EM = Extensive metabolizer;
IM = Intermediate metabolizer;
PM = Poor metabolizer

After the first dose of the HTL0018318/placebo in combination with donepezil five subjects dropped out due to a presumed viral gastro-enteritis (n=2 on placebo, n=3 on 25 mg HTL0018318) and one subject missed the fifth placebo dose due to this presumed viral gastro-enteritis. Another two subjects were withdrawn because of non-study drug related abnormal laboratory results after the washout period prior to first administration of HTL0018318/placebo without donepezil. In total 28 subjects completed the study.

### Safety and Tolerability

No clinically significant changes, related to treatment, were seen in any of the laboratory tests, ECG assessment and 24-hour Holter ECG results.

There were no significant changes in standing systolic blood pressure, supine and standing diastolic blood pressure, standing-supine blood pressure or standing pulse rate after HTL0018318 in combination with donepezil compared with HTL0018318 alone. Only effects on supine systolic blood pressure, supine pulse rate and standing-supine pulse rate were observed.

Supine systolic blood pressure was significantly lower after administration of 25 mg HTL0018318 without donepezil (118 mm Hg), but not after administration of 15 mg, compared with 25 mg and 15 mg HTL0018318 respectively in combination with donepezil (120 mm Hg, mean difference of 1.6 mm Hg, 95% CI [-3.1;-0.1], p=0.0378). After placebo without donepezil (118 mm Hg) the supine systolic blood pressure was significantly lower compared with placebo in combination with donepezil (120 mm Hg, mean difference of 1.7 mm Hg, 95% CI [−3.2; −0.2], p=0.0242). Administration of HTL0018318 (at both 15 mg and 25 mg) showed no significant effects on supine systolic blood pressure when compared with placebo either in combination with or without donepezil.

Supine pulse rate was significantly lower after administration of 15 mg and 25 mg HTL0018318 in combination with donepezil compared with HTL0018318 alone (15 mg HTL0018318 in combination with donepezil (64 bpm) vs 15 mg HTL0018318 without donepezil (67 bpm): mean difference of 3.3 bpm, 95% CI [1.5; 5.1], p=0.0009; 25 mg HTL0018318 in combination with donepezil (64 bpm) vs 25 mg HTL0018318 without donepezil (66 bpm): mean difference of 1.5 bpm, 95% CI [0.2; 2.9], p=0.0302). Administration of HTL0018318 (both 15 mg and 25 mg) showed no significant effects on supine pulse rate when compared with placebo either in combination with or without donepezil.

The change in pulse rate when standing from the pulse rate when supine (delta pulse rate) was significantly lower after administration of 25 mg HTL0018318 without donepezil (change of 10 bpm) compared with HTL0018318 25 mg in combination with donepezil (change of 12 bpm, mean difference of −1.6 bpm, 95% CI [−3.0; −0.2], p=0.0252). There were no significant changes in delta pulse rate after administration of 15 mg HTL0018318 or placebo without donepezil compared with the treatment in combination with donepezil. The delta pulse rate after 25 mg HTL0018318 without donepezil (change of 12 bpm) and in combination with donepezil (change of 10 bpm) was significantly lower compared with placebo without donepezil (change of 14 bpm, mean difference of −3.7 bpm, 95% CI [−6.6; −0.8], p=0.0137) and placebo in combination with donepezil (change of 15 bpm, mean difference of −3.4 bpm, 95% CI [−6.2; −0.6], p=0.0184).

Statistically significant changes were observed on saliva production, pulmonary function FEV1/FVC, LSEQ domain Quality of Sleep and LSEQ Awake following sleep (Table 8). All these changes were small and not considered to be clinically relevant.

There were no statistically significant effects on VAS alertness, calmness, mood, and nausea after HTL0018318 in combination with donepezil compared with HTL0018318 without donepezil.

All AEs were mild or moderate in intensity and there were no serious adverse events. The number of AEs reported when donepezil was administered alone did not increase after co-administering HTL0018318 15 mg and 25 mg. The percentages of subjects that reported AEs are shown in table 2. Compared with 15 mg HTL0018318 alone, co-administration of donepezil did increase the percentage of subjects reporting AEs. When 25 mg HTL0018318 was administered, a similar percentage of subjects reported AEs in the presence and absence of donepezil. The same pattern was observed in relation to percentages of subjects that reported AEs with a (possible) relation to increased cholinergic stimulation (Table 4). The most frequently reported AEs were hot flushes, hyperhidrosis, nausea, vomiting, headache and somnolence. During the study period in which 25 mg HTL0018318 or placebo was dosed together with donepezil in subjects of cohort 2, there was an outbreak of a presumed gastrointestinal viral infection at the clinical research unit. When the gastrointestinal AEs related to the viral gastroenteritis were excluded from this analysis, no vomiting was reported in any of the treatment groups, and nausea was only reported by one subject dosed with placebo in combination with donepezil and by one subject dosed with 15 mg HTL0018138 in combination with donepezil. Additionally, when excluding the viral gastroenteritis AEs, the number of AEs in the gastrointestinal disorders class was slightly higher when HTL0018318 treatment was combined with donepezil compared with HTL0018318 alone (placebo+donepezil 4 AEs vs placebo alone 1 AE; HTL0018318 15 mg+donepezil 3 AEs vs HTL0018318 15 mg alone 1 AE; and HTL0018318 25 mg+donepezil 4 AEs vs HTL0018318 25 mg alone 3 AEs).

TABLE 3
Adverse events (AEs), percentage of subjects that reported AEs. The donepezil
run in period of 20 days was followed by the combination treatment of donepezil
at steady state and HTL0018318/placebo (5 days). After a 20-day washout
period HTL0018318/placebo was administered alone (5 days).

| | Donepezil | Treatment | |
|---|---|---|---|
| | run in to | in combination | Treatment without |
| | steady state | with donepezil | donepezil |
| Placebo | | | |
| All AEs | 92% ( 12/13) | 75% ( 9/12 subjects) | 40% ( 4/10 subjects) |
| Gastrointestinal disorders | 46% ( 6/13) | 42% ( 5/12 subjects) | 10% ( 1/10 subjects) |
| Neurological disorders | 85% ( 11/13) | 33% ( 4/12 subjects) | 20% ( 2/10 subjects) |
| HTL0018318 15 mg | | | |
| All AEs | 80% ( 8/10) | 50% ( 4/8 subjects) | 43% ( 3/7 subjects) |
| Gastrointestinal disorders | 50% ( 5/10) | 13% (⅛ subjects) | 14% ( 1/7 subjects) |
| Neurological disorders | 60% ( 6/10) | 13% (⅛ subjects) | 14% ( 1/7 subjects) |
| HTL0018318 25 mg | | | |
| All AEs | 95% ( 18/19) | 88% ( 14/16 subjects) | 92% ( 11/12 subjects) |
| Gastrointestinal disorders | 53% ( 10/19) | 25% ( 4/16 subjects) | 17% ( 2/12 subjects) |
| Neurological disorders | 58% ( 11/19) | 38% ( 6/16 subjects) | 50% ( 6/12 subjects) |

TABLE 4
cholinergic AEs, percentage of subjects
that reported cholinergic AEs

| | Donepezil | Treatment in | Treatment |
|---|---|---|---|
| | run in to | combination with | without |
| | steady state | donepezil | donepezil |
| Placebo | 62% ( 8/13) | 58% | 20% |
| | | ( 7/12 subjects) | ( 2/10 subjects) |
| HTL0018318 | 60% ( 6/10) | 13% | 0% |
| 15 mg | | (⅛ subjects) | ( 0/7 subjects) |
| HTL0018318 | 63% ( 12/19) | 69% | 67% |
| 25 mg | | ( 11/16 subjects) | ( 8/12 subjects) |

### Pharmacokinetics

### HTL0018318 PK Characteristics

PK characteristics are shown in Table 5 and 6. Plasma HTL0018318 concentrations increased immediately following dosing and after Tₘₐₓ (1.74-2.5 h), plasma concentrations declined in a biphasic manner. Pharmacokinetic steady-state was reached for HTL0018318 on or before the fifth daily dose of HTL0018318.

### HTL0018318 Accumulation

The mean ratio of the AUC₀₋ₜₐᵤ of HTL0018318 after the fifth dose of HTL0018318 to AUC₀₋ₜₐᵤ after the first dose of HTL0018318 was 1.27 for 15 mg HTL0018318 and 1.23 for 25 mg HTL0018318. These ratios were comparable with donepezil co-dosed: 1.23 for 15 mg HTL0018318 and 1.21 for 25 mg HTL0018318.

The mean ratio of AUC₀₋ₜₐᵤ of HTL0018318 after fifth dose of HTL0018318 to the AUC_{0-inf} after the first dose of HTL0018318 was 1.04 following dosing with 15 mg HTL0018318 and 1.06 after 25 mg HTL0018318. These ratios were comparable with donepezil co-dosed: 1.04 for 15 mg HTL0018318 and 1.03 for 25 mg HTL0018318.

### Comparison of HTL0018318 PK Characteristics in Combination With and Without Donepezil

The ratio of the PK parameters following the first dose of HTL0018318 in combination with donepezil compared with HTL0018318 alone were 1.05 (90% CI [0.986-1.11]) for Cₘₐₓ, 1.01 (90% CI [0.793-1.28]) for Tₘₐₓ and 1.02 (90% CI [0.975-1.07]) for AUC₀₋₂₄. The ratios following the fifth dose of HTL0018318 were 1.04 for Cₘₐₓ (90% CI [0.995-1.09]), 0.974 (90% CI [0.744-1.28]) for Tₘₐₓ, 1.00 (90% CI [0.969-1.03]) for AUC₀₋ₜₐᵤ and 0.911 (90% CI [0.854-0.972]) for Cₘᵢₙ.

TABLE 5
Group summary data of HTL0018318 plasma and urine pharmacokinetic parameters following
the first and fifth dose of 15 mg HTL0018318 with and without donepezil co dosing.

| | | | | | Half- | | | | | |
|---|---|---|---|---|---|---|---|---|---|---|
| | | | Tmax* | Cₘₐₓ | life | AUC_{0-inf} | AUC₀₋ₜₐᵤ | CL/F | Ae | CLr |
| | Dosage | | (h) | (ng/mL) | (h) | (ng · h/mL) | (ng · h/mL) | (L/h) | (%) | (L/h) |
| 1^{st} | 15 mg | N | 7 | 7 | 7 | 7 | 7 | 7 | 7 | 7 |
| dose | only | Mean | 2.00 | 83.4 | 9.25 | 1130 | 919 | 14.2 | 39.2 | 6.55 |
| | | SD | (1.00-4.00) | 9.78 | 1.81 | 307 | 198 | 4.29 | 7.97 | 1.31 |
| | | CV % | | 11.7 | 19.5 | 27.2 | 21.6 | 30.2 | 20.3 | 20.0 |
| 5^{th} | 15 mg | N | 7 | 7 | 7 | | 7 | 7 | 7 | 7 |
| dose | only | Mean | 2.05 | 103 | 13.8 | | 1180 | 14.0 | 47.9 | 6.24 |
| | | SD | (1.00-4.00) | 25.2 | 1.47 | | 380 | 4.92 | 13.1 | 0.974 |
| | | CV % | | 24.5 | 10.7 | | 32.2 | 35.1 | 27.3 | 15.6 |
| 1^{st} | 15 mg + | N | 8 | 8 | 8 | 8 | | 8 | 8 | 8 |
| dose | DPZ | Mean | 2.50 | 91.2 | 8.97 | 1120 | 932 | 14.7 | 39.9 | 6.53 |
| | | SD | (1.00-4.00) | 25.6 | 2.24 | 335 | 243 | 4.83 | 11.8 | 1.35 |
| | | CV % | | 28.1 | 25.0 | 30.0 | 26.0 | 32.9 | 29.6 | 20.7 |
| 5^{th} | 15 mg + | N | 8 | 8 | 8 | | 8 | 8 | 7 | 7 |
| dose | DPZ | Mean | 1.74 | 109 | 11.6 | | 1150 | 14.0 | 51.5 | 6.54 |
| | | SD | (0.50-4.00) | 23.2 | 1.43 | | 324 | 4.12 | 14.2 | 1.51 |
| | | CV % | | 21.3 | 12.3 | | 28.2 | 29.4 | 27.6 | 23.1 |

Arithmetic mean ± SD, % CV except * median (min-max).
Tau = 24 hrs.

TABLE 6
Group summary data of HTL0018318 plasma and urine pharmacokinetic parameters following
the first and fifth dose of 25 mg HTL0018318 with and without donepezil co dosing.

| | | | | | Half- | | | | | |
|---|---|---|---|---|---|---|---|---|---|---|
| | | | Tmax* | Cₘₐₓ | life | AUC_{0-inf} | AUC₀₋ₜₐᵤ | CL/F | Ae | CLr |
| | Dosage | | (h) | (ng/mL) | (h) | (ng · h/mL) | (ng · h/mL) | (L/h) | (%) | (L/h) |
| 1^{st} | 25 mg | N | 12 | 12 | 12 | 12 | 12 | 12 | 12 | 12 |
| dose | only | Mean | 1.75 | 135 | 8.52 | 1650 | 1370 | 17.6 | 32.6 | 6.21 |
| | | SD | (0.650-3.00) | 27.2 | 2.50 | 748 | 468 | 6.17 | 10.5 | 1.89 |
| | | CV % | | 20.1 | 29.3 | 45.3 | 34.2 | 35.1 | 32.2 | 30.4 |
| 5^{th} | 25 mg | N | 12 | 12 | 12 | | 12 | 12 | 11 | 11 |
| dose | only | Mean | 1.76 | 162 | 13.4 | | 1710 | 16.6 | 44.1 | 6.56 |
| | | SD | (1.50-3.00) | 41.8 | 1.83 | | 683 | 5.98 | 14.9 | 1.38 |
| | | CV % | | 25.8 | 13.7 | | 39.9 | 36.0 | 33.8 | 21.0 |
| 1^{st} | 25 mg + | N | 16 | 16 | 16 | 16 | 16 | 16 | 16 | 16 |
| dose | DPZ | Mean | 1.76 | 139 | 8.73 | 1710 | 1410 | 17.0 | 41.1 | 7.76 |
| | | SD | (0.267-3.02) | 29.5 | 2.52 | 793 | 482 | 6.05 | 13.3 | 2.60 |
| | | CV % | | 21.2 | 28.9 | 46.4 | 34.2 | 35.6 | 32.4 | 33.5 |
| 5^{th} | 25 mg + | N | 13 | 13 | 13 | | 13 | 13 | 8 | 8 |
| dose | DPZ | Mean | 2.00 | 171 | 10.7 | | 1740 | 16.0 | 47.7 | 6.78 |
| | | SD | (1.50-4.00) | 45.7 | 2.14 | | 664 | 5.15 | 8.48 | 1.95 |
| | | CV % | | 26.7 | 20.0 | | 38.2 | 32.2 | 17.8 | 28.8 |

Arithmetic mean ± SD, % CV except * median (min-max).
Tau = 24 hrs.

### Donepezil

The mean plasma donepezil concentration immediately before the first administration of 15 mg HTL0018318 (15 hours post donepezil dose) was 40.5 ng/ml (CV 25.0%), before 25 mg HTL0018318 was 37.4 ng/ml (CV 28.8%) and before placebo was 36.1 ng/ml (CV 29.6%). Plasma donepezil concentrations after the 18^{th} to 24^{th} doses suggested that donepezil was at pharmacokinetic steady-state by the time of the 18^{th} donepezil dose. The geometric mean ratios of the donepezil concentration at 4, 15 or 24 hours post-dosing with HTL0018318 at 15 or 25 mg on the first dose of HTL0018318 or at steady-state versus donepezil plasma concentrations immediately before co-dosing (18^{th} donepezil dose) was between 0.961 and 1.06 with the 90% CI including unity for all comparisons. The corresponding donepezil concentrations associated with dosing HTL0018318 placebo fell in the range 0.915 to 1.06 with the 90% CI including unity except at 24 hours post dose on Day 1 of placebo administration where the ratio was 0.915 (90% CI 0.871-0.962).

### Discussion

This randomized, double-blind, placebo-controlled trial in 42 (to deliver 36) healthy elderly subjects investigated safety and tolerability and PK of repeated doses of HTL0018318 (15 mg or 25 mg) given without and in combination with donepezil (10 mg) at steady state. An effect on tolerability could have been predicted since both donepezil and HTL0018318 enhance cholinergic activity. There was no a priori expectation of a pharmacokinetic drug-drug interaction.

AEs were reported by a high proportion of the subjects during the donepezil run-in phase. Multiple doses of HTL0018318 in combination with donepezil were generally well tolerated. When 15 mg HTL0018318 and placebo were combined with donepezil, a greater proportion of subjects reported AEs compared with HTL0018318 or placebo alone. This difference is likely caused by donepezil, as donepezil alone resulted in more AEs than HTL0018318 without donepezil. Since 25 mg HTL0018318 without donepezil led to a comparable percentage of subjects experiencing AEs as donepezil alone, there was no difference when the treatments were combined.

The side effect profile observed in this study is comparable to that observed in the single ascending dose (SAD) and multiple ascending dose (MAD) studies with HTL0018318 ((Bakker C, Tasker T, Liptrot J, Hart E P, Klaassen E. S, Doll R. J, et al. Alzheimer's Research & Therapy. 2020; (submitted); Bakker C, Tasker T, Liptrot J, Hart E P, Klaassen E S, Prins S, et al. Br J Clin Pharmacol. 2020; (submitted)). Only nausea and vomiting were reported more frequently than in the SAD and MAD study. During the study period in which 25 mg HTL0018318 or placebo were dosed in combination with donepezil in subjects of cohort 2, there was an outbreak of a presumed gastrointestinal viral infection at the clinical research unit. The presumption of viral gastroenteritis was based on the clinical presentation of the symptoms and the fact that staff of the clinical research organisation and placebo subjects were affected as well. Additionally, the onset of the symptoms of each individual followed one after the other and was not related to the timing of dosing.

The statistically significant increases in supine systolic blood pressure after administration of 25 mg HTL0018318 in combination with donepezil (1.6 mm Hg) and after placebo in combination with donepezil (1.7 mm Hg) are considered to be of small magnitude and not of clinical concern. The pulse rate data suggest that the combination of HTL0018318 and donepezil may decrease supine pulse rate, but not standing pulse rate compared with HTL0018318 without donepezil. Accordingly, the physiological heart rate increment after standing up was greater in those who had received HTL0018318 in combination with donepezil versus HTL0018318 without donepezil. However, these changes were similarly of small magnitude (up to 1.6 bpm) and of no clinical concern.

Increased saliva production was expected based on the mechanism of action of HTL0018318 (Bymaster F P, Carter P A, Yamada M, Gomeza J, Wess J, Hamilton S E, et al. The European journal of neuroscience. 2003; 17(7):1403-10), and because salivary hypersecretion has been described in other studies investigating M₁ mAChR agonists (Nathan P J, Watson J, Lund J, Davies C H, Peters G, Dodds C M, et al. Int J Neuropsychopharmacol. 2013; 16(4):721-31; Voss T, Li J, Cummings J, Farlow M, Assaid C, Froman S, et al. Alzheimer's & dementia (New York, N Y). 2018; 4:173-81; Sramek J J, Hurley D J, Wardle T S, Satterwhite J H, Hourani J, Dies F, et al. J Clin Pharmacol. 1995; 35(8):800-6), whereas it is not a common side effect of donepezil (Birks J S, Harvey R J. Donepezil for dementia due to Alzheimer's disease. The Cochrane database of systematic reviews. 2018; 6:Cd001190). In the current study, the small changes on production of saliva are not considered of clinical importance (Table 8).

Acetylcholine can elicit bronchoconstriction and mucous secretion by activating the M₂ and M₃ mAChRs on the airway smooth muscle and mucous glands. The M₁ mAChRs might play a minor role as agonism of the M₁ mAChRs at the postganglionic nerves facilitates acetylcholine release in the synaptic junction. This stimulates the M₃ mAChRs which contributes to bronchoconstriction and mucous secretion (Buels K S, Fryer A D. Muscarinic receptor antagonists: effects on pulmonary function. Handbook of experimental pharmacology. 2012(208):317-41; Castro JMdA, Resende R R, Mirotti L, Florsheim E, Albuquerque L L, Lino-dos-Santos-Franco A, et al. BioMed Research International. 2013; 2013:805627). The observed increase of FEV1/FVC in the current study suggesting less constriction is therefore not considered to be a pharmacological effect and not clinically important.

The M₁ and M₃ mAChRs play an essential role in the rapid eye movement phase during the sleep wake cycle (Niwa Y, Kanda G N, Yamada R G, Shi S, Sunagawa G A, Ukai-Tadenuma M, et al. Cell reports. 2018; 24(9):2231-47.e7). In the current study, no clinically relevant changes were observed on the LSEQ after administration of HTL0018318 alone or HTL0018318 in combination with donepezil (Table 8).

The pharmacokinetics of HTL0018318 were well-characterized in plasma and urine. The characteristics were comparable to the PK data observed in previous studies (Bakker C, Tasker T, Liptrot J, Hart E P, Klaassen E. S, Doll R. J, et al. Alzheimer's Research & Therapy. 2020; (submitted); Bakker C, Tasker T, Liptrot J, Hart E P, Klaassen E S, Prins S, et al. Br J Clin Pharmacol. 2020; (submitted)). Mean Tₘₐₓ (1.74-2.5 h) and half-life following the fifth dose (10.7-13.8 h) did not appear to change with respect to HTL0018318 dose level and co-dosing with donepezil. There was no apparent change in renal elimination of HTL0018318 due to changing HTL0018318 dose level or due to co-dosing with donepezil. Variability of the HTL0018318 plasma PK Cₘₐₓ, AUC₀₋ₜₐᵤ and apparent elimination half-life was similar between the 15 mg and 25 mg dose groups and similar between the periods with and without donepezil co-dosing (between 11.7% and 39.9%). There appeared to be no trend in degree of accumulation related to HTL0018318 dose level or related to co-dosing with donepezil. Comparisons of the ratios for Cₘₐₓ, Tₘₐₓ, AUC₀₋₂₄, AUC₀₋ₜₐᵤ and Cₘᵢₙ (between 0.911 and 1.05) of HTL0018318 measured during the HTL0018318 dosing period with and without co-administration of donepezil indicate that donepezil does not have a meaningful impact on the PK of HTL0018318.

The plasma donepezil concentrations before the first administration of HTL0018318/placebo were considered to be therapeutic (Ota T, Shinotoh H, Fukushi K, Kikuchi T, Sato K, Tanaka N, et al. Clinical neuropharmacology. 2010; 33(2):74-8; Shiraishi T, Kikuchi T, Fukushi K, Shinotoh H, Nagatsuka S, Tanaka N, et al. Neuropsychopharmacology. 2005; 30(12):2154-61). Comparisons of the plasma donepezil concentrations measured with and without co-administration HTL0018318 indicate that HTL0018318 does not impact the PK of donepezil (mean ratios between 0.915 and 1.06). Overall, HTL0018318 given in combination with donepezil to elderly healthy subjects was generally well tolerated, did not lead to clinical, safety or PK concerns and would be a viable combination treatment, at these dose levels, for the treatment of patients with Alzheimer's disease.

TABLE 7
Visit and Assessment Schedule

| | | | DPZ | | DPZ | Steady |
|---|---|---|---|---|---|---|
| | Screening | Pre- | 5 mg | DPZ | 10 mg | State |
| Period 1: Donepezil | Day −28 | DPZ | Day 1 | 10 mg | Day 6 | DPZ |
| run-in period | to −2 | Day 0 | to 4 | Day 5 | to 18 | Day 19 |
| Admission | X | X | | X | X¹ | |
| Informed consent | X | | | | | |
| In- & exclusion criteria, Demography | X | | | | | |
| Medical- and con-med. history | X | | | | | |
| Virology | X | | | | | |
| Cotinine test | X | | | | | |
| Physical examination, Temperature | X | X | | | | X |
| BsHaem, BsChem, Urinalysis | X | X² | | | | |
| Urine Drug, Pregnancy³, Breath Alc. | X | X | | X | X | |
| Height⁴ and weight | X | X | | | | |
| ECG 12 lead & Vital Signs (HR, BP) | X | X | | X | | X |
| ECG 24 h Holter | X | | | | | X |
| Donepezil administration | | X⁵ | X⁵ | X⁵ | X⁶ | X |
| Salivary flow rate | | X | | | | X |
| Respiratory function | | X⁷ | | | | X |
| VAS Bond & Lader, nausea, LSEQ | X | X | | X | | X |
| Blood PK sample donepezil | | | | X | X⁸ | X⁸ |

| | |
|---|---|
| iPod rating of nausea and sleep quality | □ X □ |

| | | | |
|---|---|---|---|
| (S)AE/Con-med monitoring and compliance, | X⁹ | X¹⁰ | □ X □ |
| General symptoms | | | |

DPZ = Donepezil, Con-med = Concomitant Medications, Br(eath). Alc = Breath alcohol test, Vital signs = heart rate and blood
pressure, PK = Pharmacokinetics, (S)AE = (Serious) Adverse Event, BsHaem = Blood Sample Haematology, BsChem = Blood
Sample Chemistry, ECG = Electrocardiogram, LSEQ = Leeds Sleep Evaluation Questionnaire, VAS = visual analog scale.
AE = Adverse Event
Period 2 (HTL0018318/placebo in combination with donepezil)
and Period 3 (HTL0018318/placebo alone)

| | | | | | | | | | |
|---|---|---|---|---|---|---|---|---|---|
| Pre-dose | Day 20 | Day 21 | Day 22 | Day 23 | Day 24 | Day 25 | Day 26 | Day 27 | Follow-Up |

¹Stay overnight at the clinical research uni from the evening of Day 18 until the morning of Day 25.
²Includes pharmacogenetic sample
³Serum pregnancy test at screening, urine pregnancy test on other visits.
⁴Height measured only at screening.
⁵Donepezil 5 mg treatment (5 days) dispensed with instructions. Self-administration each evening before retiring.
⁶Donepezil 10 mg treatment (13 days) dispensed with instructions. Self-administration each evening before retiring.
Administration at clinical research unit on Day 18
⁷Performed twice
⁸Donepezil PK samples on Day 18 pre-dose, 4 h post dose, on Day 19 at 15 h and 24 h post Day 18 dose (=pre-Day 19 dose),
and 4 h post Day 19 dose.
⁹Baseline conditions at screening until pre-dose Day 0; continuous monitoring from Day 0.
¹⁰A clinical research unit physician will review subject eligibility before dosing
Period 2 (HTL0018318/placebo in combination with donepezil)
and Period 3 (HTL0018318/placebo alone)

| | Pre-dose | | | | | | | | | |
|---|---|---|---|---|---|---|---|---|---|---|
| | Day | Day 20 | Day 21 | Day 22 | Day 23 | Day 24 | Day 25 | Day 26 | Day 27 | Follow-Up |
| | 20 & 45 | Day 45 | Day 46 | Day 47 | Day 48 | Day 49 | Day 50 | Day 51 | Day 52 | Day 56-63 |
| Stay overnight at CHDR | X¹¹ | X | X | X | X | X | | | | |
| Weight | X | | | | | | X | | | X |
| Physical Examination | | | | | | | | | | |
| BsHaem, BsChem, Urinalysis | X | | | X¹² | | | X¹² | | | X |
| Temperature | X | | X¹² | X² | X² | X² | X | | | X |
| Urine drug, pregnancy, Br. alc. | X¹³ | | | | | | | | | |
| ECG 12 lead & Vital Signs | X | X⁴ | X¹⁴ | X⁴ | X⁴ | X⁴ | X | | | X |
| ECG 24 h Holter | | X | | X | | X | | | | |
| HTL0018318/placebo | | X | X | X | X | X | | | | |
| Administration Donepezil | | X¹⁵ | X⁵ | X⁵ | X⁵ | | | | | |
| Salivary flow rate | X | X¹⁶ | | X⁶ | | X⁶ | | | | |
| Respiratory function ¹⁷ | X | X | | X | | X | X | | | |
| VAS Bond & Lader, nausea ¹⁸ | X | X | X | X | X | X | X | | | |
| LSEQ | X | | X² | X² | X² | X² | X | | | |
| Blood PK donepezil ¹⁹ | X | X | X | X | X | X | X | X | X | X |
| Blood PK HTL0018318²⁰ | X | X | X | X | X | X | X | X | X | X |
| Urine PK collection ²¹ | X | X | X | X | X | X | X | X | X | X |

| | |
|---|---|
| (S)AE/Con-meds monitoring, | □ X □ |
| general symptoms | |

¹¹Stay overnight on Day 19 and Day 44
¹²Pre-dose (all ‘pre-dose’ references concern HTL0018318)
¹³Day 44 only
¹⁴Pre-dose, 0.5 h, 1 h, 2 h, 3 h and 6 h post dose
¹⁵Donepezil 10 mg administered on Days 20 to 23 in the evening
¹⁶Before breakfast, 2 h, 4 h, 6 h post dose (pre-meals)
¹⁷ Pre-dose, 2 h and 6 h post dose on Days, 20/45, 22/47 and 24/49
¹⁸ Pre-dose, 2 h, 4 h, 8 h post dose on Days 20/45; Pre-dose, 2 h and 8 h post dose on Days 21 to 24 and 46 to 49.
¹⁹ Donepezil PK: Day 20 at 15 h post (Day 19) dose, pre-dose Day 20, 4 h post dose. Day 21, 22, 23: 15 h
post previous day's dose. Day 23 pre-dose, 4 h, 15 h, 27 h, 39 h, 63 h, 87 h, 7-14 days post dose (14 samples in total).
²⁰HTL0018318 PK: Day 20/45 pre-dose. Day 20/45 0.25 h, 0.5 h, 1 h, 1.5 h, 2 h, 3 h, 4 h, 8 h, 9 h (Day 20 only),
12 h, 13 h (Day 20 only) post-dose. Day 21, 22, 23/46, 47, 48 pre-dose. Day 24/49 pre-dose, 0.25 h, 0.5 h, 1 h, 1.5 h, 2 h, 3 h,
4 h, 8 h, 12 h, 24 h, 48 h, 72 h, 7-14 days post-dose.
²¹ Day 20/45 pre-dose (spot). Day 20/45 and 24/49: 0-4 h (complete [c]), 4-8 h (c), 8-12 h (c), 12-24 h (c).
Day 25/50 (24-48 h) (c). Day 26/51 (48-72 h) (c). Follow-up (spot)

TABLE 8
Results of the statistical analysis of safety and tolerability measurements

| | | Contrasts | | | | | | |
|---|---|---|---|---|---|---|---|---|
| | | | | | | | 15 mg | 25 mg |
| | | 15 mg | 25 mg | | | | HTL0018318 | HTL0018318 |
| | | HTL0018318 + | HTL0018318 + | 15 mg | 25 mg | Placebo | alone vs | alone vs |
| | | donepezil | donepezil | HTL0018318 | HTL0018318 | alone vs | 15 mg | 25 mg |
| | Treatment | vs | vs | alone vs | alone vs | placebo + | HTL0018318 | HTL0018318 |
| Parameter | P-value | Placebo | Placebo | Placebo | Placebo | donepezil | donepezil | donepezil |
| Saliva (g) | 0.0930 | 0.707 | 0.910 | 0.498 | 0.906 | −0.345 | −0.554 | −0.349 |
| | | (−0.280, | (0.066, | (−0.503, | (0.057, | (−0.703, | (−0.982, | (−0.712, |
| | | 1.694) | 1.754) | 1.499) | 1.756) | 0.013) | −0.125) | 0.015) |
| | | p = 0.1532 | p = 0.0355* | p = 0.3169 | p = 0.0373* | p = 0.0579 | p = 0.0144* | p = 0.0591 |
| FEV 1 | 0.1341 | 0.043 | 0.085 | 0.153 | 0.077 | 0.016 | 0.127 | 0.008 |
| second (L) | | (−0.089, | (−0.030, | (0.012, | (−0.047, | (−0.096, | (−0.008, | (−0.095, |
| | | 0.175) | 0.201) | 0.295) | 0.200) | 0.129) | 0.262) | 0.110) |
| | | p = 0.5204 | p = 0.1455 | p = 0.0340* | p = 0.2197 | p = 0.7705 | p = 0.0642 | p = 0.8785 |
| FVC (L) | 0.2037 | 0.03719 | 0.17837 | 0.20348 | 0.06192 | 0.00389 | 0.17019 | −.11255 |
| | | (−.18178, | (−.01162, | (−.03215, | (−.14530, | (−.20241, | (−.07662, | (−.29932, |
| | | 0.25616) | 0.36836) | 0.43911) | 0.26915) | 0.21019) | 0.41699) | 0.07421) |
| | | p = 0.7350 | p = 0.0652 | p = 0.0891 | p = 0.5518 | p = 0.9695 | p = 0.1688 | p = 0.2275 |
| FEV1/FVC | 0.7598 | 1.052 | −0.399 | −0.327 | 1.600 | 0.492 | −0.886 | 2.491 |
| (%) | | (−1.237, | (−2.388, | (−2.754, | (−0.537, | (−1.481, | (−3.201, | (0.727, |
| | | 3.341) | 1.590) | 2.101) | 3.736) | 2.466) | 1.429) | 4.255) |
| | | p = 0.3606 | p = 0.6894 | p = 0.7883 | p = 0.1389 | p = 0.6108 | p = 0.4359 | p = 0.0078** |
| PEF | 0.0856 | 0.339 | 0.273 | 0.282 | 0.606 | −0.286 | −0.343 | 0.047 |
| (L/sec) | | (−0.280, | (−0.265, | (−0.377, | (0.033, | (−0.868, | (−1.036, | (−0.481, |
| | | 0.958) | 0.811) | 0.942) | 1.179) | 0.296) | 0.350) | 0.575) |
| | | p = 0.2771 | p = 0.3144 | p = 0.3947 | p = 0.0385* | p = 0.3291 | p = 0.3253 | p = 0.8591 |
| VAS | 0.1296 | −0.70 | −1.49 | −1.96 | −1.63 | 0.80 | −0.46 | 0.66 |
| Alertness | | (−2.71, | (−3.22, | (−4.04, | (−3.43, | (−0.48, | (−1.91, | (−0.43, |
| (mm) | | 1.30) | 0.25) | 0.12) | 0.18) | 2.08) | 0.99) | 1.76) |
| | | p = 0.4843 | p = 0.0908 | p = 0.0637 | p = 0.0759 | p = 0.2086 | p = 0.5217 | p = 0.2221 |
| VAS | 0.1369 | −2.37 | −1.67 | −3.17 | −2.34 | 0.59 | −0.20 | −0.07 |
| Calmness | | (−5.39, | (−4.23, | (−6.21, | (−4.92, | (−0.20, | (−1.08, | (−0.75, |
| (mm) | | 0.65) | 0.89) | −0.13) | 0.25) | 1.38) | 0.68) | 0.60) |
| | | p = 0.1193 | p = 0.1940 | p = 0.0418* | p = 0.0748 | p = 0.1406 | p = 0.6535 | p = 0.8264 |
| VAS Mood | 0.0908 | −0.24 | −0.38 | −2.93 | −2.49 | 2.66 | −0.03 | 0.55 |
| (mm) | | (−2.38, | (−2.22, | (−5.23, | (−4.49, | (0.54, | (−2.55, | (−1.32, |
| | | 1.90) | 1.45) | −0.63) | −0.48) | 4.77) | 2.49) | 2.43) |
| | | p = 0.8214 | p = 0.6763 | p = 0.0135* | p = 0.0158* | p = 0.0167* | p = 0.9796 | p = 0.5414 |
| VAS | 0.5595 | −0.08 | −0.04 | −0.06 | −0.05 | −0.02 | −0.01 | −0.03 |
| Nausea | | (−0.23, | (−0.16, | (−0.21, | (−0.17, | (−0.06, | (−0.05, | (−0.07, |
| (log(mm)) | | 0.06) | 0.08) | 0.08) | 0.07) | 0.01) | 0.04) | 0.00) |
| | | p = 0.2569 | p = 0.4963 | p = 0.3780 | p = 0.4239 | p = 0.2017 | p = 0.7832 | p = 0.0683 |
| LSEQ: | 0.5617 | 1.59 | −0.74 | 0.82 | 1.01 | −0.02 | −0.79 | 1.72 |
| Getting to | | (−1.37, | (−3.31, | (−2.31, | (−1.71, | (−2.48, | (−3.71, | (−0.51, |
| sleep | | 4.55) | 1.82) | 3.96) | 3.73) | 2.43) | 2.12) | 3.95) |
| | | p = 0.2860 | p = 0.5645 | p = 0.6005 | p = 0.4619 | p = 0.9838 | p = 0.5818 | p = 0.1246 |
| LSEQ: | 0.7988 | 2.42 | −1.37 | −5.14 | −2.18 | 7.30 | −0.26 | 6.49 |
| Quality of | | (−5.47, | (−8.02, | (−13.38, | (−9.34, | (0.79, | (−8.18, | (0.57, |
| sleep | | 10.31) | 5.27) | 3.10) | 4.97) | 13.81) | 7.66) | 12.41) |
| | | p = 0.5408 | p = 0.6804 | p = 0.2168 | p = 0.5430 | p = 0.0293* | p = 0.9473 | p = 0.0328* |
| LSEQ: | 0.3958 | 3.50 | 0.52 | −1.72 | −1.85 | 3.29 | −1.93 | 0.93 |
| Awake | | (0.19, | (−2.39, | (−5.17, | (−4.85, | (0.17, | (−5.55, | (−1.86, |
| following | | 6.81) | 3.42) | 1.74) | 1.15) | 6.41) | 1.70) | 3.71) |
| sleep | | p = 0.0385* | p = 0.7232 | p = 0.3222 | p = 0.2213 | p = 0.0389* | p = 0.2909 | p = 0.5073 |
| LSEQ: | 0.8954 | 1.51 | −0.80 | −1.00 | −0.60 | 1.66 | −0.85 | 1.86 |
| Behaviour | | (−3.87, | (−5.54, | (−6.57, | (−5.46, | (−2.12, | (−5.19, | (−1.50, |
| after wake | | 6.90) | 3.95) | 4.56) | 4.26) | 5.45) | 3.48) | 5.22) |
| | | p = 0.5738 | p = 0.7367 | p = 0.7185 | p = 0.8036 | p = 0.3755 | p = 0.6887 | p = 0.2670 |

### Example D

### Study Design

This was a randomised, double-blind, parallel-group, placebo-controlled study in which patients with mild-to-moderate AD received placebo or one of three doses of HTL0018318 over a 4-week period as an adjunctive treatment to standard of care donepezil (10 mg per day) with or without memantine. The study was an outpatient study conducted across 18 centres in Poland, Czech Republic, Spain, and Slovakia (NCT03456349, registered at clinicaltrials.gov). Patients were assessed for eligibility during a 42-day screening period and randomised equally to either placebo or a HTL0018318 target dose of 5 mg, 15 mg, or 25 mg per day (FIG. 5A). HTL0018318 was titrated to the target dose during a 2-week titration period (FIG. 5B); investigators could maintain or reduce the dose to limit tolerability/safety issues throughout this period. The study was conducted in accordance with the principles of the Declaration of Helsinki, the International Council for Harmonisation good clinical practice guidelines, and according to all appropriate country regulatory requirements. Relevant independent ethics committees provided approval, and all patients (or their caregiver) provided written informed consent.

### Patients

Eligible patients were aged 55-85 years, with diagnostic evidence of probable AD according to the 2011 National Institute of Aging-Alzheimer's Association criteria, (McKhann G M, Knopman D S, Chertkow H, Hyman B T, Jack C R, Jr., Kawas C H, et al. Alzheimers Dement. 2011; 7:263-9) mild-to-moderate dementia according to a mini-mental state examination (MMSE) score of 12-24, and taking donepezil at a stable dose of 10 mg daily (with or without memantine), for at least 6 weeks prior to screening. Full inclusion and exclusion criteria are reported below:

### Study Inclusion Criteria

1. Aged 55-85 years, except in Czech Republic where the upper age limit for participation was 75 years.
2. Women of non-childbearing potential: defined as either surgically sterile (bilateral tubal ligation, both ovaries removed, or hysterectomy) or post-menopausal (aged >60 years, or 55-60 years and amenorrheic for years with a post-menopausal follicle-stimulating hormone level).
3. Men who were surgically sterile, or if sexually active, whose partners agreed to use effective contraception (unless they were post-menopausal) from screening to 28 days after dosing.
4. Evidence of probable Alzheimer's disease (AD) according to the 2011 National Institute on Aging—Alzheimer's Association criteria at screening.
5. Treatment with donepezil 10 mg once daily for weeks prior to screening (co-administration with a stable dose of memantine was allowed, except in Poland).
6. A diagnosis of probable AD confirmed by magnetic resonance imaging (MRI) or computed tomography (CT; if MRI was not feasible) within the last 12 months without any other clinically significant comorbid pathologies. Diagnoses with evidence of cortical infarct, strategically located subcortical grey matter infarct (eg, hippocampus, thalamus), multiple white matter lacunas, or extensive white matter abnormalities, were excluded. Where the MRI or CT scan was performed 12-24 months prior to screening, or where there was evidence suggestive of stroke or other possible neurological disease with onset between the time of the last MRI or CT scan and the screening evaluation, an MRI or CT scan was repeated at screening.
7. Mild to moderate dementia with a mini-mental state examination score 12-24 at screening.
8. Modified Rosen Hachinski Ischemia score
9. Lived in the community or in a community dwelling, with a reliable caregiver(s) or informant who agreed to accompany the patient to all clinic visits, provide information about the patient as required by the protocol, and oversee daily medication adherence.
10. Ability to swallow oral medication in the form of capsules.
11. General good health based on medical history, physical examination, vital signs, and 12-lead electrocardiogram (ECG), with no serious or unstable disease 3 months prior. Mild, chronic, stable disease (eg, controlled osteoarthritis) was allowed at the investigator's discretion.
12. Normal sinus rhythm without any clinically significant ECG conduction abnormalities as assessed by the investigator using a screening 12-lead ECG.
13. Patients with type 2 diabetes stabilised by diet and/or oral hypoglycaemic agents (3 months on a stable dose) were eligible provided they were medically managed by their treating physician and monitored regularly to ensure adequacy of control. Patients must have had a haemoglobin A1c of <7.7% and a random serum glucose value of 170 mg/dL at screening. Insulin use was not allowed.
14. Previously able to read, write, and communicate effectively with others in their native language in pre-dementia state.
15. Able to participate in and complete study assessments including electroencephalography (EEG; adequate hearing is required to undertake the EEG assessment).
16. Able to provide written informed consent (patient [if possible] and caregiver).

### Study Exclusion Criteria

1. Treatment with cholinesterase inhibitors other than donepezil.
2. Treatment with memantine monotherapy.
3. Treatment with anti-cholinergic and/or anti-muscarinic agents, including overactive bladder treatments, antihistamines, antipsychotics, and tricyclic antidepressants, within 12 weeks before screening.
4. Use of strong inhibitors of cytochrome P450 (CYP) 2D6 (eg, quinidine, paroxetine, fluoxetine, terbinafine, bupropion) or strong or moderate inhibitors/inducers of CYP3A4 or CYP2C9 (eg, ketoconazole, rifampicin, fluconazole, carbamazepine), or herbal preparations containing St. John's wort (Hypericum perforatum) within 21 days before the baseline visit.
5. Use of strong inducers (eg, phenytoin) or strong inhibitors (eg, ciprofloxacin, fluvoxamine, valproate, clarithromycin) of any CP450 within 21 days before the baseline visit.
6. Use of other drugs that could affect cognition (eg, benzodiazepines or γ-Aminobutyric acid type A [GABA-A] receptor agonists used as anxiolytics, sedative hypnotics) or over-the-counter sleeping aids within 12 weeks before screening.
7. Use of hormone replacement therapy, thyroid supplement, vitamin E, or vitamin B12, unless at a stable dose, 4 weeks before the baseline visit.
8. Severe behavioural disturbances at screening (eg, extreme agitation, physical aggression or violence) that could compromise assessment of cognition.
9. Comorbidities that could have contributed to cognitive dysfunction, including but not limited to: other neurodegenerative disorders (eg, Lewy body dementia, fronto-temporal dementia, vascular dementia); psychiatric disorders that meet the Diagnostic and Statistical Manual of Mental Disorders criteria for psychotic disorders, depression, bipolar disorder, epilepsy, or Parkinson's disease; a known history of alcohol or substance abuse (within the past year); acquired immune deficiency of alcohol and syndrome dementia; tertiary syphilis; or any non-AD dementia.
10. A score on the 15-item Geriatric Depression Scale.
11. Evidence of clinically significant suicidal ideation within 6 months prior to screening, as defined by suicidal ideation or active plan according to items 4 or 5 of the Columbia Suicide Severity Rating Scale (C-SSRS). Patients with a prior suicide attempt of any sort, or prior serious suicidal ideation or plan within the last 10 years, were enrolled after suicidal ideation was excluded per investigator assessment.
12. Thyroid disease, unless the patient was euthyroid and stable on treatment for weeks before screening. Patients were required to maintain stable treatment throughout the study.
13. A personal or family history of congenital long QT syndrome or sudden death. Baseline Fridericia correction (QTcF) should have been >450 ms for men and >470 ms for women at screening or any other clinically significant abnormality on ECG at screening (may have been repeated once).
14. Clinically significant cardiovascular disease within 6 months prior to screening. Examples include myocardial infarction; coronary artery bypass graft; percutaneous transluminal coronary angioplasty; severe or unstable angina; a serious arrhythmia; clinically significant ECG conduction abnormalities (eg, sinus bradycardia, sinoatrial block or atrioventricular junction block, left or right bundle branch block); or moderate-to-severe heart failure.
15. History of clinically significant orthostatic blood pressure (BP) changes or orthostatic symptoms (eg, dizziness, light-headedness), hypotension, or bradycardia. Patients with mild and well-controlled hypertension were eligible (ie, with a resting BP of 150/90 (140/90 in Czech Republic) and on different anti-hypertensive agents for BP management).
16. Signs and symptoms of peripheral vascular disease such as claudication; peripheral oedema; skin changes; cramping; thromboembolic disease; atherosclerotic disease of the iliac; femoral, popliteal, and carotid arteries; and aneurysms of the femoral, iliac, and popliteal arteries (but more specifically abdominal and thoracic aneurysms). Patients with connective tissue disorders such as Marfan's Syndrome and scleroderma or other causes of peripheral vascular disease. Any history within 6 months of screening, of patient undergoing resections or repairs or bypasses of any of these peripheral arteries was also excluded.
17. Transient ischemic attack in the past 6 months.
18. Any history of cerebrovascular accident or stroke.
19. Any history of a seizure disorder other than a single febrile seizure.
20. Pulmonary disease (eg, interstitial lung disease, moderate or severe or unstable asthma, moderate or severe chronic obstructive pulmonary disease, moderate or severe emphysema or chronic bronchitis, or chronic or acute pneumonitis) or evidence of clinically significant moderate or severe pulmonary symptoms. Mild asthma not requiring treatment for year was allowed.
21. History of cancer within the last 5 years. Treated basal cell or squamous cell carcinoma of the skin was allowed, and stable, localised prostate cancer not requiring treatment was allowed.
22. Evidence or history of diabetes mellitus type 1. Treatment with insulin.
23. Clinically significant laboratory abnormalities at screening.
24. Serum creatinine >1.5× upper limit of normal (ULN).
25. Clinically significant liver blood tests increase of >2×ULN.
26. Presence of significant adverse events (AEs) typical for donepezil treatment (eg, diarrhoea, nausea, vomiting) or peptic ulcers in medical history. Patients with low incidence of AEs with donepezil were allowed at the investigator's discretion.
27. Diagnosed B12 deficiency (patients who took B12 as an intermittent supplement were permitted) or current B12 below the laboratory reference normal range and an elevated methylmalonic acid.
28. Inability to complete the behavioural or EEG assessments of the study due to significant sensory (impaired hearing or vision) or movement difficulties.
29. Any condition possibly affecting drug absorption (eg, gastrectomy).
30. Body weight <40 kg (Czech Republic only).
31. Any other concurrent medical or psychiatric condition that, in the judgment of the investigator, would have made the patient unsuitable for this study.
32. Treatment with an investigational drug within 3 months or 5 half-lives (whichever was longer) preceding screening.
33. Use of an active AD vaccine within 2 years before screening or monoclonal antibody for treatment of AD within 1 year before screening.
34. Participation in any other studies in which the Cogstate test battery was used, or studies involving cognitive rehabilitation either during the study or within 1 month before the screening visit.
35. Previous participation in any study of HTL0018318.

### Endpoints

The primary endpoint of this study was to evaluate the safety and tolerability of HTL0018318. Safety and tolerability were assessed via the incidence and severity of treatment-emergent adverse events (TEAEs), and via evaluation during study visits of vital signs (including blood pressure [BP] and heart rate [HR]), electrocardiogram (ECG) measurements, physical and neurological examinations, laboratory haematology, clinical chemistry and urinalysis.

Exploratory PD effects of HTL0018318 were examined via both behavioural (Cogstate neuropsychological test battery [NTB]) tests of cognitive function and electrophysiological (electroencephalogram [EEG] and evoked response potentials [ERPs]) biomarkers. Further details of each biomarker/test and outcome variables are provided below. Twelve-item neuropsychiatric inventory (NPI-12) scores were used to evaluate neuropsychiatric symptoms.

Selected plasma pharmacokinetics (PK) parameters for HTL0018318 were estimated during the period over which PD assessments were made. Details of assessment times for all endpoints are described below.

### Assessments and Analyses

Safety was the primary endpoint and was assessed at all visits (screening, baseline, Day 1, Day 6±1, Day 11±1, Day 16±1, Day 28±1, and Day 35±2 [follow-up]) based on: spontaneously reported treatment-emergent adverse events (TEAEs); scheduled physical and neurological examinations; vital sign measurements (body temperature, heart rate [HR], systolic and diastolic BP [SBP and DBP]); 12-lead ECG recordings; and clinical laboratory safety panel. The C-SSRS was administered at baseline and on Day 28±1. TEAEs were coded using the Medical Dictionary for Regulatory Activities (MedDRA) Version 20.

Exploratory pharmacodynamic endpoints (Cogstate tests and EEG/evoked response potential [ERP] biomarkers) were evaluated at screening (practice sessions) and baseline visits, and on Day 28±1. While this study was not powered to detect PD effects of small to moderate ESs, the study sample size provided approximately 80% power to detect a large ES of 0.97 for these endpoints using a significance level of 0.10. As such, results have been described as meaningful improvements if they were statistically significant (P<0.10) and had an ES>0.40.

Pharmacokinetics (PK) were evaluated via blood samples, for determination of HTL0018318 concentrations in plasma. Blood samples were collected during clinic visits before morning dose administration and 1, 2, and 4 hours after dose administration, on Day 1, Day 6, Day 11, Day 16, Day 28, and Day 35 (follow-up visit). Bioanalysis used a fully validated method and all samples were analysed within the established period of storage stability. Validated Phoenix® WinNonlin® Version 6.4 was used to derive PK parameters.

All safety analyses used the safety set (SAF; randomised dose). The SAF (randomised dose) included all randomised patients who received at least one dose of the study drug; treatment group assignment was based on the initial randomised dose. All efficacy analyses were considered exploratory and used the full analysis set (FAS). The FAS included all randomised patients with at least one post-baseline efficacy measurement; treatment group assignment was based on the initial randomised dose. All summaries and analyses of PK data used the pharmacokinetic set (PKS). The PKS included all randomised patients with valid PK assessments; treatment group assignment was based on actual dose received.

### Blood Pressure and Heart Rate Measurements

BP and HR were measured in triplicate, both supine and standing, in the same arm throughout the study. Standing BP and HR were measured after 3 minutes of standing, and supine BP and HR after 5 minutes of lying down. Measurements were repeated if clinically significant or machine/equipment errors occurred. Out-of-range BP or HR measurements were repeated at the investigator's discretion. Any confirmed clinically significant vital sign measurements were recorded as AEs. If the following measurements were obtained, instructions for additional monitoring, or stopping dosing were to be followed:
SBP>170 mmHg; DBP>110 mmHg
SBP or DBP increase >30% from Day 1 pre-dose level.

### Cogstate Neuropsychological Test Battery

A Cogstate neuropsychological test battery (NTB; http://www.Cogstate.com/) was administered as a computerised test, with the following cognitive tasks included in the battery. The total duration of the battery was approximately 30-40 minutes:
Identification (IDN)—reaction time (attention)
International shopping list immediate recall (ISL) and international shopping list-delayed recall (ISRL)—number of words recalled (episodic memory/verbal learning)
One back test (ONB)—accuracy and reaction time (working memory/executive function)
Groton maze learning (GML; forward and reverse maze learning)—accuracy (executive function)

The behavioural tests within the Cogstate NTB battery where chosen because of their ability to detect cognitive impairment in patients with AD (Lim Y Y, Harrington K, Ames D, Ellis K A, Lachovitzki R, Snyder P J, et al. J Clin Exp Neuropsychol. 2012; 34(8):853-863; Lim Y Y, Maruff P, Pietrzak R H, Ames D, Ellis K A, Harrington K, et al. Brain. 2014; 137(Pt 1):221-231) and their demonstrated sensitivities to (1) cholinergic muscarinic receptor modulation, (Nathan P J, Watson J, Lund J, Davies C H, Peters G, Dodds C M, et al. Int J Neuropsychopharmacol. 2013; 16(4):721-731; Fredrickson A, Snyder P J, Cromer J, Thomas E, Lewis M, Maruff P. The use of effect sizes to characterize the nature of cognitive change in psychopharmacological studies: an example with scopolamine. Hum Psychopharmacol. 2008; 23(5):425-436) and (2) other pro-cognitive drugs including Histamine H3 receptor antagonists in Phase 1 and 2 studies in AD (Grove R A, Harrington C M, Mahler A, Beresford I, Maruff P, Lowy M T, et al. Curr Alzheimer Res. 2014; 11(1):47-58; Nathan P J, Boardley R, Scott N, Berges A, Maruff P, Sivananthan T, et al. Curr Alzheimer Res. 2013; 10(3):240-251).

### Identification Task

The IDN is a measure of choice reaction time. In this task, an event (ie, a card turning face-up) occurs in the centre of the computer screen, and the patient must decide “YES” or “NO” as to whether or not this event meets a predefined and unchanging criterion (is the colour of the card red?). For this test, the primary dependent measure of the study was mean of the log10 transformed reaction times for correct responses.

### International Shopping List Test

The ISL and ISRL are a computer controlled verbal learning/episodic memory test. In this test, patients are read a list of 12 words. Each word is a concrete noun and describes an item of food that is found commonly in the culture/society in which testing is occurring. The examiner tells the patients “I am going to read to you a list of items I want you to get from the supermarket/store/market/shop”. After the 12 words have been read, the patient tries to recall as many of the words as they can immediately (ie, ISL immediate recall). Once they can recall no more words, the same list is read a second time with the words in the same order, after the same instruction. The process of reading the list and waiting for responses occurs 3 times. At the completion of the computerised battery (and with a minimum 20-minute delay) the patient is asked to recall as many of the items as they can from the shopping list (ISL delayed recall; ie, ISRL). For both ISL and ISRL tests, the primary dependent measure was number of words recalled.

### One Back Test

The ONB memory task is a valid measure of working memory. In this task, the patient is shown a single stimulus in the centre of the computer screen (ie, a card turns face-up). He or she must decide “YES” or “NO” as to whether or not the current card matches the card that was seen on the immediately previous trial. The software measures the speed and accuracy of each response. For this test, the primary dependent measure of the study was arcsine transformation of the proportion of correct responses (i.e. accuracy).

### Groton Maze Learning Test

The modified GML evaluates short-term memory. The test begins with a chase task to allow the patient to become familiar with the task context. The patient is shown a 10×10 grid of tiles on a computer touch screen. The patient is instructed to chase a moving tile around the grid. Once the patient understands the rules, he or she can proceed onto the timed chase test, where the patient must chase the target for 30 seconds. For the GML, the patient is shown the same 10×10 grid of tiles on a computer touch screen. A 28-step pathway is hidden among these 100 possible locations. The start is indicated by the blue tile at the top left, and the finish location is the tile with the red circles at the bottom right of the grid. The patient is instructed to find the pathway that is hidden beneath the tiles. The patient does this by selecting one tile at a time. With each choice, the computer indicates whether or not it was correct by revealing a green checkmark (ie, this is one step in the pathway). If the choice was incorrect, a red cross is shown (ie, this is not a step in the pathway). If the choice was correct, the location with the green check mark remains visible. The red cross that marks incorrect locations is hidden as soon as the next choice is made. While locating the pathway, the patient is not required to adhere to any rules; rather, the patient should just find the pathway as efficiently as he or she can. Once the patient has found all steps in the 28-step pathway, the green check marks indicating the steps in the pathway are extinguished, marking the end of the trial, and the patient is required to return to the start location and find the same pathway a second time. This process is repeated four times to yield five learning trials. Patients also have to repeat the test in the reverse direction, in which they follow the same instructions as before to find the hidden pathway but find the pathway backwards. There are 20 well-matched alternate forms for this task, and these are selected in pseudo-random order to ensure that no patient will be required to learn the same hidden path until all 20 have been completed. For this test (for both forwards and backwards parts), the primary dependent measure was number of errors made (ie, accuracy) to find the hidden pathway summed across the five trials.

### Electroencephalography and Evoked Response Potentials

Brain activity was measured at rest using EEG, and during presentation of auditory stimuli using ERPs. The following EEG and ERP parameters were examined because of their sensitivities in detecting brain activity abnormalities at rest and during cognitive processing in patients with AD (Dierks T, Ihl R, Frolich L, Maurer K. Psychiatry Res. 1993; 50(3):151-162; Hedges D, Janis R, Mickelson S, Keith C, Bennett D, Brown B L. Clin EEG Neurosci. 2016; 47(1):48-55; Pekkonen E. Audiol Neurootol. 2000; 5(3-4):216-224):
Resting state power in the delta, theta, alpha, beta and gamma frequency bands measured by EEG activity during eyes open and eyes closed conditions (3 minutes each).
ERP markers (approximately 45 minutes)
   Mismatch negativity (MMN; sensory gating/change detection)—amplitude and latency
   P300-P3a and P3b (attention and working memory updating)—amplitude and latency
   40 Hz auditory steady-state response (ASSR; gamma oscillations and synchrony)—gamma-band evoked power and phase locking

EEG and ERP markers were quantified using the following paradigms:
Resting state paradigm (6 minutes): EEGs were recorded while patients were sitting with their eyes open (3 minutes) and closed (3 minutes) for frequency band-power analyses in the delta, theta, alpha, beta, and gamma frequency bands. Primary dependent measure was mean normalised power at Cz electrode in the eyes open and closed conditions.
Passive auditory oddball paradigm (23 minutes): Patients were instructed to ignore auditory stimuli while viewing a silent movie. Patients listened to a pseudorandom sequence of 85 dB tones (5 msec rise/fall times, 500 msec stimulus onset asynchrony); 85% were standards (50 msec, 1 kHz) and 15% were deviants that differed in both duration and pitch (125 msec, 1.5 kHz). Event-related potentials were generated in response to standard and deviant waveforms. Deviant minus standard difference waves were generated. The primary dependent measures were MMN amplitude and latency at Fz and FCComp electrodes (an average of frontal electrodes).
40 Hz ASSR paradigm (8 minutes): Gamma steady-state responses were assessed in response to 1 ms 85 dB white noise clicks presented in 500 ms trains at a frequency of 40 Hz, with an inter-train interval of 1 second. A total of 500 click trains were presented. The primary dependent measure was gamma-band—evoked power and gamma phase locking at Fz and FCComp electrodes.
Active auditory oddball paradigm (11 minutes): Patients listened to a pseudorandom sequence of 85 dB tones (5 msec rise/fall times, 1500 msec stimulus onset asynchrony); 80% were standards (50 msec, 1 kHz) and 20% were targets (500 msec, 0.5 kHz). Patients were instructed to press a button when they heard target stimuli. ERPs were generated in response to target tones (P3b). The primary dependent measures were P3b amplitude and latency at CPComp electrodes (an average of central and parietal electrodes).

Patient data were collected from a 64-electrode cap onto a qualified and validated study system operated by site personnel who were specifically trained and certified for the study. The EEGs obtained at each patient visit were reviewed qualitatively by the study EEG expert (who remained blinded to dose allocation) and feedback was provided directly to the relevant site personnel on an ongoing basis and within 2 days of the assessment, to minimise issues from inter-site variability and to ensure the highest possible levels of consistency and reproducibility with respect to the EEG procedures. Once all patients had completed the EEGs and data were collected, the EEG expert and study team reviewed all data in a blinded manner using a set of predefined criteria to identify which EEG tracers were analysable and which needed to be excluded for statistical analyses.

### Statistical Analyses

Statistical evaluation was performed using Statistical analysis software (SAS®, SAS Institute, Cary, NC). Changes from baseline in vital signs were analysed using a mixed model for repeated measures with fixed effects for baseline, treatment, time-point and treatment by time-point interaction. The exploratory PD endpoints were analysed using an analysis of covariance with change from baseline as the dependent variable, and baseline and treatment as covariates. The least square means (LSM) for each dose (and difference from placebo) are presented with the 90% CI, P-value, and ES. ES was calculated as the LSM difference from placebo divided by the pooled standard deviation. PD data were considered clinically meaningful when P-values were <0.10 and ESs were >0.4 (ie, moderate to large ES).

It was estimated that a sample size of 60 patients (15 per treatment group, with a is continuation rate of up to 10%) could provide ≥80% power, assuming a true standardised ES of at least 0.97 in each active group versus placebo in mean change from baseline in EEG-ERP to an auditory stimulus. Testing was assumed with a two-sided significance level of 0.10 and without adjustment for multiple comparisons.

### Results

### Patients

Eighty-seven patients were screened, and 60 were randomised to receive at least one dose of the study drug (placebo, n=15; HTL0018318 5 mg, n=15; 15 mg, n=14; 25 mg, n=16; safety set [SAF, randomised dose], PK set [PKS]; FIG. 4). The full analysis set (FAS) comprised 59 patients, with one patient excluded from the 5 mg group. All patients receiving placebo and 93.5% of patients (43/45) receiving HTL0018318 completed the study. Median treatment compliance was 100% in all arms. Baseline demographics and clinical characteristics were generally comparable across treatment groups (Table 9).

### Safety and Tolerability

TEAEs reported in patients receiving placebo or HTL0018318 are shown in Table 10; headache was the most commonly reported TEAE across all treatment groups. The majority of TEAEs were mild in severity and occurred during the titration phase (TEAE incidence was ˜30% lower during the dose maintenance phase). There were no serious TEAEs or deaths. Two patients discontinued treatment due to TEAEs (increased BP in one patient randomised to 5 mg, on Day 1 of dosing; nausea in one patient randomised to 15 mg, on Day 24 of dosing). TEAE incidence (but not treatment-related TEAE incidence) increased with increasing dose of HTL0018318. Cholinergic TEAEs included abdominal pain, diarrhoea, fatigue, and nausea, with incidences of 0-13% at the two highest HTL0018318 doses (15 and 25 mg).

Post-dose increases in systolic BP (SBP) and diastolic BP (DBP) were observed in patients receiving HTL0018318 (all doses, without a dose-relationship), generally returning to pre-dose levels after a few hours (FIGS. 12-15); pre-dose BP did not increase progressively with continued dosing. Post-dose decreases in SBP and DBP were observed in patients receiving placebo. Mixed model statistical analysis showed that the maximum mean elevations in SBP and DBP in patients receiving HTL0018318 were 5-10 mmHg above mean values in patients receiving placebo and occurred at time of maximum concentration (Tₘₐₓ; 1-2 hours post-dose; FIGS. 6-9). The drug effect appeared similar across all HTL0018318 doses, and was only observed on Days 1, 6, and 11, with no differences between patients receiving placebo and HTL0018318 on Days 16 and 28. There were no significant changes in orthostatic BP or HR with HTL0018318 (across doses) or placebo (FIGS. 6-9, 12-15).

There were no significant or consistent patterns in changes in ECG profiles or other vital signs across treatment groups or study days. There were no clinically significant physical or neurological examination findings, or laboratory abnormalities, including liver function and haematology, and no increased risk of suicide.

### Exploratory Pharmacodynamic Endpoints: Cogstate Neuropsychological Test Battery

Significant improvements in attention performance (reaction time; IDN) were observed at the HTL0018318 15 mg dose (LSM difference 0.11; 90% CI: 0.02, 0.21; P=0.0455; ES 0.62) (FIG. 10). Non-significant, improvements were observed at the 5 and 25 mg doses.

Clinically important improvements in learning and memory were observed with HTL0018318 25 mg (ISL-immediate recall: LSM difference 1.11 words; 90% CI: -1.04, 3.25; P=0.39; ES 0.34; ISL-delayed recall: LSM difference 0.65; 90% CI: −0.25, 1.56; P=0.2330; ES 0.49; composite [ISL-immediate and -delayed recall]: LSM difference 0.21, 90% CI: −0.09, 0.51; P=0.2421; ES 0.48) (FIG. 10). However, the magnitude of these benefits was too small to yield statistical significance.

There were no statistically significant differences in ONB reaction time or accuracy, or GML task (forward or reverse) performance, at any HTL0018318 dose (FIG. 10).

### Exploratory Pharmacodynamic Endpoints: EEG and ERP

### Resting State Paradigm

A significant increase in delta power at the central (Cz) electrode in the eyes open condition was observed with the 5 and 15 mg doses of HTL0018318, with a small effect in the same direction observed with the 25 mg dose; the largest ES was observed with the 15 mg dose (LSM difference 3.8; 90% CI: 0.6, 7.0; P=0.053; ES 0.75). There were no other significant effects on EEG power across the different frequency bands in the eyes open or closed conditions at any dose.

### Passive Auditory Oddball Paradigm

A significant improvement in MMN amplitude (ie, more negative) at the frontal (Fz) electrode was observed with the 5 and 15 mg doses of HTL0018318, with a small effect in the same direction observed with 25 mg (FIG. 4A). The ES was large with the 5 mg dose (LSM difference −0.82 uV; 90% CI: −1.43, −0.20); P=0.030; ES −0.84), and moderate with the 15 mg dose (LSM difference −0.63uV; P=0.105; ES −0.64). Improvements in MMN amplitude and peak MMN amplitude using the frontal central composite (FCComp) electrode were consistent with those observed at the Fz electrode. No statistically significant improvements were observed in peak MMN latency.

No consistent statistically significant effects were observed on P3a amplitude or latency at the Fz electrode or FCComp across the three HTL0018318 doses. However, a significant decrease in P3a amplitude at the Fz electrode was observed with the 15 mg dose (LSM difference −0.84 uV; 90% CI: −1.59, −0.09; P=0.065; ES −0.73); the effects with 5 mg and 25 mg were not consistent with this.

### Active Auditory Oddball Paradigm

There were no statistically significant differences in P3b mean amplitude at the central parietal composite (CPComp) electrodes with any HTL0018318 dose. However, consistent increases in P3b mean amplitude were observed across all 3 doses (ESs: 0.22-0.44; FIG. 4B).

There were statistically significant decreases in peak P3b latency of moderate to large magnitude at the CPComp electrodes with the 5 and 25 mg doses of HTL0018318 (FIG. 4B). The largest effect was with 5 mg (LSM difference −38 ms; 90% CI: −70, −6.1; P=0.052; ES −0.81). There was no significant decrease with the 15 mg dose.

### Hz Auditory Steady-State Response Paradigm

There were no significant effects with any HTL0018318 dose in amplitude of gamma-band evoked power or phase locking at the Fz electrode across all time bands/window (ie, 1-500 ms).

### Exploratory Pharmacodynamic Endpoints: Neuropsychiatric Inventory

There was a statistically significant reduction in NPI-12 symptoms (total score) at the 5 mg HTL0018318 dose (LSM difference −1.79; 90% CI: −3.31, −0.26; P=0.055; ES -0.73) compared with placebo, with consistent trends observed at the 15 and 25 mg doses (ES −0.54 and −0.53, respectively). Overall, the incidence of symptoms was low across patients (mean scores 2-2.5 [range 0-26]).

### Exploratory Endpoints: Pharmacokinetics

Plasma PK of HTL0018318 was used to establish the dose-exposure relationship as patients progressed through the up-titration scheme (Table 11). Once established on any given dose level, PK did not change on subsequent PK sampling days, showing that steady-state was achieved with 5 days of daily dosing. Pre-dose concentrations, reflecting trough concentrations at steady-state, showed the mean minimum concentration above which HTL0018318 was sustained throughout the dosing interval. Dose-exposure proportionality was confirmed on Day 28. On Day 28, HTL0018318 mean maximum concentration (C max) was 51.8 ng/mL in patients receiving 5 mg, 89.0 ng/mL for 10 mg, 123 ng/mL for 15 mg, and 224 ng/mL for 25 mg. Likewise, on Day 28, mean area under the curve from time 0 to 4 hours post-dose (AUC₀₋₄ h) was 144 h·ng/mL with 5 mg, 222 h·ng/mL with 10 mg, 368 h·ng/mL with 15 mg and 668 h·ng/mL with 25 mg. Median T max ranged from 1 to 2 hours for all doses.

### Discussion

This Phase 1b/2a study is the first investigation of the safety, tolerability, PK, and exploratory PD effects (cognition and neuropsychiatric symptoms) of the selective muscarinic M₁ receptor orthosteric agonist HTL0018318, in patients with mild-to-moderate AD.

HTL0018318 was up-titrated over a 2-week period adjunctive with a stable dose of donepezil during a 4-week study.

HTL0018318 exhibited reproducible PK, consistent with previous studies in healthy young and elderly participants (Bakker C, Tasker T, Liptrot J, Hart E, Klaassen E, Doll R J, Brown G A, Brown A J H, Congreve M, Weir M, Marshall F H, Cross D M, Groeneveld G, Nathan P J. Alzheimers Res Ther. 2020; (submitted); Bakker C, Tasker T, Liptrot J, Hart E P, Klaassen E S, Prins S, et al. Br J Clin Pharmacol. 2020). Systemic exposure to HTL0018318 (as indexed by C max and AUC₀₋₄ h) increased proportionally across 5-25 mg doses and did not change on repeat dosing. HTL0018318 was generally well tolerated, with only two patients discontinuing treatment and the majority of TEAEs being mild and infrequent; the safety profile was generally consistent with that reported in previous studies (Bakker C, Tasker T, Liptrot J, Hart E, Klaassen E, Doll R J, Brown G A, Brown A J H, Congreve M, Weir M, Marshall F H, Cross D M, Groeneveld G, Nathan P J. Alzheimers Res Ther. 2020; (submitted); Bakker C, Tasker T, Liptrot J, Hart E P, Klaassen E S, Prins S, et al. Br J Clin Pharmacol. 2020). The most common TEAEs were dose related and included abdominal pain, diarrhoea, fatigue, headache, hyperhidrosis and nausea; only headache occurred in >2 patients taking HTL0018318. Of note, incidences of cholinergic TEAEs were a maximum of 7 or 13% at the two highest doses (15 and 25 mg). It has previously been suggested that M₁ PAMs may have a better cholinergic TEAE profile than M₁ receptor agonists (Bradley S J, Molloy C, Bundgaard C, Mogg A J, Thompson K J, Dwomoh L, et al. Mol Pharmacol. 2018; 93:645-56). However, the incidence of cholinergic adverse events was 21% in a recent clinical study of the M₁ PAM MK-7622 in patients with mild-to-moderate AD, (Voss T, Li J, Cummings J, Farlow M, Assaid C, Froman S, et al. Alzheimer's & dementia (New York, N Y). 2018; 4:173-81) higher than the 7-13% incidences reported with the two highest doses of HTL0018318 in the current study. Interestingly, the incidence of TEAEs appeared to be approximately 30% less during the dose maintenance (Days 16-28) versus up-titration (Days 1-15) period. While previous clinical experience did not indicate that an up-titration regimen was necessary to manage TEAEs, this approach may have contributed to the low TEAE incidence and mild TEAE profile (overall and cholinergic TEAEs specifically), tolerating out in the current study.

HTL0018318 was associated with transient increases in BP, with the maximum mean increase of 5-10 mmHg in SBP and DBP observed around the estimated time of highest systemic drug exposure, without a clear dose-response relationship and with some evidence for tolerance with continued dosing. While the exact mechanism associated with the transient increase in BP is not known, it is likely that it is mediated through central activation of M₁ receptors (Brezenoff H E, Giuliano R. Annu Rev Pharmacol Toxicol. 1982; 22:341-81; Brezenoff H E, Xiao Y F. Life Sci. 1989; 45:1163-70; Scheucher A, Pirola C J, Balda M S, Dabsys S M, Alvarez A L, Finkielman S, et al. Neuropharmacol. 1987; 26:181-5; Medina A, Bodick N, Goldberger A L, Mac Mahon M, Lipsitz L A. 1997; 29:828-34). No significant treatment effects were observed on other cardiovascular endpoints (including orthostatic BP, HR and ECG) or clinical laboratory safety parameters. The increase in BP with no significant effects on orthostatic BP or HR contrasts with other muscarinic agonists, including the M₁/M₄ agonist xanomeline, which caused significant increases in BP and HR, as well as a decrease in orthostatic BP (Scheucher A, Pirola C J, Balda M S, Dabsys S M, Alvarez A L, Finkielman S, et al. Neuropharmacol. 1987; 26:181-5). Our data suggest that the cardiovascular effects of muscarinic agonists in AD may be less pronounced with a partial agonist and when dosed using a titration regimen within the dose range tested here.

Exploratory PD effects were measured using both behavioural and electrophysiological biomarkers of cognitive function. The behavioural tests within the Cogstate NTB are sensitive to cholinergic modulation (Fredrickson A, Snyder P J, Cromer J, Thomas E, Lewis M, Maruff P. Human psychopharmacology. 2008; 23:425-36) and are able to detect cognitive impairment in patients with AD (Lim Y Y, Harrington K, Ames D, Ellis KA, Lachovitzki R, Snyder P J, et al. J Clin Exp Neuropsychol. 2012; 34:853-63; Lim Y Y, Maruff P, Pietrzak R H, Ames D, Ellis K A, Harrington K, et al. Brain. 2014; 137:221-31). In addition, the Cogstate NTB has been shown to be more sensitive in detecting pro-cognitive signals after short durations of treatment than other measures, such as the Alzheimer's Disease Assessment Scale—Cognition (ADAS-Cog) (Grove R A, Harrington C M, Mahler A, Beresford I, Maruff P, Lowy M T, et al. Current Alzheimer research. 2014; 11:47-58). The EEG and ERP tasks evaluated very early sensory processing related to resting state brain activity (EEG power in various frequency bands), attention and memory (MMN), network activity and synchrony in the gamma frequency range (40 Hz ASSR), attention (P3a), and attention/working memory (P3b). These biomarkers have been shown to detect cognitive deficits in patients with AD (Dierks T, Ihl R, Frolich L, Maurer K. Psychiatry Res. 1993; 50:151-62; Hedges D, Janis R, Mickelson S, Keith C, Bennett D, Brown B L. Clin EEG Neurosci. 2016; 47:48-55; Audiol Neurootol. 2000; 5:216-24).

HTL0018318 showed positive PD effects on a number of cognitive biomarkers, providing evidence of central target engagement and clinically relevant effects on cognitive function. On the Cogstate NTB, a meaningful improvement in attention was observed, along with encouraging data for episodic memory (HTL0018318 25 mg: ISL-delayed recall, ES 0.49; ISL composite, ES 0.48). The improvement in episodic memory is consistent with pre-clinical evidence across multiple muscarinic M₁ receptor agonists, as well as previous findings in humans using the same test with the M₁ agonist GSK1034702 (Nathan P J, Watson J, Lund J, Davies C H, Peters G, Dodds C M, et al. The international journal of neuropsychopharmacology. 2013; 16:721-31). The cognitive improvements with HTL0018318 are clinically relevant in the context of the impairments in attention and episodic memory observed in mild cognitive impairment (MCI) and AD (ES 0.5-2.5) (Lim Y Y, Harrington K, Ames D, Ellis K A, Lachovitzki R, Snyder P J, et al. J Clin Exp Neuropsychol. 2012; 34:853-63; Lim Y Y, Maruff P, Pietrzak R H, Ames D, Ellis K A, Harrington K, et al. Brain. 2014; 137:221-31). In addition, cognitive interventions (ie, cognitive remediation) with an ES of 0.4 have shown clinical benefit (McGurk S R, Twamley E W, Sitzer D I, McHugo G J, Mueser K T. The American journal of psychiatry. 2007; 164:1791-802).

Consistent with the effects of HTL0018318 on cognitive performance, meaningful and clinically relevant effects were also observed in electrophysiological biomarkers. Effects were observed for MMN and eyes open delta power; mean ESs (all HTL0018318 doses) were moderate (0.6 and 0.52, respectively). MMN is a biomarker of early sensory attention and memory and change detection. Impairments in MMN (both amplitude and latency) have been reported in MCI and AD (Pekkonen E, Jousmaki V, Kononen M, Reinikainen K, Partanen J. Neuroreport. 1994; 5:2537-40; Lindin M, Correa K, Zurron M, Diaz F. Frontiers in aging neuroscience. 2013; 5:79), with impairments correlated with episodic memory (Laptinskaya D, Thurm F, Kuster O C, Fissler P, Schlee W, Kolassa S, et al. Frontiers in aging neuroscience. 2018; 10:5). While the effects of cholinesterase inhibitors (such as donepezil) on MMN are not known, the magnitude of effects observed in this study in AD are similar or larger than the magnitude of effects reported with the cognitive enhancer memantine in various clinical populations (Korostenskaja M, Nikulin V V, Kicic D, Nikulina A V, Kahkonen S. Brain Res Bull. 2007; 72:275-83; Swerdlow N R, Bhakta S, Chou H H, Talledo J A, Balvaneda B, Light G A. Neuropsychopharmacol. 2016; 41:419-30). The P3b is a marker of attention and memory, and changes in P3b reflect the amount (and speed) of attentional resources allocated when working memory is updated (Polich J, Criado J R. International journal of psychophysiology: official journal of the International Organization of Psychophysiology. 2006; 60:172-85). Consistent impairments in P3b of moderate to large magnitudes have been reported in MCI and AD for both amplitude and latency, with positive associations found between P3b latency changes and various cognitive processes (Howe A S, Bani-Fatemi A, De Luca V. Brain Cogn. 2014; 86:64-74; Thomas A, Iacono D, Bonanni L, D'Andreamatteo G, Onofrj M. Clinical neuropharmacology. 2001; 24:31-42). The moderate improvements in P3b amplitude and latency observed in this study (ESs 0.33 and 0.42) are larger than those reported for cholinesterase inhibitors such as donepezil and rivastigmine (ES of 0.08-0.21) (Thomas A, Iacono D, Bonanni L, D'Andreamatteo G, Onofrj M. Clinical neuropharmacology. 2001; 24:31-42). The significance of delta power changes is unknown, although slow oscillations in the delta and theta band are thought to be effective in activating long-range network states associated with cognitive function, including memory (Rac-Lubashevsky R, Kessler Y. Journal of cognitive neuroscience. 2018; 30:1870-82). In this context, the observed increases in delta power are consistent with the effects observed on MMN and P3b, as well as the behavioural tasks that measured attention and episodic memory.

The PD effects of HTL0018318 on biomarkers of cognition provide compelling evidence of preliminary support for central target engagement in patients with AD maintained on therapeutic doses of donepezil. Clinically meaningful procognitive effects of moderate to large magnitude were observed in early sensory attentional and memory processing (MMN), attention (P3b and IDN) and episodic memory (ISL), but not in executive function (ONB and GML). It is possible that higher doses may be required to improve executive function, which requires more complex and effortful cognitive resources. Overall, these findings are encouraging for a number of reasons. First, as word recognition/recall constitutes almost 45% of the ADAS-Cog total score, the improvements observed in ISL performance may translate into improvements in ADAS-Cog performance in future Phase 2/3 studies. Secondly, the duration of treatment was only 4 weeks; larger effects may be observed over a longer treatment duration, when symptomatic effects may be more apparent against normal cognitive decline. Finally, the moderate to large ESs observed across cognitive biomarkers reflect benefits that occurred on top of therapeutic doses of donepezil, which adds further significance to the findings. Interestingly, HTL0018318 also showed some positive effects of moderate magnitude on neuropsychiatric symptoms as measured by the NPI-12. These data are encouraging and support findings previously reported with the M₁/M₄ receptor agonist xanomeline in AD (Bodick NC, Offen WW, Shannon HE, Satterwhite J, Lucas R, van Lier R, et al. Alzheimer disease and associated disorders. 1997;11(suppl. 4):516-22).

Similar increases in blood pressure were observed across all HTL0018318 doses, and there was no clear differentiation between doses in PD effects. Evidence of effects on MMN, P3b and Cogstate Identification was found across all HTL0018318 doses, although dose-response relationships differed between endpoints; Type I and Type II errors in pairwise dose comparisons with placebo may explain some of these differences. The optimal dose for AD and other indications will be explored in future studies; considering that HTL0018318 was well tolerated at the top dose, it would be feasible and informative to assess safety and PD effects using a wider dose range than 5-25 mg. A limitation of this study was that due to the small number of patients receiving concomitant memantine (n=4), we could not draw conclusions on the tolerability or PD effects of the combination of HTL0018318 with memantine.

In summary, this 4-week, Phase 1b/2a study demonstrated that the M₁ receptor orthosteric agonist HTL0018318 was well tolerated in patients with mild-to-moderate AD when administered as an adjunctive treatment to stable doses of donepezil using a 2-week titration regimen. HTL0018318 also showed positive and clinically meaningful effects on biomarkers of cognitive function. These findings provide support for further development of HTL0018318 as a symptomatic treatment of dementias including AD.

TABLE 9
Baseline demographics and clinical characteristics (SAF, randomised)

| | Placebo | | HTL0018318 | | HTL0018318 | | HTL0018318 | |
|---|---|---|---|---|---|---|---|---|
| | (n = 15) | | 5 mg (n = 15) | | 15 mg (n = 14) | | 25 mg (n = 16) | |
| Age, mean (SD) | 72.1 | (8.5) | 72.2 | (6.4) | 71.1 | (4.3) | 73.5 | (8.9) |
| Female, n (%) | 10 | (66.7) | 9 | (60.0) | 11 | (78.6) | 11 | (68.8) |
| White race, n (%) | 15 | (100) | 15 | (100) | 14 | (100) | 16 | (100) |
| BMI (kg/m²), mean (SD) | 27.0 | (3.3) | 27.1 | (3.8) | 26.6 | (3.4) | 25.9 | (4.6) |
| Years since AD | 2.9 | (2.5) | 3.6 | (2.4) | 2.4 | (2.1) | 2.9 | (1.5) |
| diagnosis, mean (SD) | | | | | | | | |
| MMSE, mean (SD) | 20.5 | (3.5) | 21.5 | (1.8) | 20.6 | (3.1) | 20.8 | (2.5) |
| Donepezil use, n (%) | 15 | (100) | 14 | (100) | 14 | (100) | 16 | (100) |
| Memantine use^{a}, n (%) | 1 | (6.7) | 1 | (6.7) | 1 | (7.1) | 1 | (6.3) |
| Country, n (%) | | | | | | | | |
| Czech Republic | 3 | (20.0) | 3 | (20.0) | 2 | (14.3) | 4 | (25.0) |
| Poland | 1 | (6.7) | 2 | (13.3) | 3 | (21.4) | 4 | (25.0) |
| Slovakia | 6 | (40.0) | 7 | (46.7) | 6 | (42.9) | 6 | (37.5) |
| Spain | 5 | (33.3) | 3 | (20.0) | 3 | (21.4) | 2 | (12.5) |

^{a}In Poland, use of memantine was exclusionary.
AD, Alzheimer's disease; BMI, body mass index; MMSE, mini-mental state examination; SAF, safety set; SD, standard deviation.

TABLE 10
Incidences of TEAEs, SAEs, and TEAEs leading to discontinuation
(SAF, randomised); B Timing of TEAEs (SAF, Day 16 dose received)^{a}
A

| | Placebo | HTL0018318 | HTL0018318 | HTL0018318 |
|---|---|---|---|---|
| | (n = 15) | 5 mg (n = 15) | 15 mg (n = 14) | 25 mg (n = 16) |
| Number of TEAEs, n | 12 | 18 | 16 | 26 |
| TEAE incidence, n (%) | 6 (40.0) | 8 (53.3) | 8 (57.1) | 11 (68.8) |
| Severity: Mild | 6 (40.0) | 7 (46.7) | 8 (57.1) | 9 (56.3) |
| Severity: Moderate | 0 | 1 (6.7) | 1 (7.1) | 4 (25.0) |
| Severity: Severe | 0 | 1 (6.7) | 0 | 0 |
| Individual TEAE incidences,^{b} n (%) | | | | |
| Abdominal pain | 0 | 1 (6.7) | 0 | 0 |
| Accidental overdose | 0 | 1 (6.7) | 1 (7.1) | 0 |
| Decreased lymphocyte count | 0 | 0 | 1 (7.1) | 1 (6.3) |
| Decreased white blood cell count | 0 | 0 | 1 (7.1) | 1 (6.3) |
| Diarrhoea | 1 (6.7) | 1 (6.7) | 0 | 1 (6.3) |
| Fatigue | 0 | 0 | 0 | 2 (12.5) |
| Headache | 3 (20.0) | 1 (6.7) | 3 (21.4) | 3 (18.8) |
| Hyperhidrosis | 0 | 0 | 2 (14.3) | 0 |
| Lymphopenia | 0 | 0 | 1 (7.1) | 1 (6.3) |
| Nausea | 0 | 1 (6.7) | 1 (7.1) | 0 |
| Urinary tract infection | 1 (6.7) | 1 (6.7) | 0 | 1 (6.3) |
| Viral upper respiratory tract | 0 | 1 (6.7) | 0 | 1 (6.3) |
| infection | | | | |
| Treatment-related^{c} TEAE | 2 (13.3) | 4 (26.7) | 7 (50.0) | 5 (31.3) |
| incidence, n (%) | | | | |
| TEAE leading to discontinuation | 0 | 1 (6.7)^{d} | 1 (7.1)^{e} | 0 |
| incidence, n (%) | | | | |
| SAE incidence, n (%) | 0 | 0 | 0 | 0 |

B

| | Placebo | HTL0018318 | HTL0018318 | HTL0018318 | HTL0018318 |
|---|---|---|---|---|---|
| | (n = 15) | 5 mg (n = 17) | 10 mg (n = 1) | 15 mg (n = 16) | 25 mg (n = 11) |
| TEAE incidence | 6 (40.0) | 8 (47.1) | 1 (100) | 8 (50.0) | 6 (54.5) |
| during titration | | | | | |
| (Days 1-15), n (%) | | | | | |
| TEAE incidence | 1 (6.7) | 4 (23.5) | 0 | 2 (12.5) | 2 (18.2) |
| during maintenance | | | | | |
| (Days 16-28), n (%) | | | | | |

Bold text highlights TEAEs potentially related to cholinergic stimulation.
^{a}Patients grouped according to the dose that they were taking at the Week 16 visit.
^{b}TEAEs potentially related to cholinergic stimulation or occurring in ≥2 patients receiving HTL0018318 and/or in ≥2 patients in any HTL0018318 dose group.
^{c}TEAEs considered by the investigator to be definitely or possibly related to the study drug at the time of the event.
^{d}Patient experienced a severe drug-related increase in BP and met stopping criteria on Day 1.
^{e}Patient experienced mild drug-related nausea leading to study drug discontinuation on Day 24.
SAE: serious adverse event; SAF, safety set; TEAE: treatment-emergent adverse event.

TABLE 11
HTL0018318 plasma PK parameters (PKS)

| PK | Visit | HTL0018318 | HTL0018318 | HTL0018318 | HTL0018318 |
|---|---|---|---|---|---|
| parameter | day | 5 mg | 10 mg | 15 mg | 25 mg |
| Cₘₐₓ (ng/mL), | 1 | n = 44 | NA | NA | NA |
| arithmetic | | 40.3 ± 24.4 | | | |
| mean ± SD | 6 | n = 17 | n = 12 | n = 14 | NA |
| | | 50.5 ± 15.6 | 82.4 ± 31.6 | 106 ± 54.0 | |
| | 11 | n = 14 | n = 2 | n = 16 | n = 11 |
| | | 48.3 ± 19.6 | 69.7 ± 33.6 | 126 ± 29.3 | 222 ± 102 |
| | 16 | n = 15 | n = 1 | n = 16 | n = 11 |
| | | 46.4 ± 18.9 | 87.5 | 136 ± 31.4 | 243 ± 108 |
| | 28 | n = 15 | n = 1 | n = 16 | n = 11 |
| | | 51.8 ± 30.7 | 89.0 | 123 ± 29.3 | 223 ± 134 |
| C_{trough} (ng/mL), | 6 | n = 17 | n = 13 | n = 14 | NA |
| arithmetic | | 14.2 ± 12.1 | 7.55 ± 4.72 | 19.0 ± 25.7 | |
| mean ± SD | 11 | n = 14 | n = 2 | n = 16 | n = 11 |
| | | 13.7 ± 10.3 | 5.47 | 27.9 ± 40.7 | 22.2 ± 21.8 |
| | | | [2.50-8.40]^{a} | | |
| | 16 | n = 16 | n = 1 | n = 16 | n = 11 |
| | | 14.1 ± 10.8 | 8.25 | 29.4 ± 38.5 | 66.7 ± 80.4 |
| | 28 | n = 16 | n = 1 | n = 16 | n = 11 |
| | | 14.2 ± 12.3 | 6.41 | 23.2 ± 16.0 | 40.3 ± 30.6 |
| AUC_{0-4 h} | 1 | n = 44 | NA | NA | NA |
| (h · ng/mL), | | 113 ± 57.6 | | | |
| arithmetic | 6 | n = 17 | n = 12 | n = 14 | NA |
| mean ± SD | | 146 ± 53.1 | 244 ± 88.0 | 315 ± 163 | |
| | 11 | n = 14 | n = 2 | n = 16 | n = 11 |
| | | 149 ± 70.6 | 231 [137-326]^{a} | 379 ± 77.5 | 650 ± 302 |
| | 16 | n = 15 | n = 1 | n = 16 | n = 11 |
| | | 132 ± 56.2 | 302 | 422 ± 94.5 | 728 ± 359 |
| | 28 | n = 15 | n = 1 | n = 16 | n = 11 |
| | | 144 ± 52.7 | 222 | 368 ± 106 | 668 ± 406 |

Data were presented to three significant figures, as arithmetic mean ± SD except ^{a}as mean [minimum, maximum] for n = 2.
Abbreviations: AUC_{0-4 h}, area under the curve from time 0 until 4 hours post-dose; Cₘₐₓ, maximum concentration; C_{trough}, trough concentration (taken pre-dose at steady-state); n, number of patients with available data; NA, not applicable; PK, pharmacokinetic; SD, standard deviation.

### BRIEF DESCRIPTION OF THE FIGURES

FIG. 1: Passive avoidance test of Example B.

FIG. 2: Study design of Example C (CRU=clinical research unit; Dpz=donepezil; ss=steady state).

FIG. 3: Timing of donepezil PK samples in Example C.

FIG. 4: Example D patient disposition flow diagram (AE=adverse event).

FIG. 5: Example D—A. Study design. B. Titration schedule (*down=5 mg dose reduction).

FIG. 6: Example D—Difference in vital signs between patients randomised to HTL0018318 versus placebo—A. Supine systolic blood pressure.

FIG. 7: Example D—Difference in vital signs between patients randomised to HTL0018318 versus placebo—B. Supine diastolic blood pressure.

FIG. 8: Example D—Difference in vital signs between patients randomised to HTL0018318 versus placebo—C. Orthostatic difference in systolic blood pressure.

FIG. 9: Example D—Difference in vital signs between patients randomised to HTL0018318 versus placebo—D. Supine heart rate (SAF, randomised).

FIG. 10: Example D—Cogstate NTB ES in patients receiving HTL0018318 compared with placebo (FAS). (*The effect in the 5 mg HTL0018318 group was driven by a single patient, who made a very large number of errors during the baseline assessment, and substantially fewer at Day 28. ES: effect size; NTB: neuropsychological test battery).

FIG. 11: Example D—EEG and ERP differences between patients receiving HTL0018318 and placebo during a passive and active auditory oddball paradigm. A. Passive auditory oddball paradigm: Head maps show drug-placebo differences in MMN amplitude. Cooler colours show greater drug related increases in MMN amplitude. Chart shows effect size of change in MMN amplitude in patients receiving HTL0018318 compared with placebo at the Fz electrode. B. Active auditory oddball paradigm: Charts show effect sizes on mean P3b amplitude and peak latency changes* in patients receiving HTL0018318 compared with placebo at the CPComp electrode (*Shortening of peak latency represents a positive treatment effect and is thus shown as a positive effect size in the chart. CPComp: central parietal composite electrode; EEG: electroencephalogram; ERP: evoked response potential; Fz: frontal (electrode); MMN: mismatch negativity).

FIG. 12: Example D—Supine systolic blood pressure (SE, standard error).

FIG. 13: Example D—Supine diastolic blood pressure (SE, standard error).

FIG. 14: Example D—orthostatic difference in systolic blood pressure (SE, standard error).

FIG. 15: Example D—Supine heart rate (SAF, randomised) (SE, standard error; bpm, beats per minute).

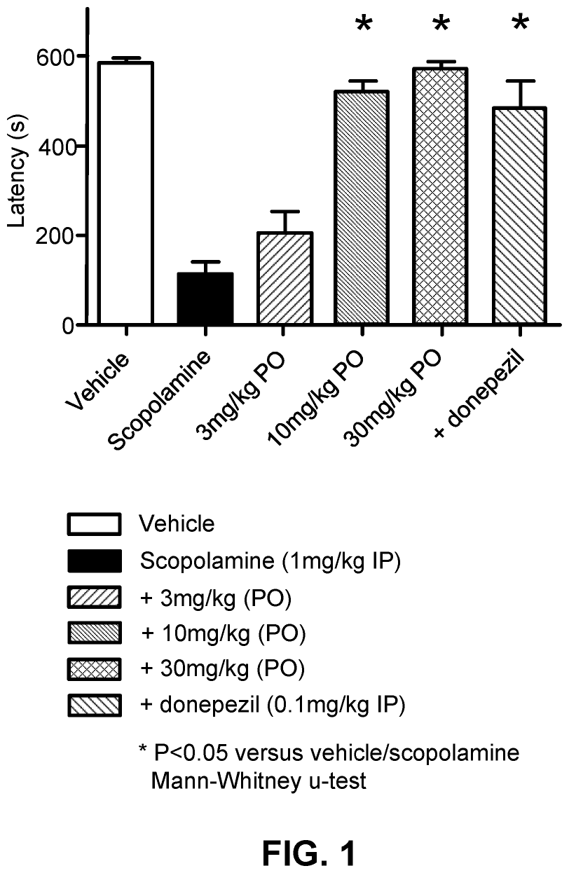

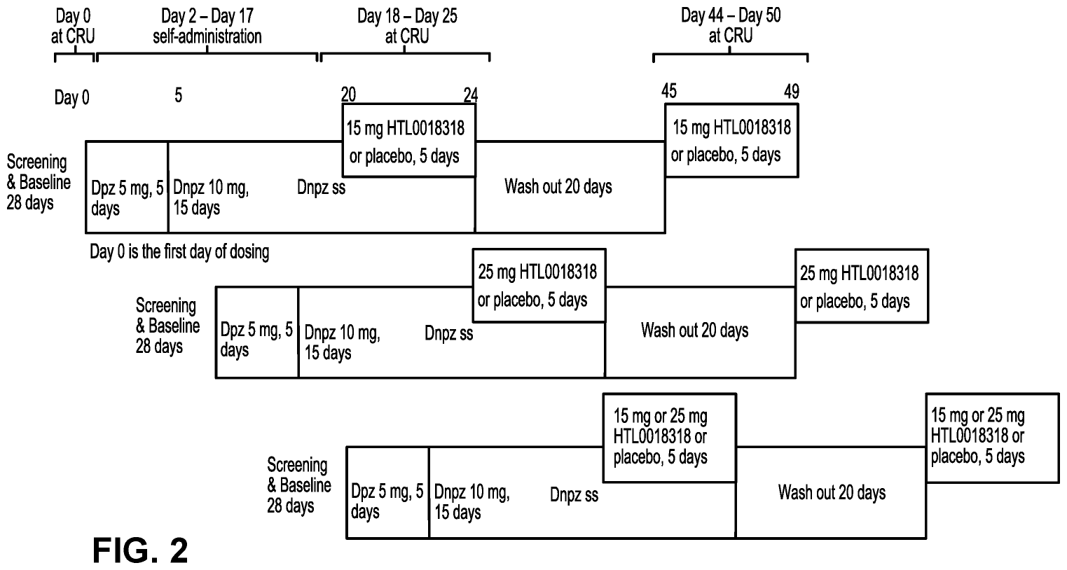

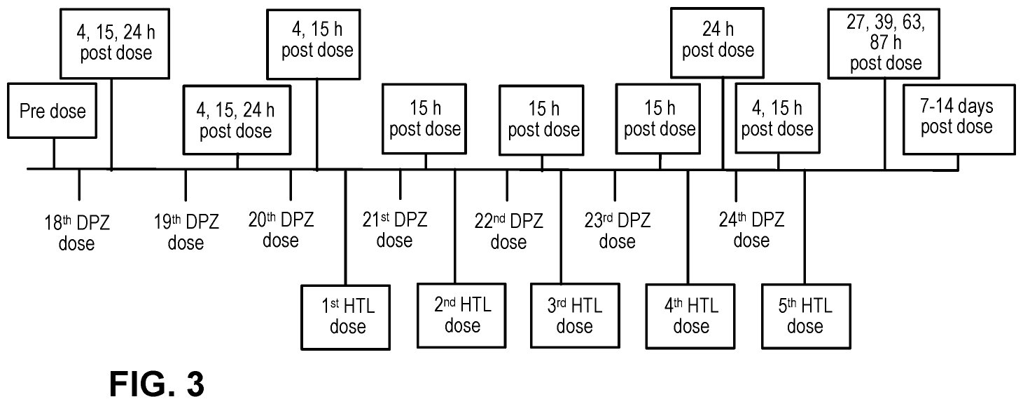

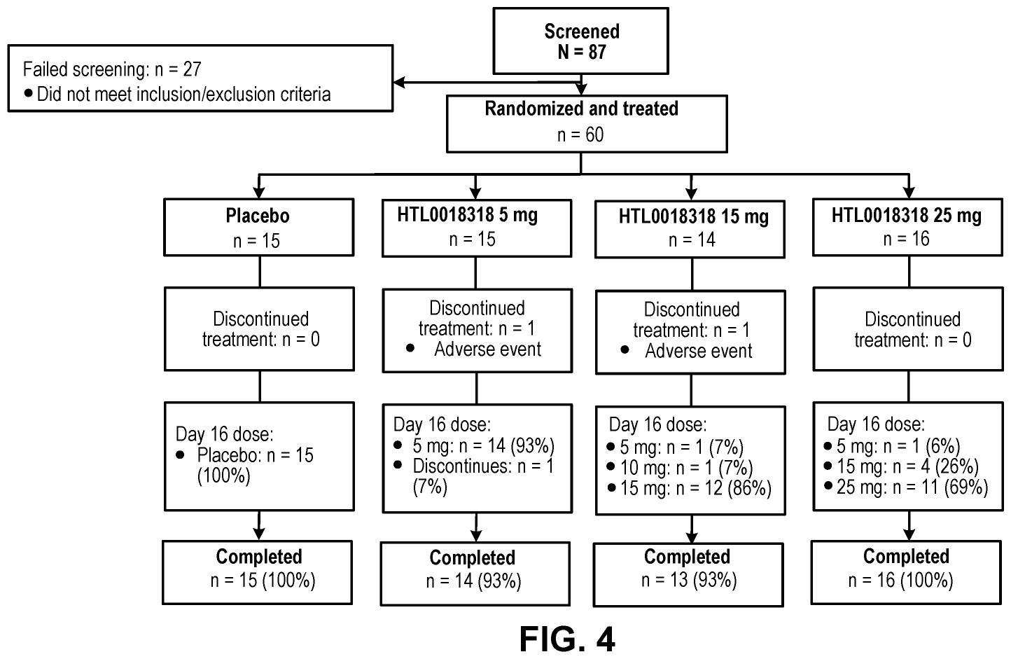

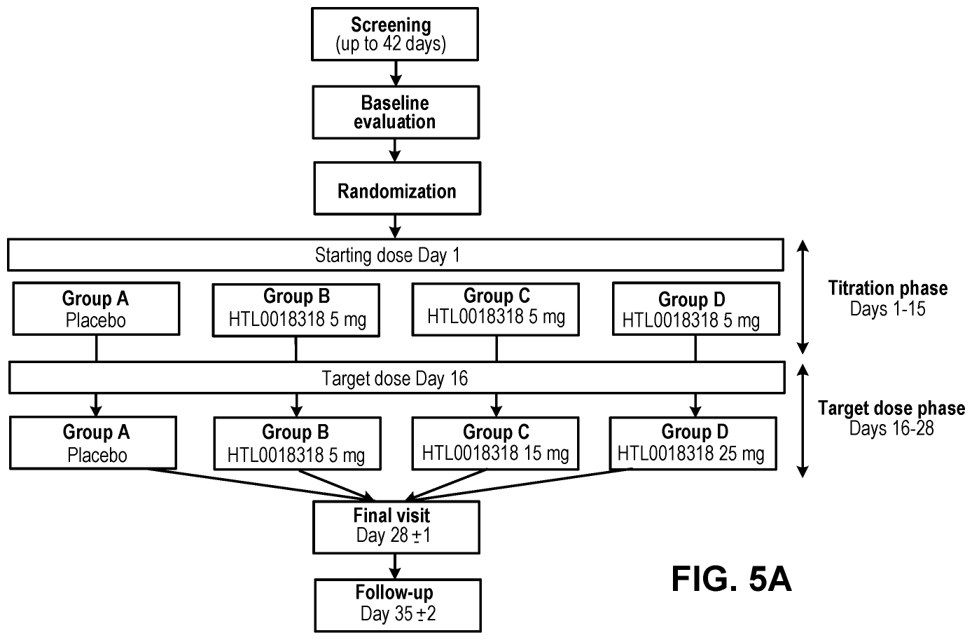

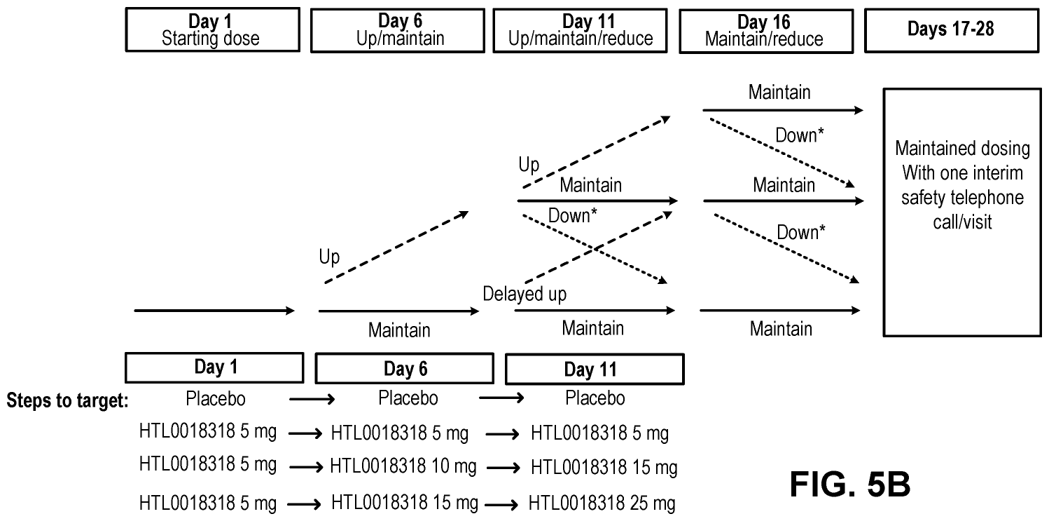

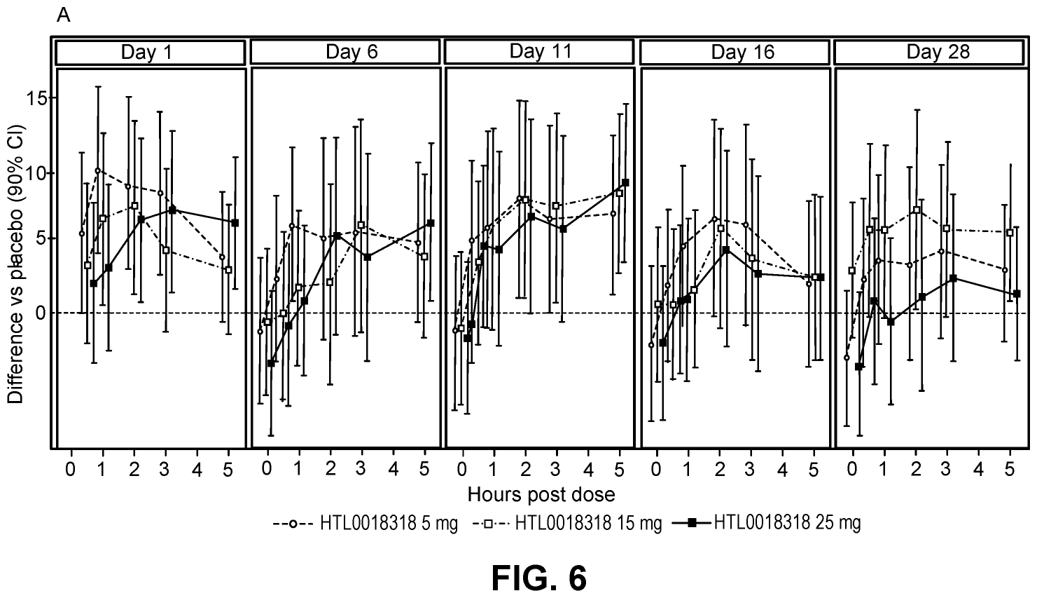

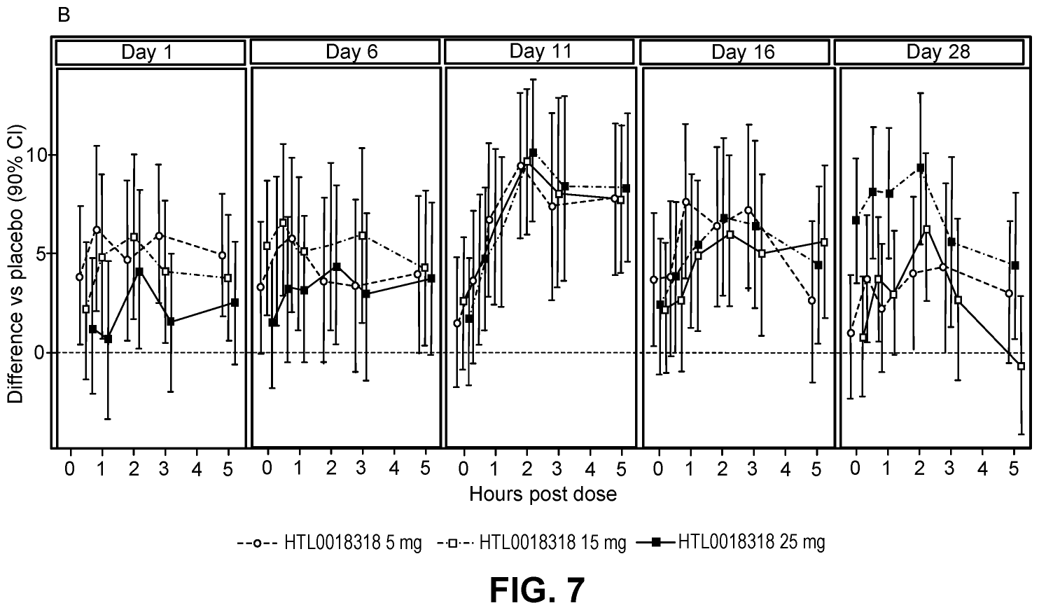

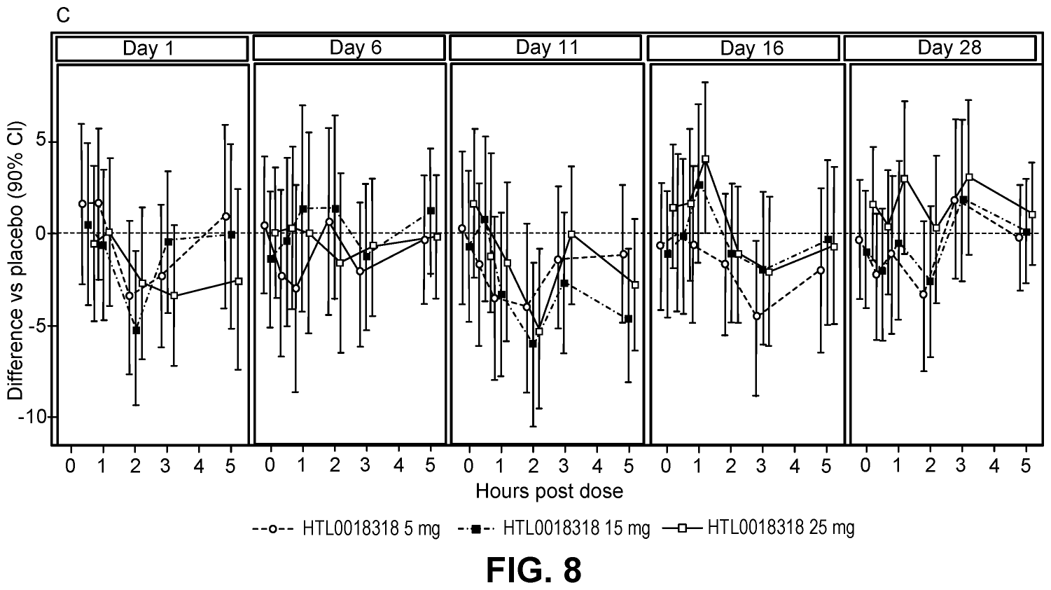

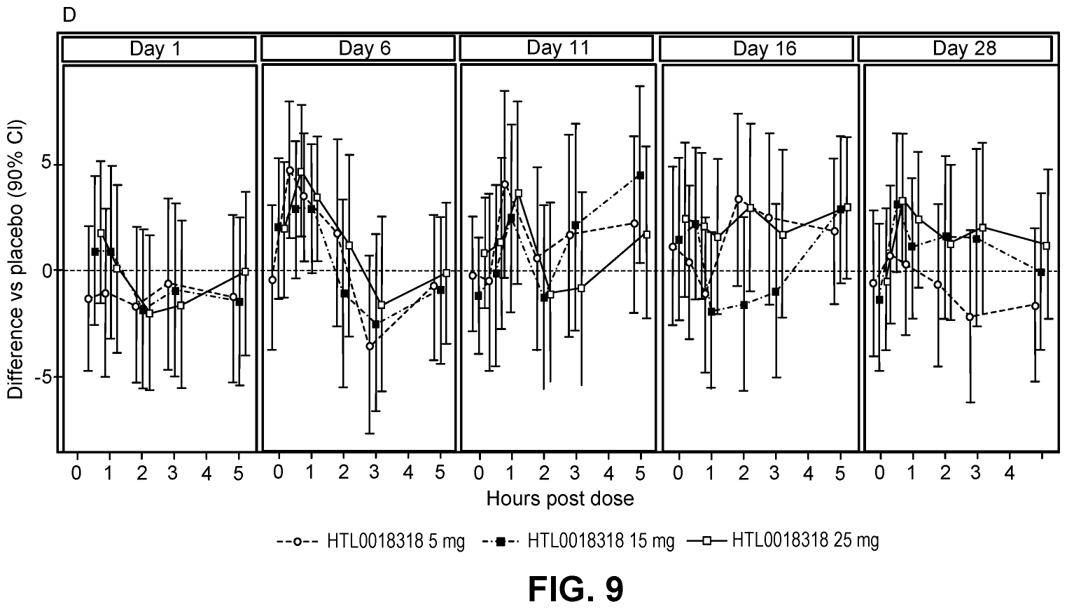

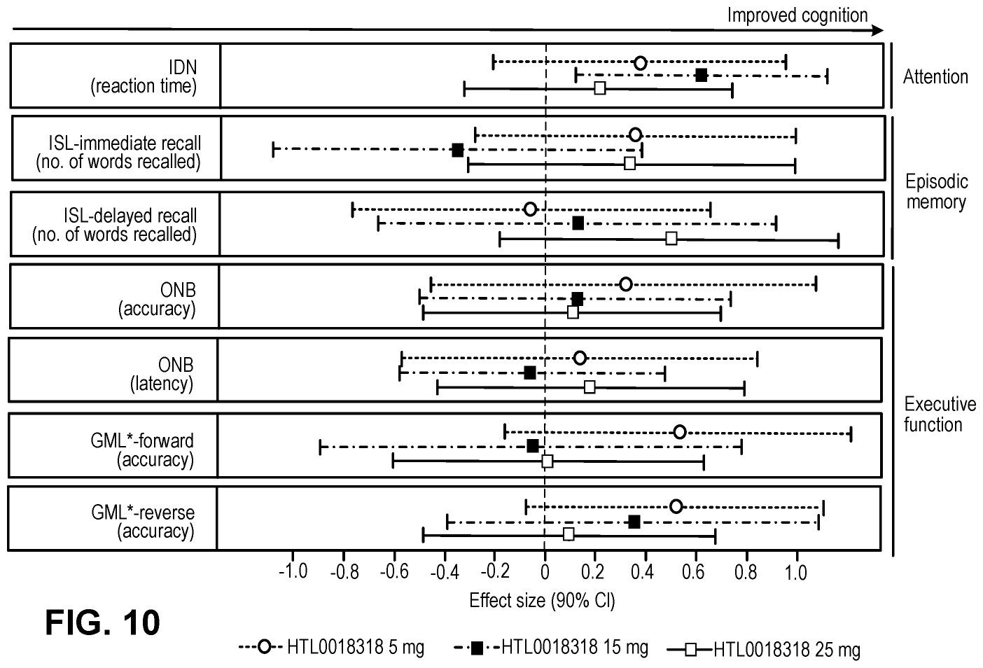

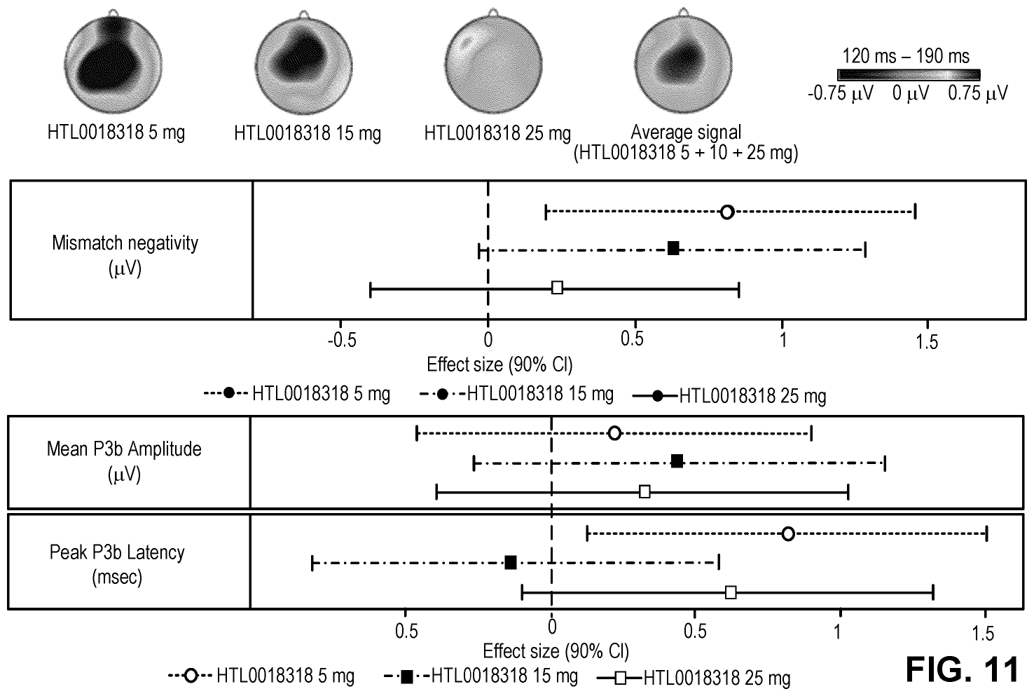

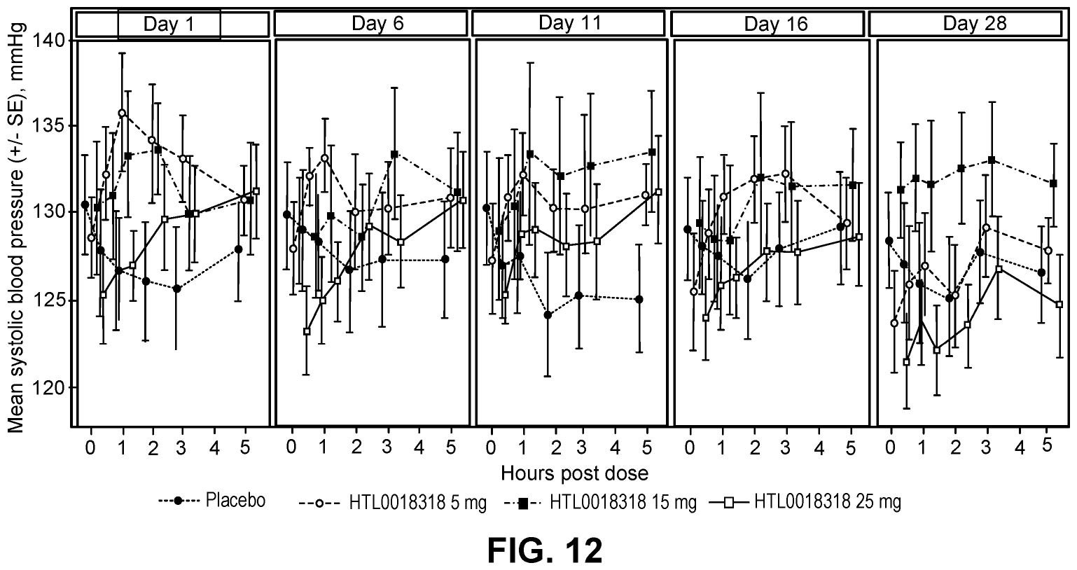

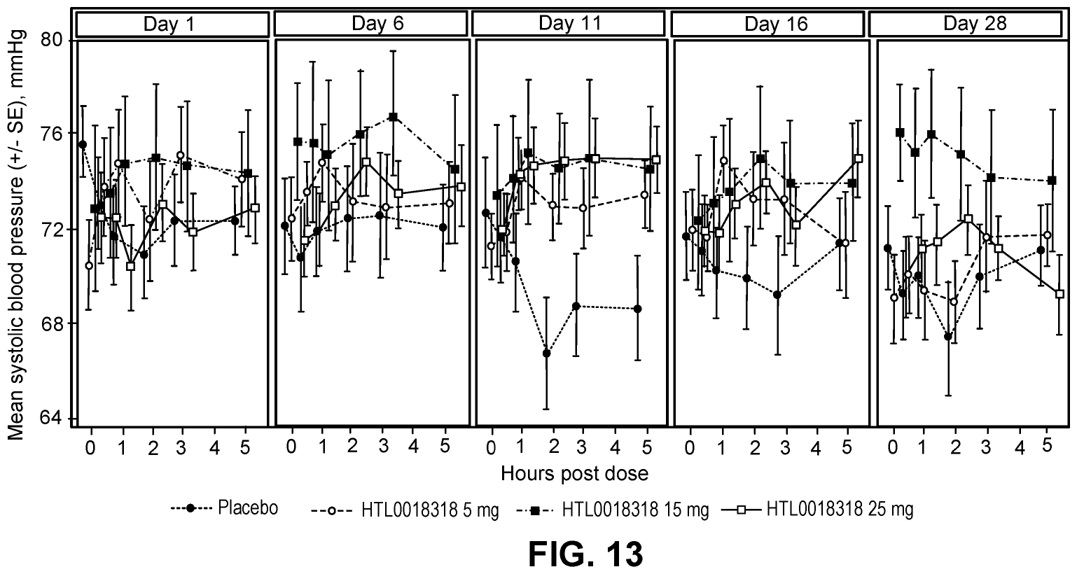

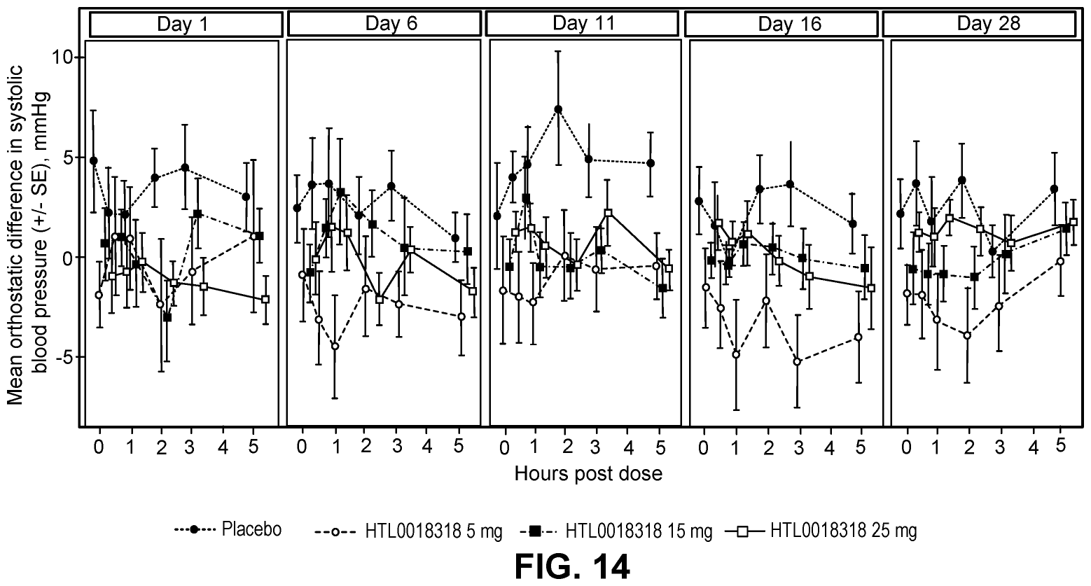

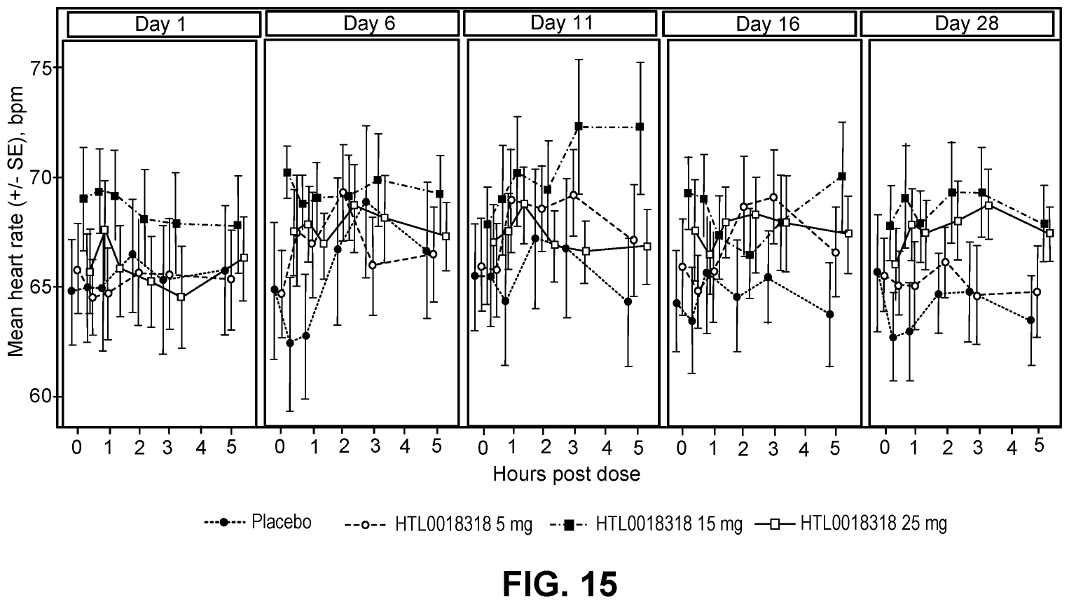

1. A pharmaceutical composition comprising the compound ethyl 3-(3-oxo-2,8-diazaspiro[4.5]dec-8-yl)-8-azabicyclo[3.2.1]octane-8-carboxylate, or a pharmaceutically acceptable salt thereof, for use in the treatment of Alzheimer's Disease or dementia; wherein the load dose of the compound or pharmaceutically acceptable salt thereof is between 5 to 25 mg.

2. The pharmaceutical composition for use according to claim 1, wherein the compound ethyl 3-(3-oxo-2,8-diazaspiro[4.5]dec-8-yl)-8-azabicyclo[3.2.1]octane-8-carboxylate is: 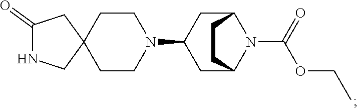
or a pharmaceutically acceptable salt thereof.

3. The pharmaceutical composition for use according to claim 1, wherein the compound ethyl 3-(3-oxo-2,8-diazaspiro[4.5]dec-8-yl)-8-azabicyclo[3.2.1]octane-8-carboxylate is: 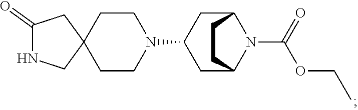
or a pharmaceutically acceptable salt thereof.

4. The pharmaceutical composition for use according to any one of claims 1 to 3, wherein the compound ethyl 3-(3-oxo-2,8-diazaspiro[4.5]dec-8-yl)-8-azabicyclo[3.2.1]octane-8-carboxylate is present as a hydrochloride salt.

5. The pharmaceutical composition for use according to any one of claims 1 to 4, wherein the load dose of the compound ethyl 3-(3-oxo-2,8-diazaspiro[4.5]dec-8-yl)-8-azabicyclo[3.2.1]octane-8-carboxylate or pharmaceutically acceptable salt thereof is 5 mg.

6. The pharmaceutical composition for use according to any one of claims 1 to 4, wherein the load dose of the compound ethyl 3-(3-oxo-2,8-diazaspiro[4.5]dec-8-yl)-8-azabicyclo[3.2.1]octane-8-carboxylate or pharmaceutically acceptable salt thereof is 15 mg.

7. The pharmaceutical composition for use according to any one of claims 1 to 4, wherein the load dose of the compound ethyl 3-(3-oxo-2,8-diazaspiro[4.5]dec-8-yl)-8-azabicyclo[3.2.1]octane-8-carboxylate or pharmaceutically acceptable salt thereof is 25 mg.

8. The composition for use according to any one of claims 1 to 7, which is co-administered with a standard of care cholinesterase inhibitor.

9. The composition for use according to claim 8, wherein the standard of care cholinesterase inhibitor is Donepezil, wherein the load dose of Donepezil is 10 mg.

10. The pharmaceutical composition for use according to any one of claims 1 to 9, further comprising one or more pharmaceutically acceptable carriers or excipients.

11. The pharmaceutical composition for use according to any one of claims 1 to 10, which is in the form of a tablet.

12. The pharmaceutical composition for use according to any one of claims 1 to 11, which is suitable for oral administration.

13. The pharmaceutical composition for use according to any one of claims 1 to 12, wherein the compound ethyl 3-(3-oxo-2,8-diazaspiro[4.5]dec-8-yl)-8-azabicyclo[3.2.1]octane-8-carboxylate, or a pharmaceutically acceptable salt thereof is administered at a daily dose of 5 to 25 mg.

14. The pharmaceutical composition for use according to claim 13, wherein the daily dose is 15 mg.

15. The pharmaceutical composition for use according to any one of claims 1 to 14, which is for use in the treatment of Alzheimer's Disease.

16. The pharmaceutical composition for use according to any one of claims 1 to 15, which is for use in the treatment of dementia.

17. The pharmaceutical composition for use according to claim 16, wherein the dementia is dementia with Lewy bodies.
